SEQUENCE LISTING (111 sequences)

SEQ ID NO: 1 - DNA, 1037 bp, Mus musculus
  misc_feature 11..11: unknown nucleotide
aaacaaaatcnggaaacctcccnaaaaaccccgcgagctttgggaaanagaccccaanngcagaaaaacccttaccgaaggcatnnacncncgcnttttncaancgcnggcgcnacaaaacggctcgagngggncnctttaagacggnagagngggggntccctacaggcangaccgggggcaagnnaanangacngnnggtagannccagcgagggtcacancatgggaacngnaaagncaanggaggagganctgccgacagaggccagaancaccgggaaacncacnccactggnggcgngcggaanggaatgcagcgcgccgggcgcntggccatganaggncagccagcgactgaagagangccnccnacnaggagggagnctgcccanacganggngggngnaancggangcgcaangcgtacaaaagcactacaacaangggcagagggaacgggncaacangnacgagcnanaccggcanagncaaaaactccggaaannngcggggccntacaacaaggcnanaangaanggngnnaaggnggaccaannnatggaaccccagggaaccnnacnacngcgggggnnggggancgccaaacggaacangggacaccngggnccgaagnanngcgnnnncgnctngnganggngaaaccagnnncaanggggcngnacncaaanaanagggccaaaanncggganaaaaagnaggggagggnnggaacncacnnaaaaacnntnnngngnncannngggccccggngaacnggganaaaagagacanaaanngggccaaacccagacnnttnananaaccnnccagggnnanaacccnnnggnggannnaaaangnaaaaaanaccanccgggggggggcananagnnacggggancgaaaaacggcccaanaanagcggganagaaaganaacacgggacaccaccannnnanaannncataaccgnnnnaaanngcncannaaacaacaccnaggaaaaaggngaganctacgcgggaaaaagccnca

SEQ ID NO: 2 - DNA, 603 bp, Mus musculus
  misc_feature 5..5: unknown nucleotide
tgtcngcggcgcatgtntggttctgagaattcgcctttcaggcggccgccgggcaggncttnggcgcccangaagcctnanaaaagaagaaggaaaaccttaaccgnggagtagtttatctggcccctaccttcacactttctgaaagccacatctataactactgtgcccagtttggcacattagcagattcagattgctagaagtaagcggcctggaaacagcaaaggctatgcctttgtggagtttgagtctgaggatgttgcaagatagttgcagaacaatggtaactccttttgggaaagacttctatcatgaaatttatccacgaaaaaaagccataaagccttttcagccagaggaatgnctgttcaccggcatcattccagagtnacncttaatcggaacgaggcattccaatgtgnagatgganataggttaaaagaagaaaatactccgaaaactactgaagggatgatatagttcctatgtctctcgcgaccctagcatcncntgcgcgtctaggtcactcgccactgtntntggnnatggactanntgngatatgctgtgtctggaatgtcgtcatccntcacgctangac

SEQ ID NO: 3 - DNA, 243 bp, Mus musculus
  misc_feature 3..3: unknown nucleotide
tantttggtcgcggnncgangtaggcgcagcatcagttgtatgccagtctgagcttcatattgtgacccagtctcatggaaacatcaaaaaatgaaaacagaaagaagtggtagaaagctcctaggcagtctactgcaattaaatatgtagatagtaatgaggagagacaaggcctagggacatgttgagttgagatatgaataaactattgngtcacagtgtacctgcccgggcggccgctc

SEQ ID NO: 4 - DNA, 764 bp, Mus musculus
  misc_feature 584..584: unknown nucleotide
ttttttggtcgcggccgaggtagagtaaattggatgtcacactccacacccgtcgtgatagtcttaaatctatcagttgaagtcttatcaagggaaggcagccatggccaccgtcttacaagaaaacataatttacacaaatggcagcactattggcctacagacctacatagttgagtcaggaggttttcttgtaaatcacagctgagtctttatcacgtgcagttagccagcgaatgctgaagtgtttgtagtcaccatgagagtccagcatgttaggaggaccataaacccaacaatcggttggggcagggagcaatttaaacagtttcttttgtgtttctactgtgttatgaaaaggtttcactagatatctgaggctggtctgagtcctcccaaacactgagtttacaggcaagtatcaccataccgtgatgggtgtttttcttttagggaactcatagaaatgcttgcaatttcaagaaatcagaagctattacaacgggaagaggaaaaccaggtaaactttttgttggtgttggttggtttttattaaaaagtattaatgcccgggcagtggtggnttcgcaccttcaattcccccttgtgggggagtggcagggggtctntgntttcaggctacnanggagttntnggcagcacacanataacccttcttgaaaaaaaaaaaanaaagtatggtggaaggcatttttttnggggaaagggngctttgananttaatggtnccaaaaaaaaaaaaa

SEQ ID NO: 5 - protein, 254 aa, Mus musculus
  misc_feature 9..9: unknown amino acid
FLVAAEVEXIGCHTPHPSXXSXIYQLKSYQGKAAMATVLQENIIYTNGSTIGLQTYIVESGGFLVNHSXVFITCSXPANAEVFVVTMRVQHVRRTINPTIGWGREQFKQFLLCFYCVMKRFHXISEAGLSPPKHXVYRQVSPYRDGCFSFRELIEMLAISRNQKLLQREEENQVNFLLVLVGFYXKVLMPGQWWXRTFNSPLWGSGRGSXXSGYXGVXGSTXITLLEKKKXKYGGRHFFXGKGALXXNGXKKKK

SEQ ID NO: 6 - DNA, 228 bp, Mus musculus
  misc_feature 3..3: unknown nucleotide
tantttggtcgcggccganggccctgaccgnttnacanngaccttctttcaagaaagnccaatngcngncttccaattttgcccatgtaatntttcaaaatggggctaaaagttaccttcctaatgcacacttggagtgccatnacaccttaactccgatatccatccacactccanagattgggttggtatattcaaggtgggatggagtcctgcccgggcgggcgg

SEQ ID NO: 7 - DNA, 218 bp, Mus musculus
  misc_feature 2..2: unknown nucleotide
tnttttngcccccggccanggacnannggtnccactgggatnaattgcctgggcangactntgaaactctctgatctnaaaacaacattgtaattagcatcatgcagaactaattgccagatttcaatatgcactctccaagtggctgggaggtgaattgcagaggtgttaaatctgggctcagatcctgtctctcatttacctgcccggcggccgnt

SEQ ID NO: 8 - DNA, 230 bp, Mus musculus
  misc_feature 3..3: unknown nucleotide
tgntnngccgcccgggcaggtacaanttgacaaaaaggctttggtcagtctaaaaaagaaaccatctnaaagatgaaagctggagggcaacnnaggaaaaacgtaanaataagttcactngtcntacagtcaggtttcccaggcccaaaggtagngtccctnngggaggatcttttggtntttgctgnccttccntgctgaaagtnatttaacctnggccgngaccnccc

SEQ ID NO: 9 - DNA, 157 bp, Mus musculus
  misc_feature 22..22: unknown nucleotide
tattttggtcccgggccgaggnaccgcgggttgtaaaacantatcttnntctctcacaaggctcgcattcactctgagtgtgcccccatcataaccattgcaaactctgaaaaatgagtcaagccctaatcaaaataacctgcccgggcggccgctc

SEQ ID NO: 10 - DNA, 374 bp, Mus musculus
  misc_feature 6..6: unknown nucleotide
tctttnggccgcccgggcaggtactttcggctggctggatagggatctttgggggaatcctcctgtctctgtctatttgactctgcaggagcacactactgtccctggcttttttttatgtgtattctggcaatagaacccaggtcttgcttatttagctagcactttactgactaagccatctcagcccctaatgtctggtttgtttatttggnngtttgtttgtttttgttatgagacaagatctctctctataatcctggctgtcctagagctctctttgtagaccaggatggcctcaaactcacggagagctgtctgccttttattcctgagtgctggaataaaggtatgaacctcggccgcgaccacgc

SEQ ID NO: 11 - DNA, 318 bp, Mus musculus
  misc_feature 6..6: unknown nucleotide
tgtttngncgcccgggcaggtacaatggtggtgtatagggcaaaangggcaactcttgagaagttggctcgtcctgtggtggaatccanacagtgaactccagttgtgaggtttgtgcagcaagcgttcttaccttgttgcaggagggttttttttttttttgggggggggtgggggttcctgcttgaggtttaataaagaggangacagggagacatntgttagcctttttgctaggggcagtctactnagccttctagactccccaagagtccctgtagcanttttgacctganntcacctnngtcgngaccacgc

SEQ ID NO: 12 - DNA, 211 bp, Mus musculus
  misc_feature 3..3: unknown nucleotide
tgnttncccgggcaggtactgtctttgtgccctctggttagtctggctaanggtttatttaatcttgattttttcaaatgacgagctcctggttttgntgattctatacccagctgctccactgttcctgtgccctgtgcattcttgggtggtcccactttacacagttattgaagcgaaacttgtcatctcacctcggccgcgaccacgc

SEQ ID NO: 13 - DNA, 710 bp, Mus musculus
  misc_feature 3..3: unknown nucleotide
tantttttcgcccgggcagggacttacagaggcactagatcaaacttnagaaggaanggaaacgnatggnatngggatttanatgtnntgtannataatggtacaaaancacncttacatgnttncacatacaggaattaaaaaggggaattaaagtcggaaggcnaaangggagctattaaaganggaganggtgaaacanggntgnaggatggngatnaacactaatgggggactgttcttaaaagtatattatatntacagatntcttataataaaacccaatcctctacagagtattatcaacataanctntaaacngttatctgngagggtcanaggcaatccatntaagggaccttgcttgcaaaagatattttaaaaagttgttttatcttatgagaaagaaaacatgcttttgaaacagntggtaatagccaaaaaaacgtttaaaggctgaanactgctgaggaactggatgacaaactgcantnggtgtcagttaaaagcgctccgtcanggtgggtgggaccagacttaatgtagctacgggactactcttgggcagnagctggcnggacctgtaganaaaagnncangcangtgcagcttggggctaaatcctgnggnnttaaactccanggttgcnaggntgntaagcntngggggtaanatnattcnnnngaacctaggataacntgggngtttgg

SEQ ID NO: 14 - DNA, 282 bp, Mus musculus
  misc_feature 24..24: unknown nucleotide
tttttcggcccgccccgggcaggnacttnnaaaggcgnnnatttttttcaacttactttgntacactancncttatctatgtctaagcaacagncctatatntggctntntgaacactcagtctaaaccatgagagtagaggagtggcatacagtcagactgatcaccaggtttatttagtttggggtagtagactatgtggtaaaccacatcagaagaatggcagatcttnaggnctgtcancagaggccaatagccttctcacctnggccgcgaccacgc

SEQ ID NO: 15 - DNA, 232 bp, Mus musculus
  misc_feature 145..145: unknown nucleotide
tgttttggcgcccgggcaggtactttttttttttttttttttttttgagactgggtttctcttttagccctggctgtcctggacaaggcaaatcttataaggacaacatttaattggggctggcttacaggtccagaggttcagnccattatcaaggtagaagcaaggcaatatctttttatttaaagatctatttatttattatatgtaagtacctcggccgcgaccacnc

SEQ ID NO: 16 - DNA, 654 bp, Mus musculus
  misc_feature 2..2: unknown nucleotide
tnttttggtcgcggnccgaggtacactagtactctattgncccgcnccaaaagngggatgtcatnaaccgacatngnggccaagaagagnaatgagggagggcagtagcctattcacgggagctgactaggccacaaagggaggaggtnnntgccttagatgcaggcntggncttntngatacagtaactacgggatntgngggttattncnatatntatcnnaatgagggaacncccagntntgagacttcanannncactttaccncntaggaaacacatancanttcacctaantacancactntgacgannctggaaaagcccacaccagaataaccctgatnccacataanncnnagancnnnaggncggnaggnggnaggnanccttgangcaaacccatgtgccatctgtgattgccccttcagtgactagaannaaagcccagaaaagtgcanacaacacaagggcnggaatacataggagaanacagttatancaaaagtaacccgataatnggntggnganatntagncccatctttncttccnggggcttngnacaatncnnagggncccaantgggggagaaaaanggtgacnccaaccantgaaagncnatcnanggttccacccggacccacccggnggc

SEQ ID NO: 17 - DNA, 466 bp, Mus musculus
  misc_feature 2..2: unknown nucleotide
anggtaaaaaattttttggnaaaaaaaagcccnttnttggtnaaaaaagacncccccccttcgnanaacgtgtttcgcgccaaaanaggtgggaaaancgggataaatacctgggcgaanaaaaaaaacgggcgcccctttttgaagaacggggggccccnccccngcgggcaaaggggaaacnnttttttttttnnncanggggggggggngggnngaaaaaaaaaaaatattgncacgnnaaaaaagggggntttacctaagnacnnggggcccacgggaagggggcgnngnaacccccggncccttcncnggggnaangttnnaacttnnnggacctttntnaagnngaaaagcnttncaaaagtttgganttaaaaggggtnaangggaaattnggctggccctttcaagaagggnaaagaacccgggccggaacatnncactaccncggccggaaccacgc

SEQ ID NO: 18 - DNA, 844 bp, Mus musculus
  misc_feature 6..6: unknown nucleotide
tttcantgcgttttatatgccngctcgagcttcgcaatgtganggatntntnnagaattcgccctttcgggggcccngcccggggggnttttntaccancctgagagccngagnaaaaccttaaaaactaaaacttttttaccaacattnatcccaggnggatgactggatgncacgtgaaanggaaagagcaagtaagcttncagagagaaacagcagatacctttatgacctttgaatgaagcaaaaattacttaatcaagggcacaaacccacaaacttaaaggaatgacattacaattacagtcacataggagctgatatctccagcgactgagcccacagcttacctgtggctgcagcattgcacaatcctcagtaagtaaagtggatggctttcaggcgctgatgaccggtttggcggcaggccacacccaccaaccattcccagatcttgcccaatgcgagggtgagggtaccttgcccgggcgggccggttcggggcaggtaccgccgggcaccaccaccntgtaacaacaaccaaaaaccaagaacttaaaggaagatagctttaanccaanttnaananccacttaaccttggttctttggcaaaaanggacccggtccctggaggttttggaccttccccaataattccaattttaaggggggctttaacaaaaaaaaattttaaatancccctantttttgnaaccttggggccgnggaancccccccttaangggngnaaatttccaggncaacttgggggggccggttcttaaagggaatcccggctcgggganccaaacttggaggcataantgggggttttttanggg

SEQ ID NO: 19 - protein, 281 aa, Mus musculus
  misc_feature 2..2: unknown amino acid
PXKTPXYASKFGSPSRDSLXEPAPPSXPGNXXPLRGGFXGPKVXKXRGYLKFFFVKAPLKIGIIGEGPKPPGTGSXFAKEPRLSGXXXWXKAIFLXVLGFWLLLXGGGARRYLPRTGPPGQGTLTLALGKIWEWLVGVACRQTGHQRLKAIHFTYXGLCNAAATGKLWAQSLEISAPMXLXLXCHSFKFVGLCPXLSNFCFIQRSXRYLLFLSXSLLALSXSRXIQSSXWDXCWXKSFSFXGFXXALRXVXXPPGXGPRKGEFXXXPSHCEARAGIXNAXK

SEQ ID NO: 20 - DNA, 338 bp, Mus musculus
  misc_feature 3..3: unknown nucleotide
ganattaatngttgaatccgtcagcttcggaatggaaggnatctgncaantccccttagccggccncggccaggcnttttntggcctgaaaatcntggggancngctgacctggaaccctcactgggaaggatgcagngcaaaaaaaagctgngcanaattcctgcggntgctacagtgngtccagcgtcctgctggctgtgctgangctggacagtggcgcatcattcaagtgcacagttacccatcctgagtctgacaccttaactggcacaattgcaaaatcacagngaacaccttccaccccaggtccacctgctacctcggccgngaccacgc

SEQ ID NO: 21 - protein, 112 aa, Mus musculus
  misc_feature 1..1: unknown amino acid
XLXVESVSFGMEGIXQXPLAGXGQAFXGLKIXGXXXPGTLTGKDAXQKKAXXNSCGCYSXSSVLLAVLXLDSGASFKCTVTHPESDTLTGTIAKSQXTPSTPGPPATSAXTT

SEQ ID NO: 22 - DNA, 279 bp, Mus musculus
  misc_feature 7..7: unknown nucleotide
taaccgnggnccccggcccaaggnaccttttttgcancaaaaatgctgggtcctgtttggcctanggctttggttgnttggttggttgtttttccanacagggtttctnttgtatagacctggctgctctggaactcactctgtagaccaggctggcctcgaactcanaaatccacctgcctctgccttccaagtgctgggattaaaggcatgcaccacaaccacccggntcagtctatgtctttttattgcccgcgtacctgcccgggcggccgntcg

SEQ ID NO: 23 - protein, 53 aa, Mus musculus
  misc_feature 8..8: unknown amino acid
NRGPRPKXPFLXQKCWVLFGLXLWLXGWLFFXTGFLLYRPGCSGTHSVDQAGL

SEQ ID NO: 24 - DNA, 441 bp, Mus musculus
  misc_feature 18..18: unknown nucleotide
aagcggggccccggcccnagggacttttttccctgccccccttaaanngnttttgaaaggggggnngnaaacataantacggncccggnnatngngtcanannganaacannngcggtggagaangaganntcngacctnnagaggggnnncnncccacaggtngagngcaanacctctagantntcccanntgaccacaagaaannctcagngttngcnantanaactgtgagagcggngcgngtatgtgcaactcanggtgggggatcacccctgataatcctggacactgtgggaggcatnatgcagggcnggactttgngacgttctnggccatcctggnctataaagngagcnccnagacagngcanngntanncatncgaaccccngaccaaantngatncanagantggantntnnccntgggcccnaccacgc

SEQ ID NO: 25 - DNA, 903 bp, Mus musculus
  misc_feature 2..2: unknown nucleotide
gncganattcataggccgtagatctgtcagcggcgcatggatggatattgaaaattcccctttgagcggccgcccggccgggcccggatagacctttggaaaagggatgggaagggtggaaggctttttnaantggcatggggaagttccacaggcctctggcctggttcttgggggggttctggctagacatcattaaagctcttggccaaaagttcaatatttccctccttagagacagaggttggaaatgccctgcctgtaagtgttgctaggctgggtctcaacctcctggggctgaagctctcctcttacctcggccgcgaccacgctaaggggcgaattccagcacactggcggccgttactagtggatccgagctcggtaccaagcttgatgcatagcttgagtattctatagtggtcacctaaatagcttggcgtaatcatggtcatagctgtttcctgtgtgaaattgttatccgctcacaattccacacaacatacgagccggaagcataaagtgtaaaagcctggggtgcctaatgaagtgagctaactcacattaaatggcgttgcgctcactggcccgctttccaatccgggaaaaacctgtcgtgccaactgcattaatgaatcgggccaaccccccgggagaaggcgggttgggttttgggcgcttttttccctttcttggttacttgactcgttggctcgggcgttcgggttcggggagccggataagttacttaaaggcggggaataccgggtttccccaaatccgggggttaccgccggaaaaaaaattttnnnaaaaggggccnaanggnnggnannccccaaaaagggccgggngggggntttttanannnccccccccnnnnnanaaaaaaaaancnnngnn

SEQ ID NO: 26 - protein, 300 aa, Mus musculus
  misc_feature 2..2: unknown amino acid
RXSXAVDLSAAHGWILKIPLXAAARPGPDRPLEKGWEGWKAFXXGMGKFHRPLAWFLGGFWLDIIKALGQKFNISLLRDRGWKCPACKCCXAGSQPPGAEALLLPRPRPRXGANSSTLAAVTSGSELGTKLDAXLEYSIVVTXIAWRNHGHSCFLCEIVIRSQFHTTYEPEAXSVKAWGAXXSELTHIKWRCAHWPAFQSGKNLSCQLHXXIGPTPREKAGWVLGAFFPFLVTXLVGSGVRVRGAGXVTXRRGIPGFPKSGGYRRKKNFXKRGXXXGXPQKGPGGGFLXXPPXXXKKXXX

SEQ ID NO: 27 - DNA, 462 bp, Mus musculus
  misc_feature 2..3: unknown nucleotide
tnnaccggcccncccgggcagganctttttttnnnnnnnnnnnnnnnnnnttnnncccaaagcaccnggaagactnccccttnctccagancntcaatttttcctcccagggggtcccncnaagtcananatntttttaagggancctgagttccttgctncaaatttcggnttgncanttaaaatggatgaaaacaggtntggaancnggaaaaccnttaggggggggttgccgaaananatcgtaaaggcttaaactaaaaaaaaggaaatggggttggaanaggtccaaggatttaaaaatgcntttagggcattgtnttggagtntgggggttgattnttgaataagggggctnaaagagggggaggagtcaaggatgggcaggttttgggtctgggcaaacgggtgacttggggtgcaatttaccaanaaggcccgngtccttggccgggaccacnc

SEQ ID NO: 28 - protein, 153 aa, Mus musculus
  misc_feature 1..1: unknown amino acid
XPAXPGRXFFXXXXXXXXPKAPGRLPLXPXXQFFLPGGPXKSXXFLRXPEFLAXNFGLXXKMDENRXGXXKTXRGGLPKXIVKAXTKKKEMGLEXVQGFKNAFRALXWSXGVDXXIRGLKEGEESRMGRFWVWANGXLGVQFTXKARVLGRDH

SEQ ID NO: 29 - DNA, 452 bp, Mus musculus
  misc_feature 38..38: unknown nucleotide
tcaagcgggccgcccggccagggaccttttgctggggngcagcctntttcagctncttnatgggntctgaatcttganataggccatcgctctgttagctggnagnagggcgttggngcggtccgctgctatcccgcgggtgcagcactcaatggcttgctcgtatttcccctcttcgaaaaatccgtngcccaggtctttntccgctatggccttctgcctgccctgctgaccaccggttggtttgctntcccctgcagctggcttactctccgcagcagcagctgctccaggacccgagttttccttggacgtcaaagcctgattaattttcctgagttcattggttgcttcaaagttatctggctccagttctaacactttctcataatcttttctggcgcctctaacttctgcaagcaaaccgagctgcacctcggncgggaccacgc

SEQ ID NO: 30 - protein, 150 aa, Mus musculus
  misc_feature 4..4: unknown amino acid
VVPXEVQLGLLAEVRGARKDYEKVLELEPDNFEATNELRKINQALTSKENSGPGAAAAAESKPAAGXSKPTGGQQGRQKAIAXKDLGXGFFEEGKYEQAIECCTRGIAADRXNALLPANRAMAYXKIQXPXXSXXRLXPSKRSLAGRPAX

SEQ ID NO: 31 - DNA, 469 bp, Mus musculus
  misc_feature 2..2: unknown nucleotide
tntaatgccgctcagctncgcctgtgaaggaatntgngaantcgcctttnggcngcccggccgggaaccctttttttttcctnngccnanntnnaannaccaangngnaaanntaaacccaagagntttgatcacctagcnggcggccataanatccagtaangccaagggggttntaaaccttccaaggctgggatccttaaaaacaagntcctantgcagnggtgacccccaccatagnannatggaattgctactttgnaacnatnnaannngaaatacaaagtattctgatatgcaggggnatctaatatgcgacccccncaaaggggnnnnnncnnnnnnatnnnnaaccanggnggnaagctaaaatgcccatactaagatccagagagtctcactttggngggccctntaaatcactctgcatctaantctgcattcaaaatcacctcggccgngaccacgc

SEQ ID NO: 32 - DNA, 384 bp, Mus musculus
  misc_feature 19..19: unknown nucleotide
ttgtctgctcagcggcgcntgtgnggnntttgcaatttcccnttcntggcccngccngggaatttttnttgntgaattcccngncggnnaaaaggggcaattggccaatttgttccccgggggaaacanccacttntaaaaangngaaggaaaaggccnggaaaantnggntgacctnaangggctggaaaacnnggaacngggaagttnggagctaagtgctgtaaaaccccagcccgtntttgaagcctcagctgccccanaaatatacagcgccnataaagatcccgctccagaaacacccggggcggaagacaaatgcaggagttctaaggccaagcccttccggtgtaagccttgcagtacctgcccgggcggccgntc

SEQ ID NO: 33 - protein, 127 aa, Mus musculus
  misc_feature 8..9: unknown amino acid
CLLSGACXXFAISXSWPXXGIFXXEFPXGXKGQLANLFPGGNXHXXKXEGKGXEXXXDLXGLENXEXGSXELSAVKPQPVFEASAAPXIYSAXKDPAPETPGAEDKCRSSKAKPFRCKPCSTCPGGR

SEQ ID NO: 34 - DNA, 769 bp, Mus musculus
  misc_feature 8..8: unknown nucleotide
aatttcantgcgcncttagatgctgctcgagcgggcggcaatgnganngntttnngnnnaaatccncccttacnggggncgcggccgaggggntttttccagggntggggtngggcatgtaanaagcatnggaagtctcaagtagcactctnggggacttttctgagctngctctcctcaagggttgggctaaagggcaatttcttaaggggaagctgctngaggtataactgtnaataagttngttttcctgctggggactaaaagaaccagctctggacaacaactaacaaaatgngctaacatgttncccctanaaacagagctctgngcccttcaaagttcctctggtagggatgcactttctcttttcttcggggcatggtatgctttgcacatatcatgagccgttcgaaagcagagaagggtgtgccaggccctgtacctgcccgggcggccgcccgggcaggtaattggaaaacacatcggtagaagcatcttctctagtagccttaactggctctattaatagccaacatataattatgagaaggcaagtnccaaaaaatatccagcaatagggaagaatggttttttttcccaaaaagaacatttttctggaattaaaagcaaatgggcaacactacaaaaatggatttactggggggnaaaatttaaanggacctcgggncgggaacccccttanggcgaaatncaacacactnggnggggccggactaaatggggancccactcggnccccaactgg

SEQ ID NO: 35 - DNA, 442 bp, Mus musculus
  misc_feature 14..14: unknown nucleotide
gcgttttagatgcngctcgagcggtcgcantgtgannggtatntgnnnaantcccccttagcggggncgcggccgagggacttttttttttnnctttnnnaannnnnncgcccacccggngntggnttggcttcnagncccctaaaatagatccacacttntgtcctggaatatgcttctnaaattagnaaatgtcccagcactcananngtaaangttcttaaaaccgngggcaaaaatatctatgcaaaacgcacccantagggaatgtgagtgtgtgcctgtgcttgtgtggctccagacatctgngctcanaacttaaccattaattnccaagccgacctgttggcccggcttctgagcttggtgaaagttggtgactgagagcgctttgtggagtagaaaccacagacaatggtttgtacctgcccgggcggccgct

SEQ ID NO: 36 - DNA, 703 bp, Mus musculus
  misc_feature 8..8: unknown nucleotide
ttcgtggncccgggccagggacctttttttttnnnnnnnnnnnnnnnnnnccaattnaaccgntnaatttaaaaancttaaaacnggnttcccgngaaacanttttnanggggcaaaatttttcataaanaataaaataaaccttactanattttgcaaatnctgaggggggcnggggaaagctnttttggncaacaaagtttttcccacagntgggncagctgggaacaaaangctaactancgttttttnttaanagaacntcnccncccanctgganagctggggactagcnctctggtcaacctgcccgggcggccgcccgggcaggtnccacccngactntagtggcngtggcngctnactgaaacccctggnttccacgcttanaacgaattgcagcccntattctgtctgaaatntagaggctccagagagatgctgagtccactgngctggacccagactggttgctgggttactttttggggtttnttttattaaaaatttctatcctttttctntttnganggcacaatcttatgattcccatgcctgccaagaggnatcttccaggtggggccaccncacccttaggttggagtcaaganacttcttccttttaatccangggcccctganngggccggcacnttgggctggnnggntcctttccccccttcttttaaaatngggccncggg

SEQ ID NO: 37 - DNA, 1091 bp, Mus musculus
gtagattttgaacctttactatagggcattatgggccctctagatgcatgctcgagcggccgccagtgtgatggatatctgcagaattcccccttattttggtcgcgggccgaggtactgccgttctgcaaatattcagttcagaactattcatcaccatgaaccaaaagtaaaagaggaaaaaaaagactttgaagactcaatggacgaagctctaaaagaggccccggaaatgcctttgctggacgtgaagagtgagcccgaggagaatacggactcaaacagtgaaagcgacagagaagacacagagctaaagtcgccaagagtgagttcttgtagctttatctgcttcaagcattagtttgaactttttgtgggcattttaatgtatgtgcatactatttgtttatgttccagtatttagtaatgttgcttcaaggttattagagcagtctctttctacagacagtacttatcagtcctacctgcccgggcggccgccgggcaggtacgcgggggtgggacagaatgtgtttcagtgcctttgctcctaggaacaagcgtaggtgccaggataggtagaaagccttccttggttattatacagcttaattccatctatggagagtctggaagtttgatggtttcctttcttaattcccttaatttttttaccactcaatttattcatgccccatattgtattggaagtcaaaagggaaatagtgggggaatggttctcttttcccaaatgcaggccttaaggattgaactcaagacatctggcttggctatagatgcctttccctgctgatccatctcaatcctcctggcataattttaacactctctgttgaaggagaggtctttgtatgtggctctgttcattttccaggggctctctgtctccttccttagatactgttcagccatgttgctgttgacggcaggactgggccagttactgaagagttccctccaccacacaaaggtcacctcggccgcgaccaaactaagggcgaattctgcagatatccatcacactgcggccgctcgagcagcataagagggcccaatcggcaagtaaggtc

SEQ ID NO: 38 - protein, 305 aa, Mus musculus
  misc_feature 88..88: unknown amino acid
FGRGPRYCRSANIQFRTIHHHEPKVKEEKKDFEDSMDEALKEAPEMPLLDVKSEPEENTDSNSESDREDTELKSPRVSSCSFICFKHXFELFVGILMYVHTICLCSSIXXCCFKVIRAVSFYRQYLSVLPARAAAGQVRGGGTECVSVPLLLGTSVGARIGRKPSLVIIQLNSIYGESGSLMVSFLNSLNFFTTQFIHAPYCIGSQKGNSGGMVLFSQMQALRIELKTSGLAIDAFPCXSISILLAXFXHSLLKERSLYVALFIFQGLSVSFLRYCSAMLLLTAGLGQLLKSSLHHTKVTSAATK

SEQ ID NO: 39 - DNA, 750 bp, Mus musculus
  misc_feature 3..3: unknown nucleotide
gtngatttgacctttacttagggcttcatgggccctctagatgcatgctcgagcggcccgccagtgtgatggatatctgcagaattcgcccttgtattgccgctaccgggccaggcccggactttttttttttttttttttttttttgggaaacgggggacagtgtctccgagaaagacgttttgcttccactcaaaatattctttctttcacaggtttttgcagaaaaaaagtcaaggagccggctcgggaatttctacgtttcttctgagagcatcaaaaaaaggtaagccagggtgtgtccttgacttcctacacacaagcggaacttccgggaagattcccgtaacaagggcggccaagccgtgacagcaacatgtggcatttatggagagagatccgagccgtagctcaggggcaatgcttcagagtcccttactgaaggcaaaggaaagtgggggagaggcaggagcaccccagattattgtacctgcccgggcggccgctaagggcgaattccancacactggcggccgttctagtggatcccagctcggtaccaagcttgatgcatagcttgagtattctatagtgncacctaaatagcttggcgtaatcatggcatagctgttnctgngngaaattggtatnccgtcacaattcccccaacatacgaaccggaagcataaagngtaaagcccgggggcctaatgagggacctactancattattgggtnggctcactgccg

SEQ ID NO: 40 - DNA, 324 bp, Mus musculus
  misc_feature 6..6: unknown nucleotide
taacgngggccccggccnagganntttttaggggncngtttttntatggnntataaaaggggttatcattacattnatactggctgaaaatgagtagctgaactactcgttctctgttcgcctccattactgctctaattngntaaagccttacgtctcaaaatgggaagacaattagcctttcattatttgttacattcaactgaaggcttttaaaactatgactttaaaatgtagctgktttgngccctcagtctttgnaaactttgggattcattggtcatgtgaatttttcatttgtatgtacctgcccgggcggccgct

SEQ ID NO: 41 - DNA, 680 bp, Mus musculus
  misc_feature 101..101: unknown nucleotide
attcattgggcccttagatgctgctcgagccggcccctgtgaaggatatctgcaaaaattccccttcgtgggcccggggccacgggcctttttttttttcnggttgngggggscaattcattggggrrawtctkttktkgkcwggtttmarrcmtatgkyttctttttacttcwmwgaaagsrcacakgyywtttwggkkytggggctaaggrcargrtsyttancaagaamytkawgracacyywttggccmmtgaatgacatgttgwaatmmsgktkgwtcsgtgttyggrtcggccyttggcmggayyssmtccccataggccctgtggcgngcttttgngtttaaacagctnaaaangctttggccgacaancaacccngggtcctgcccggnggcgttaagggaanttcacnactggnggcgtctaggggaccgactcggaccaacttgtcatacttgngtttnttagggccctaaatacttggngaacatggcatantgtcctgnggnaattgtatcgtccaatncccaacatccaccggaacatnaaggggaaccnggggctatgaggactactaataatgggggntcctgccgttcagnggaaactgngccatgntatnatcgcacccgggaggggtnggttggccttccttctctactatntgctgggntg

SEQ ID NO: 42 - DNA, 292 bp, Mus musculus
  misc_feature 3..3: unknown nucleotide
gtnattttatagcgttttagatgccgctcgagcgttcgcaatgtgaaggatatctgcacaaattcgcccttaacgtgggcccgggcccggggtcctttttttaatttgggggccaactggaaacaccaccccccccccccaaaactggttcccttagggataagsgcanggccattttcnggaawattctttnaacaaganatctgactggcaggaatgcttcatttkgttcaaaggccatttacccccaaaccaccccacctggngcattgnacctgcccgggcggccggt

SEQ ID NO: 43 - DNA, 524 bp, Mus musculus
  misc_feature 6..6: unknown nucleotide
gagaangtagaattttcgaaaagcctctgctaaatgcccgctcgagcggctcgccaatgtgatgcgatatctgcagaattcgcccttaacgtggggccgcggcccaaggaacccttttggtgcaaacgttccaacctgttaatncgtaagtggngaaaaanctgnaancaagttgggttcgggtgantgtgtgttgggtgtgggttngggtagggaggcanacagctccgtcgcanactacggacctctccggggtctcagcccgccaaaaccctgtccggtaggaaacactaccgggcatcatgaagtggctgtcnagtgttcgttgggagactaaatccagcagcgagggagaaagatgtggaaagattcttcaagggttacggacggatccgagatattgacttgaaaagaggnttggttttgnggaatttgaggacccaagggatgcagatgatgctgttatgaacttgatgggaaggaactttgcagtnacagggtgacnatngaacctgcccgggcgg

SEQ ID NO: 44 - protein, 174 aa, Mus musculus
  misc_feature 2..2: unknown amino acid
EXVEFSKSLCXMPARAARQCDAISAEFALNVGPRPKEPFWCKRSNLLXRKWXKXXXQVGFGXXCVGCGXGXGGXQLRRXLRTSPGSQPAKTLSGRKHYRASXSGCXVFVGRLNPAAREKDVERFFKGYGRIRDIDLKRGLVLXNLRTQGMQMMLLXTXWEGTLQXQGDXXTCPG

SEQ ID NO: 45 - DNA, 926 bp, Mus musculus
  misc_feature 4..4: unknown nucleotide
tttngcgggcggccnggcnngnncttcaaggntngcnananaacntnntnggcaccatnggcntggntngctngnaagtnatngnaggaccnagaatngnagggnnagggnaggnantttaannntccttttancaccaccncnantttggaggntaaagtcannattctggggncatcntacctttnantcaaattggnccttaagtggcaccacttgaggaaaggctggtgaatgaaactaagcccatggtcccagcaagtaagtggacttttcaagccaaggacccgggcaccaaactttcaaggcnggggttcggcaaacaagaaaggtngggaaaaggaattttttggaagcctttnccaagggaagaataacttcttggtanttaacccggaattttaacccttcgggcccgcggaccccaccgcttaaaggggccgnaaatttcccaagccaccactgggcnggcccggtttacttaagtgggatcccgaagcntcggtacccaaagcntggatgcataagcttngaggtanttcntaataagtggtcaccctaaaatagcnttgggcggtnaatcaatnggncaataagcttggtttcctggggngnaaaatnggtnatnccggttcacaaatttcccccacaaacattaccaagcccggnaagccntnaaaggggtaaaagcccngggggggccctaatgggggggagcttacctnacanttaaatgggggtngggctttaattggcccgtttttcaaannggggaaaaccctttngggcccannggnattnaaanaaangggccaaacccccnggnnaagggggttttnntntttggggccntttcccnttttnngnaaaaaaaannngggnccnggggnnnngnnngggggggggggttttttttttaanggggg

SEQ ID NO: 46 - DNA, 564 bp, Mus musculus
  misc_feature 4..4: unknown nucleotide
tttntccgantcantatanggcnagnangtggtaancancnnnnatancncgggaaatgntgaaaaactggngtccaaccaaacattactggtatttatcaacccgtttggaggaaaaggacaaggcaagcggatatatgaaagaaaagtgggcaccactgttcaccttagcctccatcaccactgacatcatcgttactggacatgctaatcaggccaaggagactctgtatgagattaacatagacaaatacgacggcatcgtctgtgtcggcggagatggtatgttcagcgaggtgctgcacggtctgattgggaggacgcagaggagcgccgggtcgaccanaccaccccgggctgngctggtcccagtagctcggattggatattccganggtcacggntggtngtatcacgngcacangccaaaactggnttattngtgtgggctctgctgtgnctancccaanaatctgttcgcgngtcgttnggantananaaacgnggtnnaaaatantaacnttcccntnangagttncaacggtaaaanccngttncaanaca

SEQ ID NO: 47 - DNA, 199 bp, Mus musculus
  misc_feature 32..32: unknown nucleotide
tttttggtcgcggccgaggtacacacatgcgngggnatgcnnatanacagggnaatgcccntcaaccaaaacaaatgaaggnaaaantgggtttaaaaagaaatgggaaggtgaaacgggcancataccgtaatcacaataagtctcaatatatgaaggggccaagaaaatggtcaaaccctgcccggggcgggcccgc

SEQ ID NO: 48 - DNA, 868 bp, Mus musculus
  misc_feature 8..8: unknown nucleotide
tttttggncgcggcccgagggactnttntttgggggggntgttcttaaaaaganacaggccccccctgggtaaccctgggatgggncttgaaccnaaaaatctgcctgccccngccnccnagggctgggaataaaaggcgnggcacccaatcccggctncatacctcggcnttggcctttctctngctcacnacttcagnccatgcccaaccctaaaagacaccngcccgggcngacgctcgaaaggggcgaatncccagcacactgggcngggcgtanctagngggaaaccgaagctcgggaccaaagcttggatgcataggcntngagtaattctaanagggggcnacctaaaataagcttgggcgggaataaatggggcnanagcctggtnnccctggggnngaaaaantggggtaangncnggttcaacaaantgcccaccacaaacaataaccgaagcccgggaaaagccaataaaaagtggaaaaaagccctgggggggggggcccnnaaaaggaangnggaagcctaaaccttcacaaatgaaatntgggngagngggcggnntaaanngggcccgggatttcccnggtccgggnaaaacccggggangggccnnncncggcaantaanagngaatcgggcncaccgccnccgggggaanaagccggcntgngnngnatggnggncnntncccgttnccgnaaancggaaatacnangnctcngactgntnngtnggcgnnanagngatcnntcacncaanagnggnagtncggcttccncnaancggggaacncccgaaaaaccttntnaaangccnaaagncaaaccaaaaggcnggcggnntntcatangccgcccnc

SEQ ID NO: 49 - DNA, 763 bp, Mus musculus
  misc_feature 3..4: unknown nucleotide
ttnngggnncgggccanggtnaaaacnnggtnaggaaggntnntnaggtnggataaantgaacaancccaggttnaaanaaaaaaatngnaggngaacagggaanaangtgngtttgaacagatcgnngatggcttgnacattggaagtagggaaagttgcntggngactggganggcntgnncactgacantggggaccacggaagtgctnataagaccanggaagctttgncttgganttaccttgcccanatnngacggcntggcagtgtnnccggcgcttganccnnantgcatgagngaccntagtccanaactccccngaaccannnggaaaggccaggngaagancancnaaanaccgcccccccagctnggcngacccccagattgganaccggaaaagacccattaccccngcttcaacggactgcgttaaatactncaacctgggcaccacaaacgnagaagcctaanccntnccttnggccgnaccccttaagggnaattcaannnactggggggccgtanttagggatccnnnttngganccaanntnnttcttgnttngggtttttattgggccnccttaanggcnnggggnaaaaannggnannnggntttccccggnaaaaatttnnnngntaaaaantcnnaaaaaaaaaaannnnaaaannaaangnantnnnggggnnaaaaangggggnnntcantgggggntnncnnnngnngtttnnnatgaaaacnnnnccccnntntann

SEQ ID NO: 50 - DNA, 394 bp, Mus musculus
  misc_feature 4..4: unknown nucleotide
tagnttggtcgcggnccgaggtactccaggaggngcagctaaaaatgcaaaatggccanaatgtgcgccnatcgcttncagttctacaacagccagactcagatctgtgtgggaaacccgagagaaaggaagtctgccttcaggggtgattctggtggccccctggtatgtagcaatgtggcccaaggcatcgtcttctatggaagcaacgatggtaacccttcagctgtattcaccaaaatccagagcttcatgccctggatcaaaagaacaatgagacgccttgcaccaagatatcagagacctgctaactccctgtctcaggcacagacctagggacttccatagcccttattaatgccttctggggagtgtacctgccgggcggncgctc

SEQ ID NO: 51 - protein, 131 aa, Mus musculus
  misc_feature 1..2: unknown amino acid
XXGRGPRYSRRXSXKCKMAXMCAXRXQFYNSQTQICVGNPRERKSAFRGDSGGPLVCSNVAQGIVFYGSNDGNPSAVFTKIQSFMPWIKRTMRRLAPRYQRPANSLSQAQTXGLPXPLLMPSGECTCRAXA

SEQ ID NO: 52 - DNA, 248 bp, Mus musculus
  misc_feature 2..2: unknown nucleotide
tntttnggtcnnggccgngggactttnntttttttngttttttttttttngggggactagnaaaaaaactntaacgnaaacatgnggttcacgttacagtttggggngttctgggttttgatttctggggatctgacgaaggagcccanagtntnatacatgggaggcaagttctctccacacattgcaactggatgttcagcctctgactccttgnggagtaacatcacctgcccgggcggccgctc

SEQ ID NO: 53 - DNA, 207 bp, Mus musculus
  misc_feature 3..3: unknown nucleotide
tantttngncccnggccnngggnttcntngaaaccnnnantccagnnnangggntgccaannttttcntcagggncggaatctggcgttggaatttnactgncctgaagtcccaggatctnanaaactgnttggggcaattccttgcattggccatctgtagggaaattgcttctgctttacagagtacctgcccgggcggccgntc

SEQ ID NO: 54 - DNA, 866 bp, Mus musculus
  misc_feature 865..865: unknown nucleotide
tagcttgggtagagggcgaggtacattaaaatgggcagctaatattgcaaatggaccatatgcgcgccaatcgcttccagttctacaacagccggactcagatctgtgtgggaaattttccaagaaaagttatctaccttaatgggcgatactttgggccttctgcttccatattggtgtccgaaggtttttggttattaacaagttgcgttttaatacccgtccagttggtcaccggatggttgagttttttggtgccctccttgaaattcaccattgtacggcgaccattaactcatccaattagcttcgggctgggatgtatttaaaacagtatcttgtgcgttcctcctgctgtattaaaagggtttaactaatttttgagggtggtcttgaggtcctccaaaacacttattttcctagcaagtttcaaccatatccttgattgggtgcttttttttttagggtaactcattagaaatggtttgcattacttcatggaaatctaaaaatcttttggccactggttatggggtactaacctgggtaaaccttatgaaatacttaattgctctggttcattttttcataaaaacattactaatccccttgcattgagggtgttaataccttgattcctaactactttgtgcctgaactttctttattttgttttttgcacttcttttgctctctttttagatttgtttatgtttctttctgtccaacatttttttttttatgttacatatctctcttaggaaggcttcttttatgtttaggtactttttttctttgctctaaataaagtaactttcgcttaagtgatttgacgtgtgtgacactaaaatataaatatacatanc

SEQ ID NO: 55 - protein, 288 aa, Mus musculus
  misc_feature 9..9: unknown amino acid
AWVEGEVHXNGQLILQMDHMRANRFQFYNSRTQICVGNFPRKVIYLNGRYFGPSASILVSEGFWLLTSCVLIPVQLVTGWLSFLVPSLKFTIVRRPLTHPISFGLGCIXNSILCVPPAVLKGFNXFLRVVLRSSKTLIFLASFNHILDWVLFFLGXLIRNGLHYFMEIXKSFGHWLWGTNLGKPYEILNCSGSFFHKNITNPLALRVLIPXFLTTLCLNFLYFVFCTSFALFLDLFMFLSVQHFFFYVTYLSXEGFFYVXVLFFFALNKVTFAXVIXRVXHXNINIHX

SEQ ID NO: 56 - DNA, 576 bp, Mus musculus
  misc_feature 2..3: unknown nucleotide
tnntnggccgcccggccgggtaaattttattccaccgancccccagnttttanaatccctgggtaatgnagagcaggnccagataacgccccttaactcaagctgnatgtggtacctagcngaacagaaaagcacaaacgcacgagagaaacattccgagtgattactcacttgaactgagactcaagaaaggccccccagtcttctatccggtggaaganaacacttacattttcagggggaaaaaaaatgaacttgcagacagtcatgggaagccatagctgcacagagtatcgccaagtcttcaaaggacaatgaaagcaggcacgtgaccatggtgccaacaggacaggccacagatctgatcagtaagggtccaaagtcaaccttcacccccagggctggctaatgcttggtttaatttgaattttctttcttcttgagatttaaaattaatagatcctgaactggaagtatgtggaggttcaactctattggnagatgggtagatttggcacacatcccacattttcagatcaactaccacttgagggaagtccttgggcgggaccacct

SEQ ID NO: 57 - DNA, 686 bp, Mus musculus
  misc_feature 15..15: unknown nucleotide
tattttgggcgcggnccgagggacccnnnntttttttngtntttttttnnnnnnnnnnnnnnntnnnnnnnncntttntttttttnttttttttttttnnannnanaagnttttttnanaaaaaangggcaaancccncttttttttnaaaannaaaangnnaaanaaanggnaaatttttngggttttaaccntcnnaagntnggnccccccnntttnangnnnccnantttncntttttgaannncccttttanananncnccatttttttaantttgggtccaaattttttttttnttcccaantttgccnattccattgncaaanaaaaaaaaaatacccttcccgttttggaaaaaattnccccncccnttttcccgggnggnagtttaaaanggngggngggttnntgngcagacctgccccagcggcccccgggaggtcctggtnataaacaaccctncttacggntcccccgggaggacaagtgtttaattttggcctgttaacccttgctggggaaaaggggggggncccccnanggccttgttttanangtnctnggtnggggtggncccccaattcccttngggaaanaacccttttttttccttgggaattnaaacacccttgccaaaagggttnntttnaaccctttccgtttnttaagggngcccggggggc

SEQ ID NO: 58 - DNA, 797 bp, Mus musculus
  misc_feature 61..61: unknown nucleotide
tagcgtggtcgcggcccgagggtacgcttttcctgcataccatccctaaacacacatggcngangaaggtnatattttctaatttcttgtcctntgacatggatgccatggnactcacaggcctgcnacacaaaataaataagatagatgaatcatttaaaaaggtatntcctagataacatacagtgatntattaataaaataaacngtttggcctgtttatgaaatcaggataaaaccataaaagtaataaagaatagagaggtgtgtatgttagngagaaaaagacaaaaagcgttttatataagtcaataaaagttaaaattattggcaagagttnagtgaatgacaagaaataaannaancagtncaatatgagaatnatggttaaataatntatnantatanttantnngccctgggccngcnnaccacncnnaatggcngattttcanncacncntggcggtccgtttcntnnntggatnccngagtcngggtcccaaagcttcganngcnttanncttggaggttttctattnttggcnnccaaaatatgcgntgngnggtaatncattggnnnattggnttgtttnnnggggngaaatantgntnttccncctnacnntnttcnnncaatcaatncnancnngtaactatttnnggnatnaccncggggtggcnnnatatgtgagnctancctctntttatnnnnnttngtgctnnctcccctcttnntnctnnnnatacccttgttccnnnnnnnnataattaatttgntaccctccgngggaaagg

SEQ ID NO: 59 - DNA, 611 bp, Mus musculus
  misc_feature 2..3: unknown nucleotide
tnntntggtcgnngnccnaggnccagttnntggaaacctaaattagataatgactttcgccattcagtagactgtaaaggacctccctcaggtgggtcatggatgtgatttgaaagtcttagaaagctctttctaccccaaaataaattttaccgnggttcaagtttctttggttctagtcacaatacgactaccaagtgaagatgctttgtgaggaatttgnagttttaaaaattaatactttttatgttaaattactttatggttgtggggggttggttttgtttgtaggtattttattttttgcaaacttagtagaatagaagttactgtttatgtctagaaagaaaataagcttaatttgtctgtgttgggaggaagattgagctgggcttagatctgacattcatgnaatacactgtagttggaattattangataataaccttgtcactggtccagaacctggggaggatgctcattaaggaaagaggaagttatttctcaagangagtngctttttttttttggtggtgggttggattggggcncctacnactgtnggtggangcttntacaannnngagntcctggccgggaccngccggcgg

SEQ ID NO: 60 - DNA, 743 bp, Mus musculus
  misc_feature 7..7: unknown nucleotide
aaaaagnagaaagatgggaaagaccgttttggaaaaaaaccccgaaaagggntccgggaaaaaancanncncgagcggccnacagngggaaggaaancngcngaaatcnnccctantttgggnncgggccgngggnnccacnagggangggggggatttanaaagccaacnctnaaacncgggnnaaggnacnangggggaaagggcactnggnnggcncgaggagcncacanangnaannnagaggccnnnacccacggnancanaggnnnaaanctnggnngnaccgagccncgncagggnaggangcgangggggaaggggnaccnnngaanaccaancngnccaacagggnggnanccagnaggggacncaannannngaccactgggagcagggacacagncacngngngnagnncggngccacggcagnacccngntngagagacacncngacnacnnaacnnagcangagcgggnaaggcncnagccgngnncagnctanngaaagcnngcngagaccacanggggggncgcccncccaaggnancnaaanggggaaccgaannngnggaangnngcnccggaanggggcaacngnnaatngngaannngncgtnntnnaccccccgggnaannannaagnnccnanngngagngagggcggncccaagaccnnaannnnnacacgncgnaaactngatnnggnccaaggccaaaagggggggggaaccgganaaa

SEQ ID NO: 61 - DNA, 934 bp, Mus musculus
  misc_feature 7..7: unknown nucleotide
tattttngtcgcggccgaggtgcttgnttntnggcangngtngnntttgtnataggnggaagcaaagaccagcctngtacatacctatcttctaaccttngtgaaccnntgncgccaggcttgcattcttttaagctcaacaggtctgggtaaagtggcttccccttncttnaatnggcttgggttcattantgcctnccaactaaaaacccaaagncttcntcttcaatttcccattaagcangaacctaaagaagtgggcaanggggnnaaagngaagatataaccttctaacaagtaagtccnttcaatggtcttttctttggcaaaccagcntcngtacccttggcccccgggggccgggcccgaaaggggccgnaaattcccaagcnacaacctngggcngggcccggttnaccttaggtggggaatccccggagncttccgggtaccccaaaagccttgggatnggcaatnaagccttnggaaggtaatttctaataagtggtcnaccctaaaaataagccttgggcgtaaatccatnggggccaataagctngtttccctggngnggaaaaataggttaatcccgnttcacaaatttccacaccaacaataccgaaacccgggaaggcataaaaggngtaaaagccctggggggtgccctaatggaggngaagctaacctcacattaaattggcgttgcgctcactggcccggttttcangtcggggaaaaccgttgggccaactngattnatgaaacggcccaacccccgggaaaaggcggttgctanttggcncttttccgttctcgttaatgaataattgnnttggtcntnggntgnggcaaggggttaaatcatnaaggnggaaaccggtaacnccananggggaacccgnaaaanttggncaaagcccaaaggcggacccna

SEQ ID NO: 62 - DNA, 776 bp, Mus musculus
  misc_feature 9..9: unknown nucleotide
ttttttggncncggccgaggnncccnggntagggggcttttantgatagngttgannttngnnttccaggctgntnggaaattctgtctntcgtctnagtctcccaagtttggaanctacaggtgtnttccaccntatccaggggaagtntgctttntacatacagctgtanttgaaaacagctgcgctgggaaacactagtcactggttngtttaaccaagaattactgtatacttaaggaaaagcaacagtataaataaaaaagaccaagccaaacagatnaattgatcttagagaaattaagttgagtataacataaagaatttttttgtcccaaaatatncttcaaatatttgaggaattgctattactataaattaacaatggatttctataagaataggacaggtttttcngaagtaaactctcaaggtcgtgcaaacagcaactggcttaccactgactccncaaggcanagaatcttcccaaatcctgctcatacgacaaagagaaattatgtcatgtganggccatggacctccagacctaaggagagatctccatttacttctnaaaggttccaggcagntttaagtgagncctttaaccaaaagtcgttgggccanttngcttgttgggtaggggaattncctgccccggccggcccttttgnaanggccnnatttccaaccccctgggggccgtttctattgggntcngncttgggccccaaattggngctttntttggggtttttttttgggcccccananat

SEQ ID NO: 63 - DNA, 287 bp, Mus musculus
ttttttcgggcgcccgggcaggcaccatgcaaaatggggagaatttctggccggttgaaggggcacctgaatacggtcatggttccctctggacattgagggatcttgggcccggctgaacctagggtgggtgagaacccagtgcaactggggccccaggaccactagggaaacactgagggatgtcatactcactgcttgcctctaaactgaggactgagatcctcaacctctactgtggcagctagaaaaagagactgttaaggacctcggaccgcgaccacgct

SEQ ID NO: 64 - DNA, 363 bp, Mus musculus
  misc_feature 5..5: unknown nucleotide
gaganattcnatggttttgtgctgctcgagcggcgcngtgtgtggtatctgcagaattcgccttagcgnggncncnggggcnanggcnnttttttgnanccaanccanaagncccgnnnnccttggcctaaaggagggtgganagaacccacggnaagttctctagaggactatcaaaagggtccccaaaaagtaaacaggaatgtgccaggcaaagaagggaaaaatacagttgcagaaagcatgaggtctgagagaaaagactgacactgggggcttacagggagtgagtggaagagaggggtataagatgagcaaagagaaagaactgctcaagagtccgtacctgcccgggcggncgct

SEQ ID NO: 65 - DNA, 941 bp, Mus musculus
  misc_feature 4..4: unknown nucleotide
ttcnagcggccnccccgacaggcntttttactanncaaagnaananacgccngaccnccnggggcnattntggngnaaaaaaacccccccccagtttnaaaaagacggnntcnctnngggccctccccangaaacaaaannccagaccacccccaaacgggaaacacnganaccccnngngaacngannaaggnttctatttaancaaggcccncccncnnggggangccttnaccnagggancctaaatanggggcccccttggggccnncgnacccncgccnaaangggccaaaatatnccagacntanaactgggggggggnncngctnccanangggggaaatcccaancccgggnnccaaancctaggaaggcaatagcctnggangnattcnnntaangnggccccccnaaaatanccctggccntanaatnacggggncatnagcnngtttccccggngggaaaactggaaatcccccnncaccaaattccnacaacaaccanaccnaacccggnaagcataaaagaggaaaaanccccggngggggccccaaagaannggaaactaacccnaacaattaaantnangnngncgcccncnggcccccttttccaancgnngaaaanccgnccttgcccncccgganttanatanaaaccgcccnaaccccccgggggaaaangcccgnatcnggtaaatggggcanctnttncgctttccangnctaactggacnnaagangggcccggggaaattcagcnacgggcnaaacnggcttcancccancnnaaaaggnggggaanncaggggnttcccacaanatnanggggnaacnccncgaaaaaaaaangngggnnnaaaaggcccncncaaaanngcnngccccacaaaangnccgcggccngggcgggattaaaaanatacccccccccnt

SEQ ID NO: 66 - DNA, 95 bp, Mus musculus
  misc_feature 29..29: unknown nucleotide
ggtcgagaattcaaagccttttaatgcangctcgagcggncgccagnnnatccatatctgcagaattcncccttagcgnggncgcggcccnaggn

SEQ ID NO: 67 - DNA, 533 bp, Mus musculus
  misc_feature 14..14: unknown nucleotide
ttgaaactttgtanatgncgctcgagctttcgcanngtgancgatatctgcagaattcgccttnncgnggncccngggcnnnggcntttttcantgantttcnttattttcccggtgggaagggtggtgnttgntgnttnttcntcctccctttttggttccccttcccgtttggaaaccagggactttttatagcccagtatggctttnaactatgtaaactggtataggtaaaatgaccttgaactcccaactggttctaggaaaatgaaggcacctntcaccatgcctagctacagtcatcattggngtgttcctgttctgctctaggttcttcacccaggaagtttagctaattgctacgcaatttccccatttcccaatatgagtgcttgtgaaagtctccccagcactgctttagctttctacaagcaagtgtattggnattttggtcttcatactgntgaactcggtgcattaaaaactccgaacatttgctttttaaacatgctcatggtacctgcccgggcggc

SEQ ID NO: 68 - DNA, 304 bp, Mus musculus
  misc_feature 13..13: unknown nucleotide
gagattcattggntttaatgctgctcggcggncgcatgtgatggatatctgcagaattcgccctttcggggcccnggcnncggactttttttntnnccgggttcngacacntcanaagaggggnncaaatntacctttggntggntgggaancaccatntggntnctgggatttgaactttggaccttcngaagaacnggcnggtgctcttanccnctgnggcatcatatttncccanctntgatgcncattccnaaaaaantggggcgggcaaagagcttaacttnacctgccgggcgggcgg

SEQ ID NO: 69 - DNA, 658 bp, Mus musculus
  misc_feature 7..7: unknown nucleotide
atttcantggntttaatgctgctcagcggcggcnatgggnntgnttttngnanaatnngcccttnnangcggccgcccggcaggcntttttttttttttttttttnncctcctcctcctgntttttttttttttttttttaaaaaaagnctaaanttnggnaaangagggactncagaaanggctaagggttaanatctagttgggttcctggcntntttgtcatggggnctcacacttntntggggtntttgacactgggggcatttgatgccctcttttagcctntgcctgcacttcttagacncccnccccccncatcaaaataaattttaaaaataagaaaagttaggaggggctggggganacagctccatcagggaaaagngctttgccttgcanggcattgaaaaacccttaagttcaaattttccctggaagccccaccattaagccagggccagggtaagggcccggggggggggcnacaacaattttataaacccccaangggcctnggggnaccccttttgggggcntcaactggacctaagggaaaagcccttgccctaaattagggggggttcttatctaggctaaaggggagaaaagcccgccctcgaaaaaaacaaaccccgngtacnttggcccgaccacgc

SEQ ID NO: 70 - DNA, 593 bp, Mus musculus
  misc_feature 8..8: unknown nucleotide
atttcgantgcgntttaaatgctgctcgagcgggcngcaatggtgatnggttttttnnnnaaaattnncccttganncgggccccccgggccagggtcantttnccagggngccagttngggaaatccatctgctgnccgccccgtggcccggaaatgggcttttntccttggtagggctccccaagtccaaaatcagggccntgaagggtggtggggcanaagagtcctggggctatgctttcagnggaacgcatacagcangcaacaggatgggtgcagatgccaatgacacctccaaggatgagcgaagtcacgccgtttcctaaggttgatcctttgtatcaggctgttcacggcaggaaagcggttggccagagtgttcatcttgctgtgaattgacttgagcattcctctctgggaagtcatattctcttttgttgccatagcaatgcttattgtttcttctatcagacgatcagagtttcgaaggtggtcatgctctttcagaaacaagttccgttctcctgttgtttactccagacccgcttttatatgactcaatatctttccgcacctcggccgcgaccacgc

SEQ ID NO: 71 - protein, 197 aa, Mus musculus
  misc_feature 20..20: unknown amino acid
AWSRPRCGKILSHIKAGLEXTTGERNLFLKEHDHLRNSDRLIEETISIAMATKENMTSQRGMLKSIHSKMNTLANRFPAVNSLIQRINLRKRRDFAHPWRCHWHLHPSCCXLYAFXXKHSPRTLXPHHPSXPXFWTWGALPRXKAHFRATGRXADGFPXLAPWXXDPGPGGPXQGXIXXKKPITIAARSSSIXXAXE

SEQ ID NO: 72 - DNA, 919 bp, Mus musculus
  misc_feature 15..15: unknown nucleotide
aattttatcgttttnatgncggtcagcttcncatgtgnggnatntnncaaattcccctttgagnggnccgcccggnggggtntttttnananccttttacctcnaaggggccntnaggggggggnccccnnagggcccgggggggnatccctnggntttnnggcccggggggncaaaagttttgggnanttccnggntnttttgttaaaacggtttctctgtgccccnnagtngnggcccntacccttttaanantanaagangcggggnagtnnaanggaggccccctcattngtnntcccntgnggccncagttttttgngctggccntttgttcaggncaccaccgnaaangggccagncnttgatttnaaagtaaggttgggttaacccaangtttnggaaaacacacagactttggggccttttccaaaagaagagccnnggaaaaggtgncccattcaatccggattggggatccaatagtcaaaaccaaggttttaaaagcccaancttggaaccattcaaaagcccccaanttttnancaggccncangggncaagctttgnccttggnccttaaaagtaaggggaagatgatngggtntttgaaaattccgaaatttctcttgaattagaanagtcatcttttgnggncaacttaaggaagaggcanaaancccaggnccttactttgcttttaagnaaaaccaanaaaaagggtagggngggttaaacagaatctaacngntcatttttanttaaggggnttttttntnagaaaaaaaggganttttgggccntaantaancnggnggnnattcaaaacccnggaccaaaaaatnngctttttttttttttttccccnaaananaaagccccttttnaagggtttttttaaaaaggggaaaccnccccc

SEQ ID NO: 73 - DNA, 886 bp, Mus musculus
  misc_feature 19..19: unknown nucleotide
gccgggggccggatgggcntttnccttgggangngctccccaancccaaaatcanggccatgaangggggggggcaaaanagncctgggggctatgcctttcaagggggaaccgcntncggcnagcaacagggatgggngcanaatgcccanatngacncctccaanggatgaagccaagtcccgcccgntttccctaaagggtgggatccttttgggnatcaagggcctggttcaaccgggnaagggaaaaagcggggttggggcccaaggangtgggttcaaatccttngccttgggggaaattggggaccntttggaagnccaatttccccttctttctngggggnaaaagggcaaatnaatttccttcttttttggggttgggccccattaaggccaaaatnggcctttnaattgggggtttcccttttccttaatccaaaaaaccgnaatnccaaggaaggtttttcccgaaaaaaggggnggggnccaattggcctccttttttcaaaaaaaaccaaggttccccggntcctttcctgggttgggnttnaacctcccaanaacncccggntttttaatatggaacttcaaataatcttttcccgnaacctttgggccccggggaacccacgctaaagggccgaaatttcaacacactggcngggccggtacctattggggatnccgaacctccggnncccaacttgatgcatancttgagggattntattangggganccaaaaaacttggggggnaancangggnataagctgnttccngggggnaaaagggttccggtncaaatccacaaaaacaaccggaanatnaaagggaaaccngggggccaaaaaaaaaaaaaanaaatagggggnccccccc

SEQ ID NO: 74 - DNA, 848 bp, Mus musculus
  misc_feature 2..2: unknown nucleotide
gngtcanccggccccaagtgaatggantcngcaaaattccccttngagnggcccccgggccggannttttntatntacntcannncctgccaatnnngggggngaancncntnnnngcnggtttgagaccccntggggtnnnnnnnnttnngannanaggccctnaaatanggntgangcttangncatntnttccanccnnnactgaaattnntnttttcaccgggnctgacatgngncgtaggggagngtgggtgngngtttanccnnctcaaacgcccccgcttnggctggagccaaangctggcntggganggacccaaggcaagctcgggncagggctctggcatnncggganncttgggcttccnagtcttcacactngacccaaantnanagggaacctnaaaccaaggtgggtaananaactgacggcttcngatggaacaggaganacctgacacatcgaatgnaacntaggaactcggacggngaccacnctaanggngggaattcaggacactngncgggccnnactaaggggatnccgaacccngacccaacctttgatgcataaggttgaggattctnttgcgggacctaaataggntngggcttatccagggcattagttggttccctggggnaaatngtattccncttccattccccccaaaataccgaacccggaancatcaaggggnaaaaccccngggggcccctttngggggggcccacccncattntntnggggggggggcccccggcccgttttnnatgggggaaaacctnnngggccccnnttttttataanttcncccccccccgggggaggggntttntttttt

SEQ ID NO: 75 - DNA, 386 bp, Mus musculus
  misc_feature 13..14: unknown nucleotide
aaaaaaaattttnnaaaaaagcccttntggaaaaaagnncccctacgaaacnnttcgccaaaagtggaangcgcanatctggcagaaaaancgcccccctaaccgngggggcccccgggcccaaagggaaatnttttttggggccccaaaagaaaagggcccgaaaggcccnngggcggggntgaaattnaaaaaaaaanncccggggggcaaatntcnaaaaanccagggnttnccgaaaaagnngggnngnctcttgnaggcaaggntaaaaggggccnaanccttncgggggaaaaccagntnaaaaaacanggcnggcggacanggnccccttnangcatgagnatgaatntgcccccgngnacctgcccggncggccgttc

SEQ ID NO: 76 - DNA, 707 bp, Mus musculus
  misc_feature 3..3: unknown nucleotide
gcngngcacggtcccaagtgatggatatttncagaaattcccctaacnggggngccgncnaaggnncttttttgggggttncaanncccaaaaacccnnaactngggcaaaccccctnanaantnaaccttggggggtccncnngaaaacctancncatnngattcccccatantgaantaacctcccgccaacccttagggccctgtttacatgnggcaacngggggnggtntggtttccattcccctatcaacccatataagggccctgnatacttgatgggcaaggcnacttttgggcncaaatganggangngccttgctgaggtcttncacagactggtccangggcttgctcttntnttgcaccanaagccacactggctgagnnttacatgnaatatctgganatgcaacgcacggagtttggagactcctcccacaanggacacttaaaggagganacanagggacgngcaaagcaagacagatcttcnggagaagttgccatncctgnaggaagttggcatcattcatggntccnggttttggtcttgggtcttgagacaggctctcattttgtagatcaagctggcaaaactacanagctctcctggcttgccnccaaattgnaataaanacttnactgcccggcnggcgcccngcangtcttanacatgnctggaaattttngtaccggcttgggc

SEQ ID NO: 77 - protein, 235 aa, Mus musculus
  misc_feature 5..5: unknown amino acid
PSRYXNFQXCXRXAGRXPGSXVFIXIWXQARRAXXFCQLDLQNESLSQDPRPKPGXMNDANFLQXWQLLXKICLALXVPXXPPLSVXCGRSLQTPCVAXPDIXCXXQPVWLXVQXKSKPXDQSVXDLSKAXPSFXPKSXLAHQVXRALIWVDRGMETXPPPVAXCKQGPKGWREVXSXWGNXMXXVFXXTPQGXXXRGFAXVXGFWXLXPPKKXLXRXPXXGNFXKYPSLGTVXX

SEQ ID NO: 78 - DNA, 287 bp, Mus musculus
gcgcgaaaaatccagaaacacwgaggacgaaaattcaatagcgtttggtagatgcacgctcgagcggccgccagtgtgatggatatctgcagaattcggcccttaacgtgggcgcgggccgaggtacacacatgccgtgtggcatgcacatacacaggggcaggggggttcagcccataacaatgagtgaaatactggttttaacagaatggatgtgaatggacacatacgtatcacatagtctcatatatgagggccagaaatgtcaacctgcccgggcggccgct

SEQ ID NO: 79 - protein, 60 aa, Mus musculus
  misc_feature 14..14: unknown amino acid
RPPGQVDISGPHIXDYVIRMCPFTSILLKPVFHSLLWAEPPCPCVCACHTACVYLGPRPR

SEQ ID NO: 80 - DNA, 922 bp, Mus musculus
  misc_feature 2..2: unknown nucleotide
gnatkttwgarwacgattactatagggcgaattgggccctctagatgcatgctcgagcggcccgccagtgtgatggatatctgcagaattcgcccttattttgtcgcgggccgagggtacttctactaggctttaaaaaaaaggngtgcgttaacgggttctgactgccttaagtttccaaacccatgagtcacttatggtttgacctatgacaatccctgaaacgtgggtccctttgaccttccttctcccccatcaccagctgcttggctacattccccttctcaaagagctcacaagcgccggtccacgactcttaagtctctttctggaagtcatcacattatctgatatccatccacaagatggagttgtgtcatctactccatatgatactaacttagaagtctacaggctggacaagatgcttggtggttaaaagcctgctgctcttgcagtggatctgaatttggttgttcatggccgcgttgtttggagaggctcacaaccgctgcagccccagcaccactgtgacctgatacctgtggcttcttcttaggcttctgtcctcaaacacctgcccgggcgggcggttaagggcgaattccaagcacactgggcgggccggtactaagtgggatcccaagctcggtacccaagcttggatgcataagcttggaggttttctattaggggcaacctaaaaaagcttgggggtaatcatgggcataagtnggttcccgggggggaaaatgggnattccggttnacaatttccncacaaaaataccgaanccgggaagcnttaaaggggnaaaacccnggggggccctatggggggggccnaactcccatttaattggggggggggccaatgggccctttttaaaagggggaaaaccgtggggcccctttttttttaa

SEQ ID NO: 81 - protein, 274 aa, Mus musculus
  misc_feature 12..12: unknown amino acid
KKKGAPRFSPFXKGPIGPPPNXMGVXPPPXGPPGFXPFXASRXRYFCXEIVNRNXHFPPREPTYAHDYPQAFLGCPXXKTSKLMHPSLGTELGIPLSTGPPSVLGIRPXPPARAGVXGQKPKKKPQVSGHSGAGAAAVVSLSKQRGHEQPNSDPLQEQQAFNHQASCPACRLLSXYHMEXMTQLHLVDGYQIMXXLPERDLRVVDRRLXALXEGECSQAAGDGGEGRSKGPTFQGLSXVKPXVTHGFGNLRQSEPVNAXLFFKAXXKYPRPATK

SEQ ID NO: 82 - DNA, 360 bp, Mus musculus
tagcgtggtccgcggcccgaggtactttttgaaactgaaggacaacgagaagaaccggagctctttgatcttctcgatgtcctcgagttcaaccaggtggtgatctttgttgaagtccgtagcagcgctgcatcgccctggcccagcttctagtggaacagaacttcccagccattgctatccattgtggaatgccccaggaggagaggccctctcggtatcagcagttcaaggattttcagcggaggattcttgtggctaccaacctgtttggccgaggcatggatattgagcgtgtgaacattgctttcaactatgacatgccagaggactcggacacctacctgcccgggcggccgc

SEQ ID NO: 83 - protein, 71 aa, Mus musculus
  misc_feature 10..10: unknown amino acid
AWSAARGTFXNXRTTRRTGALXSSRCPRVQPGGDLCXSPXQRCIALAQLLVEQNFPAIAIHCGMPQEERPS

SEQ ID NO: 84 - DNA, 332 bp, Mus musculus
  misc_feature 98..98: unknown nucleotide
tctttcggccgcccgggcaggtacacacttaattttttcaagctattggaaaaggcttttaagcttaaccagcttcttccaccaccaaaaattcatanccatttnaanggggaatgaagttcttnaacatttttttctttggctgaataactaaaaanttngactaagcaaatgcctgaaagttgaagttatactnattatgtaatacattaagtagagacatgcaaatttttggggtaatctttttttttaatctgngattggctttcataattataatgagttagttttgcaatggaggttttactatgttacctcggccgcgaccacgc

SEQ ID NO: 85 - DNA, 255 bp, Mus musculus
  misc_feature 6..6: unknown nucleotide
ttgttnggccgcccgggcaggtactggatcatgtcgtaggtccngttcctgttgctgaggaaacagntctggatgaccttcgtgataaaattcgcagcctnccgctctgtcatgttcgggctgaacctgtgtttcaaaagtttgattgtctggccacgaaaacagggcaggcctgtgtccaacatgagtgtaaccagngngactactgcatccatatangggcgcacagncagataacctnggccgctaccacgc

SEQ ID NO: 86 - protein, 84 aa, Mus musculus
  misc_feature 5..5: unknown amino acid
RGSGXGYLXVRPYMDAVVXLVTLMLDTGLPCFRGQTIKLLKHRFSPNMTERXAANFITKVIQXCFLSNRNXTYDMIQYLPGRPN

SEQ ID NO: 87 - DNA, 610 bp, Mus musculus
  misc_feature 8..8: unknown nucleotide
cactcagngtcnatccgcagggtagaaggcggacagctntgnttcttnggtcaaagaaacnctgtntagatccctacgggngaaggtggttggnnnncgcaggtttntttccctgtagncctgaagctcaggctcttcagccttatgagaagtcaagaatcctaattttgtgttgggatgaggtactngagataacaacgtcccttggntgcgtttagncctgcntttccacatttaccctgtnntgccnnanttcctctcttgggtatgagaatcttttnattccagncgatggggnccagcttntcncattcanaaacctntttancttgtatnccaagnttntgtntctatcacannntcgtgcctgtttcctgtgtgaaattgntntncgtnacgtcctgcccggcggcccgcccggcangtntaaacatcanatnagcccaancttgnnaagcatctgccaaanatggatnaccaggacangcttcaccctnaangtgnataaaccactgaacctttnntgttnactgaaggngcggganacccaactnggaanggggggnaccaggctnantttnacttagggnctnctgggtaaaccccttgg

SEQ ID NO: 88 - DNA, 401 bp, Mus musculus
  misc_feature 2..2: unknown nucleotide
tnttttggtcgcggccgaggtactttnttttttttngngnnttttttttttnnacnaacnctattcaagggtcattttaaaacaacaaactcaggccaaacgggacaaatataaaattctcctntttgntcananaaccccacacatgngtngggtntatgcaagccaanatnggcttnttttaaaaggaaaatattgccctccataacactcaggnaganatcgtatccaaacnctngctgcaaaaatatgtcanactntatagatgaanacaggcttaaaaagagacnctanagtctgggaatcatcacaaagggccacanacttgttagncaatngnanatttcttcccttgacttanatagcacaccctccccgcgtacctgcccgggcggncgntc

SEQ ID NO: 89 - DNA, 639 bp, Mus musculus
  misc_feature 15..15: unknown nucleotide
tattttggtcgcggnccgaggtacactggaagctggctttaaganccaccagaaagagggagtcagatctcgttatggatggttgggagccaccatgtggttgccgggatttgaactcaggaccttcagaagagcagtcgggtgctcttacctgttgaaccatctcaccagccccggaaatgagattttnaactcttatgtaaatctgtccctaatctatccgacttgaggaagctttctttgccttcttttnggccaaaattctaccagagtgggttacttctacaattcatagaactttcccactctgacgaaactagttctgtctctaccaggagatagcagaagctccagaggaccaagaccacttgtcctggagtgtccaacaagccttcatcgaagcccgcgtactttnnnnnnnnntnnnnnnnnnnntnnaaancaaaacaccaggtcttggaacaaaaggcaggccaacatgaancattaanaaaacaagntcanagcaaattcanccggcanctgctgagaacttttgacccctnttccttaagggnctntnncaantggtttggntanaaataagggaanaaaagctttccgnaaccgngttaattttattaaaatttnacccngtac

SEQ ID NO: 90 - protein, 212 aa, Mus musculus
  misc_feature 3..3: unknown amino acid
TGXNFNKINXVXESFXSLIXXQTXXXXPLRXRGQKFSAXAGXICXXLVXLMXHVGLPFVPRPGVLXXXXXXXXXXSTRASMKACWTLQDKWSWSSGASAISWXRQNXFRQSGKVLXIVEVTHSGRILAXKKAKKASSSRIDXGQIYIRVXNLISGAGEMVQQVRAPDCSSEGPEFKSRQPHGGSQPSITRSDSLFLVXLKASFQCTSXRDQN

SEQ ID NO: 91 - DNA, 257 bp, Mus musculus
  misc_feature 3..3: unknown nucleotide
tantttccgcccgggcaggtggctgtctgtcactatggctgcccagttggggcaggctagcctatggggtgtgaagagttggccagagtcatgccgttctatggtggcaagcaaggagggtcagttcatatgcctctgctggcttttctatcgctaggggttgctagggggttgctagggatctacaccctgcctgtgggtgtanatggggctgcaagggctgaaaagttcccactgtacctcggccgcgaccacgc

SEQ ID NO: 92 - DNA, 499 bp, Mus musculus
  misc_feature 7..7: unknown nucleotide
tgttttngccgcccgggcaggtcctgaggtgtaagttggagtagattgtgccttctgaagcgatacctgtttacaatgaagttgcagtccccagaattccagtcccttttcaccgaaggattgaagagcctgacagaattatttgccaaagagaatcatgaattaagaatagcaggaggagcagtgagggatttactgaatggggtgaagcctcaagatgtggatttcgccaccactgccacccctactcagatgaaggagatgttccagtcagctggcattcgcatgatcaacaacaaaggagagaaacatgggactatcactgccaggcttcatgaagaaaattttgaagttactacactccgaattgatgttaccactgatggaagacatgcagaggtagaattcacaactgactggcagaaagacgctgaacgaagagatctcactataaattctatgtttctaggttttgatggtacctcggccgcgaccacgc

SEQ ID NO: 93 - protein, 166 aa, Mus musculus
  misc_feature 3..3: unknown amino acid
CFXRPGRSXGVSWSRLCLLKRYLFTMKLQSPEFQSLFTEGLKSLTELFAKENHELRIAGGAVRDLLNGVKPQDVDFATTATPTQMKEMFQSAGIRMINNKGEKHGTITARLHEENFEVTTLRIDVTTDGRHAEVEFTTDWQKDAERRDLTINSMFLGFDGTSAATT

SEQ ID NO: 94 - DNA, 711 bp, Mus musculus
  misc_feature 7..7: unknown nucleotide
tagtttngtcgcggccgaggtacggtgtatcaccacagtctgtgtcggctggtccagggaagccatcaattcttcgttaatgatcatcttgctgatgatgggagtgaacaagtggggtagatccagctcaaacatatntgataagtgtctccatactgattgagtcataaacactgctgnagggaaaaagggaagggcctcaaaaactctttctggaatcttccgaaactaccgtggtggcgaactttggcaagcctcaggggaaaaggtcccacacttcccattcatcttttcattaatgatgaaactgtggcaggtcttccagtcgcccatcttcatggccttggaggcagcggaccacatgctccctcattgactcgggaggacctagcaggggctgcccgntcgcccacccgcaagttggtggcggaactgcttgctgatcatgcgtcggcggcatcgctctcatggcagcatgtaggggatctncaggagcatagctgacaccagatagacacactccacanctcaggttgatgtgcaagtgaaangcacctgccggccggccgtcnaaaggcgaattcacacactgcggccgtctanggatcgactcggacactgtgcnnnctgagttcataggcacnatntgngaataggcanntgtctgggaatgttcgtaatcacaacacgactaaggancgggctaanaca

SEQ ID NO: 95 - protein, 237 aa, Mus musculus
  misc_feature 2..2: unknown amino acid
CXSPXLSRVVITNIPRXXPIXXXCLXTQXAQCPSRSXDGRSVXIRLXTAGRQVXFTCTSTXXVECVYLVSAMLLXIPYMLPXERCRRRMISKQFRHQLAGGRXGSPCXVLPSQXGSMWSAASKAMKMGDWKTCHSFIINEKMNGKCGTFSPEACQSSPPRXFRKIPERVFEALPFFPXAVFMTQSVWRHLSXMFELDLPHLFTPIISKMIINEELMASLDQPTQTVVIHRTSAATKL

SEQ ID NO: 96 - DNA, 276 bp, Mus musculus
  misc_feature 20..20: unknown nucleotide
tttttttcgcccgggcaggnacaccatccgcccccngggggtnnntggagtcgctggggagccccttcctgtggacagtgagcaggacatttttgattacatccagnggcgctaccgggagcccaaggatagaagtgaatgatgccctgccagccccaggcacagcccactaggagtcctaantnatttcttaacctttgctatgtaagggttttnggtgttcttaagcgatngcttcttctctgtgcttaccatgtacctcggccgcgaccacgc

SEQ ID NO: 97 - protein, 91 aa, Mus musculus
  misc_feature 7..7: unknown amino acid
FFRPGRXTIRPXGVXGVAGEPLPVDSEQDIFDYIQXRYREPKDRSEXCPASPRHSPLGVLXXFLTFAMXGFXVFLSDXFFSVLTMYLGRDH

SEQ ID NO: 98 - DNA, 419 bp, Mus musculus
  misc_feature 3..3: unknown nucleotide
tancnntggtcgcggccgaggnacgcggggcccaagcnngctgnnttctagcaacccttctgagcagagaanataaggggnnnaacataacacccgggaaagggcnntcntctcaaagccangnncaccaggaaaannaaaacaatnacataanataacccnaccttnnnngaacagcaancngcagctaccagaaaacctcctcanganatnnncaagaactaaggcccggnccggacatgctctntctcttctagncacactnccacgacataggggacaanggatnccacntnctctcattgcaggcaggaaacatnnnctacagaaactaagccaaacatnngggacagggnaatnccnaaaaaaaaaananaaanaaaaaanggncctgcccgggcgggcggccgggcagggac

SEQ ID NO: 99 - protein, 139 aa, Mus musculus
  misc_feature 10..11: unknown amino acid
PCPAARPGRXXFXXXFFFXXXPVPXVWLSFCXXCFLPAMRXXGIXCPLCRGSVXRRXRACPXRALVLXXXXRRFSGSCXLLFXXGXVXLCXCFXFPGXXGFEXXALSRVLCXXPYXLCSEGLLEXSXLGPRVPRPRPXX

SEQ ID NO: 100 - DNA, 205 bp, Mus musculus
  misc_feature 9..9: unknown nucleotide
gcgttatcngtttaaacncctcctgananatacnncgaaattggggntcctctagaatgcatngctcggagcgggccgccagtagtgggatgggnataatctggccagtaaatntccgccctttantntttttgttcgcngggcnccggaggggnccgggcnccgaggggtaacctggggggggggnntttttttttaaaaaaaa

SEQ ID NO: 101 - DNA, 752 bp, Mus musculus
  misc_feature 78..78: unknown nucleotide
tattttggtcgcggccgaggtgtagtcattttattttcaattacttggtattgtggtggtgcccctgtggtggatatngtgcatgtgtgaagctgctgcccagggtggtcctgaacaaggttgttagaactgctgagttgcaactatagacagttatatgctgncctacatgggtgctaggaactgaattcaagccctattcaagaacaagtacctgcccgggcggccgctcgattgggcgctcttccgcttcctcgctcactgactcgctgcgctcggtcgttcggctgcggcgagcggtatcagctcactcaaaggcggnaatacggttatccacagaatcaggggataacgcaggaaagaacatgtgaacaaaaggccagcaaaaggccaggaaccgtaaaaaggccgcgttgctggcgtttttccataggctccgccccctgacgagcatacaaaaatcgacgctcaagtcaaaagtggcgaaacccgacaggactataaagataccangccgtttccccctggaagctcctngngcgctnttctgttccgacctgccgtttaccggatcctgnccnnctttntcctttnggaaacgnggggcttttttnanantaaccntgaaggattnnaantcnggggnaggncnntcnnttcaaactggggttggggcaccaaacccccgttaaaccccanccntggcccttancggnanatttggntgnagccaacccggnaaaaacaaatta

SEQ ID NO: 102 - protein, 250 aa, Mus musculus
  misc_feature 26..26: unknown amino acid
FWSRPRCSHFIFNYLVLWWCPCGGYXACVKLLPRVVLNKVVRTAELQLXTVICXPTWVLGTEFKPYSRTSTCPGGRSIGRSSASSLTDSLRSVVRLRRAVSAHSKAXIRLSTESGDNAGKNMXTKGQQKARNRKKAALLAFFHRLRPLTSIQKSTLKSKVAKPDRTIKIPXRFPLEAPXALFCSDLPFTGSXPXXSFXKRGAFXXXTXKDXXSGXGXSXQTGVGAPNPRXTPXXALXGXFGXSQPGKNKL

SEQ ID NO: 103 - DNA, 180 bp, Mus musculus
  misc_feature 7..7: unknown nucleotide
ttttttngtcgcggncgagggancnggnngtccgccaaggaaggtncgnaaaancancgccctgaacgaagacctgaaaacgtggacggcggcggacatggcggcncanatcacccgacgcaagtgggagcaaagtggtgctgcagagcatacaaggcctacctgcccgggcggccgntc

SEQ ID NO: 104 - protein, 59 aa, Mus musculus
  misc_feature 2..2: unknown amino acid
FXVAXEGXGXPPRKVRKXXALNEDLKTWTAADMAAXITRRKWEQSGAAEHTRPTCPGGR

SEQ ID NO: 105 - DNA, 398 bp, Mus musculus
  misc_feature 4..4: unknown nucleotide
tatnttngtcgcgggccganggcaaaggggggaggctngggaaaaanttaaaaaaggggaantgcggggncttgaggntnagctgggangggcaaantnctgaaaggacaaataaacnaaaangncccaatgaaaanaaagctggnncctncccctgngaaggccnggnanctnnanggnaaggcccggcccgngcgnccccngcngnnncaccanncaaacanangtnganagggcccaccnacctactnannanactttngaccanngncactnaaatggnggaaaaaggcaggaaggncaaagcnagaacnntaaaggcnannacaggtacaannatnccngnnaaagggaaccnnacacccaancnaaagggaganaccagganaaagaaaccc

SEQ ID NO: 106 - DNA, 376 bp, Mus musculus
  misc_feature 2..2: unknown nucleotide
tntttttngtcgcggcccaaggtgaaaaggcccaaaagnnagnggtcttncaaaggggggaatccaggcctgtggccccacccaaggtcttggcaagggactnggaaaaaggaggaaagggttctggccaaaatgtaaacaatgctggctccttcctgctgngaaggcctgggatctccaggggaaaagctgggtatcaaccagcagctntcagngacttnacaaacagaggaagagagtggcactcaccgacttctttctcctggaccntcttacattctagtggccctnaccctnctcccctttcccaagctggactctntnaatcccagacaggtccaggggatacccgcgtacctgcccgggcggccgttcg

SEQ ID NO: 107 - protein, 125 aa, Mus musculus
  misc_feature 1..1: unknown amino acid
XFXSRPKVKRPKXXXSXKGGNPGLWPHPRSWQGTXKKEERVLAKMXTMLAPSCXEGLGSPGEKLGINQQLSXTXQTEEESGTHRLLSPGPSYILVALTLLPFPKLDSXNPRQVQGIPAYLPGRPF

SEQ ID NO: 108 - DNA, 261 bp, Mus musculus
  misc_feature 2..2: unknown nucleotide
tnttttngtcgcggnccgaggaccccgggccctttcttacgccttttnggggatgtatcaactggaataagacagaaatgacagagcccggctngaaaaaaatgtnctttcagctcatagctacgatggagactagcttctctccctgcagacacttgaaactttaaacactgctcagcgncattcatgaaatctcattctcaaaaccacaggtctgagaccaaccacnggaacaactcatacctggcccngggcggccgt

SEQ ID NO: 109 - DNA, 478 bp, Mus musculus
  misc_feature 6..8: unknown nucleotide
tttcannnggccgcccggcnaggggntttttgaaatanngccnnnggttgggnngntgggggggactncncaataaaancagccatgaantnctttttttaangagngttcngaaaangaacacttacctaaancttgaaagctataaactagnnatccatactntcatgacagtttagcnggagaacaacaacaaaaagatctatccctttaaaatatatttgggcanaaactgcatgtaatcctgagtttcctcctgacatacatatgttnggggaagtattctactctatacttgccaacggnggagaacaaaataagcttttnngagcgaagaagtntntntatatancnataaatanaatatccatcctggattaaatgngcaagcanagatcaccttctgnngggntcttcccgcgggngnncacacccacagacngtgcntgcgctgatcctgtatgactaangcgngtcc

SEQ ID NO: 110 - DNA, 870 bp, Mus musculus
  misc_feature 740..740: unknown nucleotide
gtcgatttagaacctttactatagggcgaattgggccctctagatgcatgctcgagcggccgccagtgtgatggatatctgcagaattcgcccttagtttggtcgcggccgaggttgctttggaaatactggcccggttaaatttattcatactttagataggatttgcatgcgctgatgaaattttaaataccatttcaaaacgctcgcaggagaacggaaatgcgatgaagtgggagagaggggtgataccgttgatgacagcagagtagacatccggaaaactgggtcatttacacgagagctgcagcctcccctgctaaaacacgccaatgacagtatgaaaaatggtttgtgattttaaagaaatatagcactccaaatgtcagcgtgagagaattcaataacacttaatgaaactttggatttcaaattaattactgattgactacagcatcaggggagaaaacagcctcgattagaggagctggggcaggctataaatccggacagctctttccaggacacggagctgggacttcctgctcaggtcaatgaggcccggttaatggggttacagatctcttaaatggccactgattggattggcagacataagacttttcctgacgtggctgggcctttgctcttttccttgttcctgactttaagaacatttaaacttggattggtggttcccaaaactcgcttaccggagcgtgaaaaaataagcgtctaanggnggttttaaaggtggnnntttnnttaaaaaaacctggcggacgccctgggggaatttaaccccgggggggttttgggtccgccggcccccttggcaatggttttttggccaaaatgggggaagggaag

SEQ ID NO: 111 - DNA, 175 bp, Mus musculus
  misc_feature 12..12: unknown nucleotide
gatgctgggcanaattcccccaacactgnttccatgccatgaaatgcacatggagcctggcctggcaaggcacagggngcagctnagcctttgggtcctgnggctcgcatttgaatcctagcttaccccctntgggttntgtgaccaaggaaagtcacctnggccgsgaccacnc

(19) United States
(12) Patent Application Publication
(10) Pub. No.: US 2005/0130138 A1
(43) Pub. Date: Jun. 16, 2005

(54) Immune-related proteins and the regulation of the same

(75) Inventor: Jeffrey Encinas, Nara (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/477,445

(22) PCT Filed: May 10, 2002
(86) PCT No.: PCT/EP02/05127

Related U.S. Application Data
(60) Provisional application No. 60/290,312, filed on May 11, 2001.

(51) Int. Cl. C12Q 1/68; C07H 21/04; C07K 14/74; C07K 16/28

(52) U.S. Cl. 435/6; 435/69.1; 435/320.1; 435/325; 530/350; 530/388.22; 536/23.2

(57) ABSTRACT

Disclosed are novel nucleic acid and amino acid sequences of immune-related proteins. Reagents that bind to immune-related gene products can be used to treat conditions involving inflammatory processes, such as allergy, asthma, autoimmune diseases, and other chronic inflammatory diseases where an over-activation or prolongation of the activation of the immune system causes damage to tissues.

### TECHNICAL FIELD OF THE INVENTION

The present invention relates to nucleic acid and amino acid sequences of novel immune-related proteins and their use in diagnosis and therapy for diseases. More specifically, the present invention relates to nucleic acid and amino acid sequences of proteins which are activated by IL-4 and/or turned off upon stimulation by IL-4 and their use in diagnosis and therapy for diseases.

### BACKGROUND OF THE INVENTION

Signal Transducer and Activator of Transcription (Stat) is one of the families of transcription factors, which play a major role in cellular function by inducing the transcription of specific mRNAs. Transcription factors, in turn, are controlled distinct signaling molecules. There are seven known mammalian Stat family members. The recent discovery of Drosophila and Dictyostelium discoideum Stat proteins suggest that Stat proteins have played an important role in signal transduction since the early stages of our evolution [Yan R. et al., Cell 84:421-430 (1996); Kawata et al., Cell 89:909 (1997)]. Stat proteins mediate the action of a large group of signaling molecules including the cytokines and growth factors (Darnell et al. WO 95/08629, 1995).

Stat6 is a component of the interleukin-4 (IL-4) signaling pathway that is activated by tyrosine phosphorylation upon the binding of IL-4 to the IL-4 receptor. Activation of Stat6 induces a variety of cellular functions including mitogenesis, T-helper cell differentiation, and immunoglobulin isotype switching. Mice in which the Stat6 gene has been disrupted show no proliferation of B cells in response to stimulation with IL-4 and anti-IgM antibody, no increase in expression of CD23 (FcεRII) and MHC class II molecules in B cells in response to IL-4, a reduction in T cell proliferative responses, and reduced production of Th2 cytokines and IgE and IgG1 after nematode infection. Although the IL-4 receptor is also known to employ at least one other signaling molecule in addition to Stat6, named 4PS, Stat6 appears to be essential for most of the known signaling functions downstream of the IL-4 receptor.

Since IL-4 plays a major role in many immune disorders, such as allergy, atopy, and asthma, there is a need in the art to identify novel proteins which is activated by IL-4 and/or turned off upon stimulation by IL-4 to provide therapeutic effects, particularly for diseases and conditions involving immunologically-mediated responses.

### SUMMARY OF THE INVENTION

The present invention provides polynucleotides which have been identified as novel immune-related proteins. The polynucleotide of the present invention is selected from the group consisting of; a) a polynucleotide encoding a protein that comprises the amino acid sequence of any one of SEQ ID NOs: 5, 19, 21, 23, 26, 28, 30, 33, 38, 44, 51, 55, 71, 77, 79, 81, 83, 86, 90, 93, 95, 97, 99, 102, 104, and 107; b) a polynucleotide comprising the sequence of any one of SEQ ID NOs:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111; c) a polynucleotide which hybridizes under stringent conditions to a polynucleotide specified in (a) and (b); d) a polynucleotide the nucleic acid sequence of which deviates from the nucleic acid sequences specified in (a) to (c) due to the degeneration of the genetic code; and e) a polynucleotide, which represents a fragment, derivative or allelic variation of a nucleic acid sequence specified in (a) to (d).

The present invention also provides an expression vector including the above-mentioned polynucleotide, host cell containing the expression vector, and protein encoded by the above-mentioned polynucleotide.

Further, the present invention provides a method for producing a polypeptide. The method of the present invention includes: a) culturing the host cell under conditions suitable for the expression of the polypeptide; and b) recovering the polypeptide from the host cell culture.

The present invention also provides a method for the detection of immune-related polynucleotides in a biological sample. The method comprises the steps of: a) hybridizing any polynucleotide of above-identified to nucleic acid material of a biological sample, thereby forming a hybridization complex; and b) detecting said hybridization complex.

Another embodiment of the present invention provides a method for the detection of the above-mentioned polynucleotide or the above-mentioned protein. The method comprises a) contacting a biological sample with a reagent that specifically interacts with the above-mentioned polynucleotide or the above-mentioned protein, and detecting the interaction.

Yet another embodiment of the present invention provides a diagnostic kit for conducting the above-mentioned method.

Further embodiment of the present invention provides a method of screening for agents which regulate (decrease or increase) the activity of immune-related polypeptides of the present invention. The method comprises the steps of: contacting a test compound with a polypeptide encoded by any of the above-mentioned polynucleotides and detecting binding of the test compound to the polypeptide, wherein a test compound which binds to the polypeptide is identified as a potential therapeutic agent for regulating the activity of immune-related polypeptides.

Yet another method of screening for agents which regulate the activity of immune-related polypeptides comprises the steps of: contacting a test compound with any of the above-mentioned polynucleotide and detecting binding of the test compound to any of the above-mentioned polynucleotide, wherein a test compound which binds to the polynucleotide is identified as a potential therapeutic agent for regulating the activity of immune-related polypeptides.

Further, the present invention provides a method of modulating (reducing or increasing) the activity of immune-related polypeptides of the present invention. The method comprises the step of: contacting a cell with a reagent that specifically binds to any of the above-mentioned polynucleotide or the above-mentioned protein, whereby the activity of the immune-related polypeptide is modulated (reduced or increased).

Another embodiment of the present invention provides a purified reagent that modulates the activity of the immune-related polypeptide or polynucleotide, wherein said reagent is identified by any of the above-mentioned method.

Yet another embodiment of the present invention provides a pharmaceutical composition. The composition includes a reagent which modulates the activity of the immune-related polypeptide or polynucleotide; or the above mentioned expression vector; and a pharmaceutically acceptable carrier.

Further embodiment of the present invention provides a use of the above-mentioned expression vector or the above-mentioned reagent in the preparation of medicament for modulating the activity of the immune-related protein in a diseases.

Further embodiment of the present invention provides a method for treating immunologically mediated condition. The method comprises administering to a subject in need of such treatment an effective amount of the reagent or the pharmaceutical composition.

### BRIEF DESCRIPTION OF THE DRAWING

FIG. 1 shows the DNA-sequence encoding a immune related polypeptide (WTT).

FIG. 2 shows the DNA-sequence encoding a immune related polypeptide (WTT).

FIG. 3 shows the DNA-sequence encoding a immune related polypeptide (WTB).

FIG. 4 shows the DNA-sequence encoding a immune related polypeptide (WTB).

FIG. 5 shows the amino acid sequence deduced from the DNA-sequence of FIG. 4.

FIG. 6 shows the DNA-sequence encoding a immune related polypeptide (WTB).

FIG. 7 shows the DNA-sequence encoding a immune related polypeptide (WTB).

FIG. 8 shows the DNA-sequence encoding a immune related polypeptide (WTB).

FIG. 9 shows the DNA-sequence encoding a immune related polypeptide (WTB).

FIG. 10 shows the DNA-sequence encoding a immune related polypeptide (WTB).

FIG. 11 shows the DNA-sequence encoding a immune related polypeptide (WTB).

FIG. 12 shows the DNA-sequence encoding a immune related polypeptide (WTB).

FIG. 13 shows the DNA-sequence encoding a immune related polypeptide (WTB).

FIG. 14 shows the DNA-sequence encoding a immune related polypeptide (WTB).

FIG. 15 shows the DNA-sequence encoding a immune related polypeptide (WTB).

FIG. 16 shows the DNA-sequence encoding a immune related polypeptide (WTB).

FIG. 17 shows the DNA-sequence encoding a immune related polypeptide (WTB).

FIG. 18 shows the DNA-sequence encoding a immune related polypeptide (WTB).

FIG. 19 shows the amino acid sequence deduced from the DNA-sequence of FIG. 18.

FIG. 20 shows the DNA-sequence encoding a immune related polypeptide (WTB).

FIG. 21 shows the amino acid sequence deduced from the DNA-sequence of FIG. 20.

FIG. 22 shows the DNA-sequence encoding a immune related polypeptide (WTB).

FIG. 23 shows the amino acid sequence deduced from the DNA-sequence of FIG. 22.

FIG. 24 shows the DNA-sequence encoding a immune related polypeptide (WTB).

FIG. 25 shows the DNA-sequence encoding a immune related polypeptide (WTB).

FIG. 26 shows the amino acid sequence deduced from the DNA-sequence of FIG. 25.

FIG. 27 shows the DNA-sequence encoding a immune related polypeptide (WTB).

FIG. 28 shows the amino acid sequence deduced from the DNA-sequence of FIG. 27.

FIG. 29 shows the DNA-sequence encoding a immune related polypeptide (WTB).

FIG. 30 shows the amino acid sequence deduced from the DNA-sequence of FIG. 29.

FIG. 31 shows the DNA-sequence encoding a immune related polypeptide (WTB).

FIG. 32 shows the DNA-sequence encoding a immune related polypeptide (WTB).

FIG. 33 shows the amino acid sequence deduced from the DNA-sequence of FIG. 32.

FIG. 34 shows the DNA-sequence encoding a immune related polypeptide (WTB).

FIG. 35 shows the DNA-sequence encoding a immune related polypeptide (WTB).

FIG. 36 shows the DNA-sequence encoding a immune related polypeptide (WTB).

FIG. 37 shows the DNA-sequence encoding a immune related polypeptide (WTB).

FIG. 38 shows the amino acid sequence deduced from the DNA-sequence of FIG. 37.

FIG. 39 shows the DNA-sequence encoding a immune related polypeptide (WTB).

FIG. 40 shows the DNA-sequence encoding a immune related polypeptide (WTB).

FIG. 41 shows the DNA-sequence encoding a immune related polypeptide (WTB).

FIG. 42 shows the DNA-sequence encoding a immune related polypeptide (WTB).

FIG. 43 shows the DNA-sequence encoding a immune related polypeptide (KOT-B).

FIG. 44 shows the amino acid sequence deduced from the DNA-sequence of FIG. 43.

FIG. 45 shows the DNA-sequence encoding a immune related polypeptide (KOT).

FIG. 46 shows the DNA-sequence encoding a immune related polypeptide (KOT).

FIG. 47 shows the DNA-sequence encoding a immune related polypeptide (KOT).

FIG. 48 shows the DNA-sequence encoding a immune related polypeptide (KOT).

FIG. 49 shows the DNA-sequence encoding a immune related polypeptide (KOT).

FIG. 50 shows the DNA-sequence encoding a immune related polypeptide (KOT).

FIG. 51 shows the amino acid sequence deduced from the DNA-sequence of FIG. 50.

FIG. 52 shows the DNA-sequence encoding a immune related polypeptide (KOT).

FIG. 53 shows the DNA-sequence encoding a immune related polypeptide (KOT).

FIG. 54 shows the DNA-sequence encoding a immune related polypeptide (KOT).

FIG. 55 shows the amino acid sequence deduced from the DNA-sequence of FIG. 54.

FIG. 56 shows the DNA-sequence encoding a immune related polypeptide (KOT).

FIG. 57 shows the DNA-sequence encoding a immune related polypeptide (KOT).

FIG. 58 shows the DNA-sequence encoding a immune related polypeptide (KOT).

FIG. 59 shows the DNA-sequence encoding a immune related polypeptide (KOT).

FIG. 60 shows the DNA-sequence encoding a immune related polypeptide (KOT).

FIG. 61 shows the DNA-sequence encoding a immune related polypeptide (KOT).

FIG. 62 shows the DNA-sequence encoding a immune related polypeptide (KOT).

FIG. 63 shows the DNA-sequence encoding a immune related polypeptide (KOT).

FIG. 64 shows the DNA-sequence encoding a immune related polypeptide (KOT).

FIG. 65 shows the DNA-sequence encoding a immune related polypeptide (KOT).

FIG. 66 shows the DNA-sequence encoding a immune related polypeptide (KOT).

FIG. 67 shows the DNA-sequence encoding a immune related polypeptide (KOT).

FIG. 68 shows the DNA-sequence encoding a immune related polypeptide (KOT).

FIG. 69 shows the DNA-sequence encoding a immune related polypeptide (KOT).

FIG. 70 shows the DNA-sequence encoding a immune related polypeptide (KOT).

FIG. 71 shows the amino acid sequence deduced from the DNA-sequence of FIG. 70.

FIG. 72 shows the DNA-sequence encoding a immune related polypeptide (KOT).

FIG. 73 shows the DNA-sequence encoding a immune related polypeptide (KOT).

FIG. 74 shows the DNA-sequence encoding a immune related polypeptide (KOT).

FIG. 75 shows the DNA-sequence encoding a immune related polypeptide (KOT).

FIG. 76 shows the DNA-sequence encoding a immune related polypeptide (KOT).

FIG. 77 shows the amino acid sequence deduced from the DNA-sequence of FIG. 76.

FIG. 78 shows the DNA-sequence encoding a immune related polypeptide (KOT).

FIG. 79 shows the amino acid sequence deduced from the DNA-sequence of FIG. 78.

FIG. 80 shows the DNA-sequence encoding a immune related polypeptide (KOT).

FIG. 81 shows the amino acid sequence deduced from the DNA-sequence of FIG. 80.

FIG. 82 shows the DNA-sequence encoding a immune related polypeptide (KOT).

FIG. 83 shows the amino acid sequence deduced from the DNA-sequence of FIG. 82.

FIG. 84 shows the DNA-sequence encoding a immune related polypeptide (KOB).

FIG. 85 shows the DNA-sequence encoding a immune related polypeptide (KOB).

FIG. 86 shows the amino acid sequence deduced from the DNA-sequence of FIG. 85.

FIG. 87 shows the DNA-sequence encoding a immune related polypeptide (KOB).

FIG. 88 shows the DNA-sequence encoding a immune related polypeptide (KOB).

FIG. 89 shows the DNA-sequence encoding a immune related polypeptide (KOB).

FIG. 90 shows the amino acid sequence deduced from the DNA-sequence of FIG. 89.

FIG. 91 shows the DNA-sequence encoding a immune related polypeptide (KOB).

FIG. 92 shows the DNA-sequence encoding a immune related polypeptide (KOB).

FIG. 93 shows the amino acid sequence deduced from the DNA-sequence of FIG. 92.

FIG. 94 shows the DNA-sequence encoding a immune related polypeptide (KOB).

FIG. 95 shows the amino acid sequence deduced from the DNA-sequence of FIG. 94.

FIG. 96 shows the DNA-sequence encoding a immune related polypeptide (KOB).

FIG. 97 shows the amino acid sequence deduced from the DNA-sequence of FIG. 96.

FIG. 98 shows the DNA-sequence encoding a immune related polypeptide (KOB).

FIG. 99 shows the amino acid sequence deduced from the DNA-sequence of FIG. 98.

FIG. 100 shows the DNA-sequence encoding a immune related polypeptide (KOB).

FIG. 101 shows the DNA-sequence encoding a immune related polypeptide (KOB).

FIG. 102 shows the amino acid sequence deduced from the DNA-sequence of FIG. 101.

FIG. 103 shows the DNA-sequence encoding a immune related polypeptide (KOB).

FIG. 104 shows the amino acid sequence deduced from the DNA-sequence of FIG. 103.

FIG. 105 shows the DNA-sequence encoding a immune related polypeptide (KOB).

FIG. 106 shows the DNA-sequence encoding a immune related polypeptide (KOB).

FIG. 107 shows the amino acid sequence deduced from the DNA-sequence of FIG. 106.

FIG. 108 shows the DNA-sequence encoding a immune related polypeptide (KOB).

FIG. 109 shows the DNA-sequence encoding a immune related polypeptide (KOB).

FIG. 110 shows the DNA-sequence encoding a immune related polypeptide (KOB).

FIG. 111 shows the DNA-sequence encoding a immune related polypeptide (KOB).

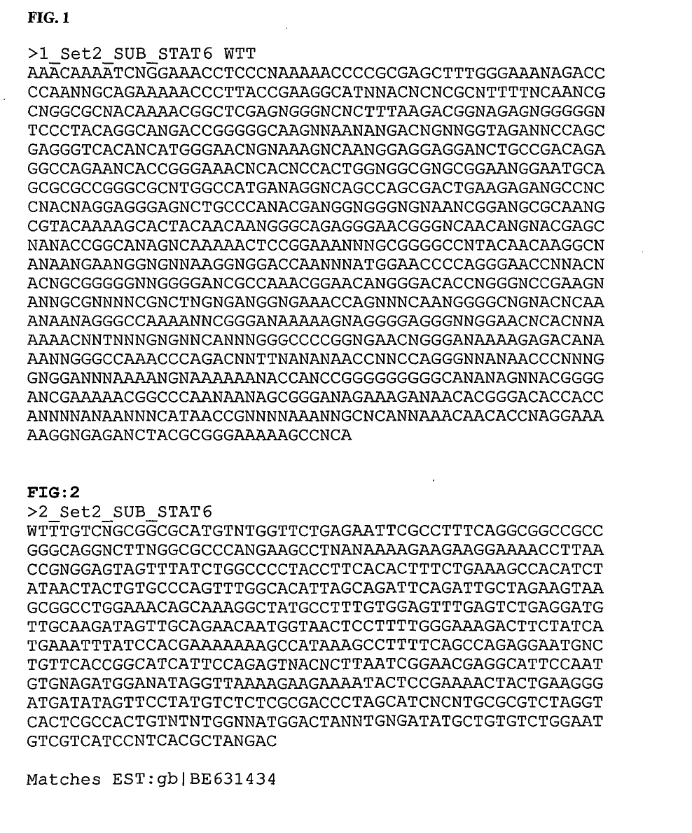

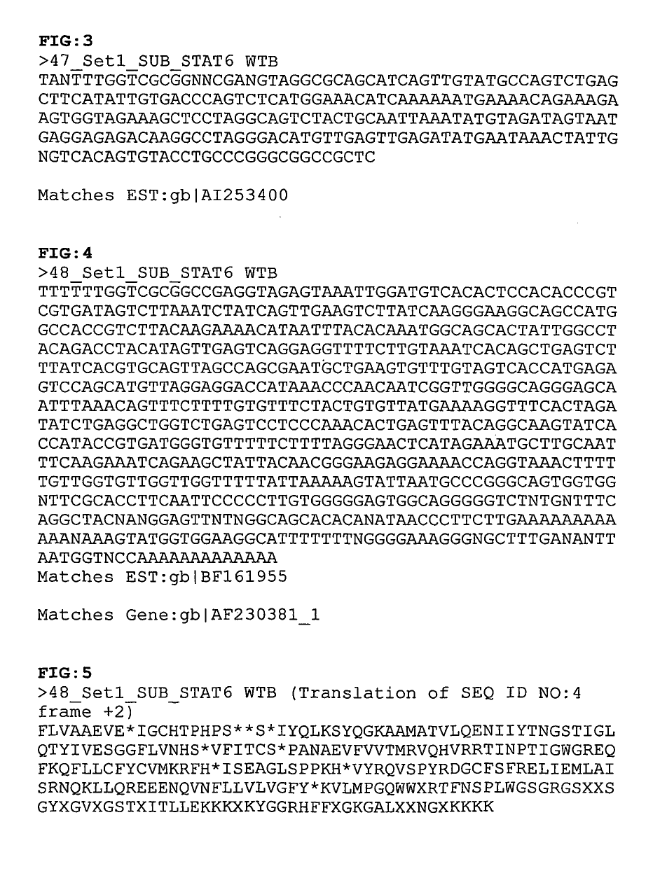

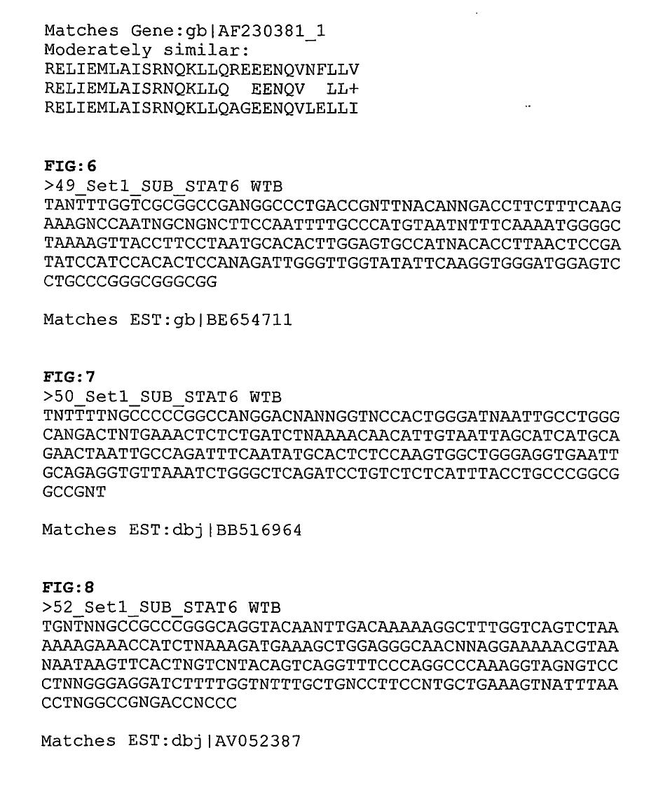

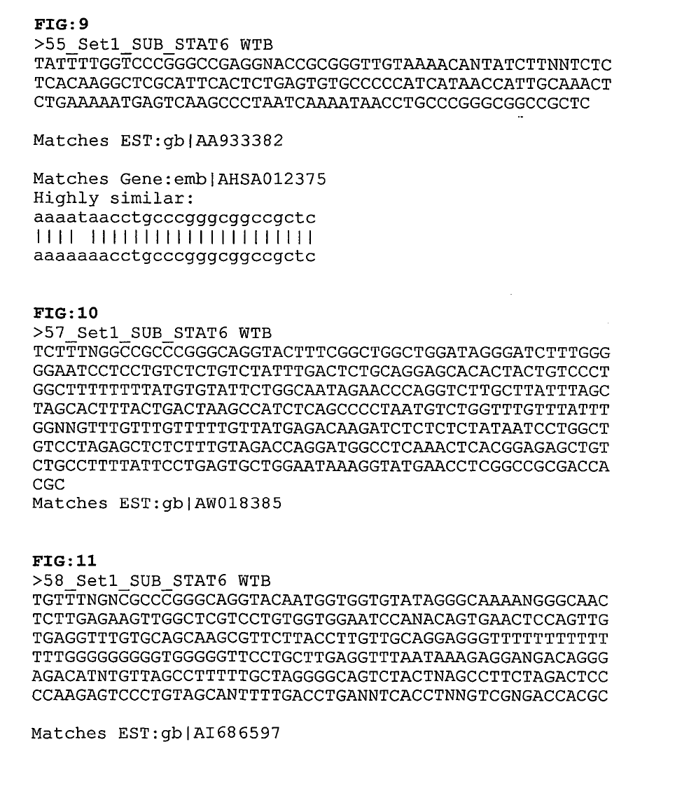

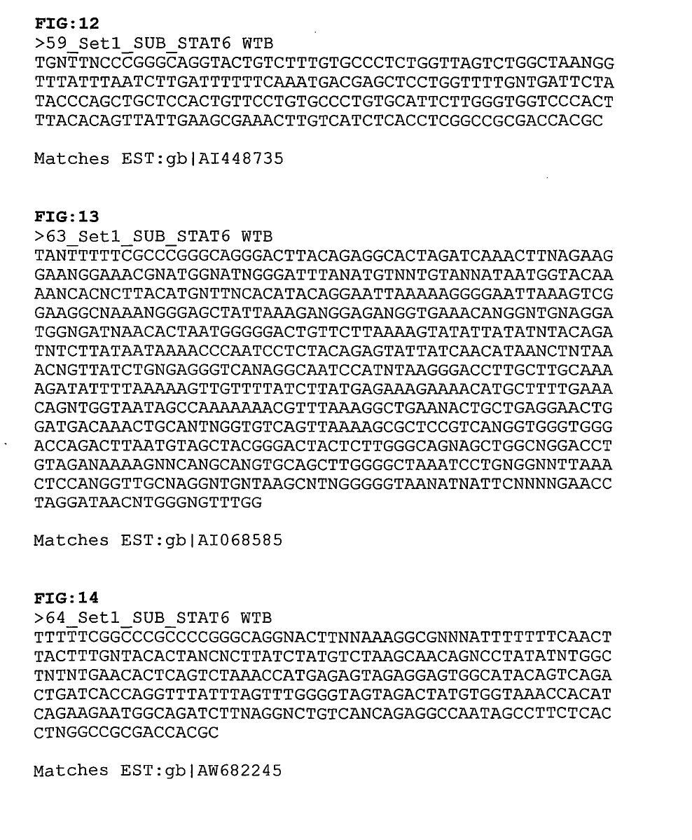

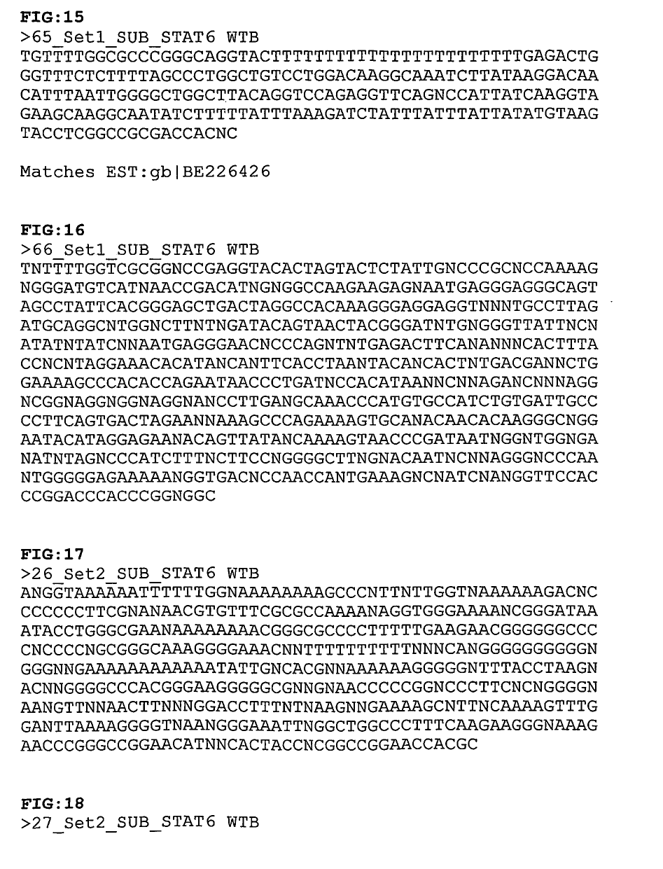

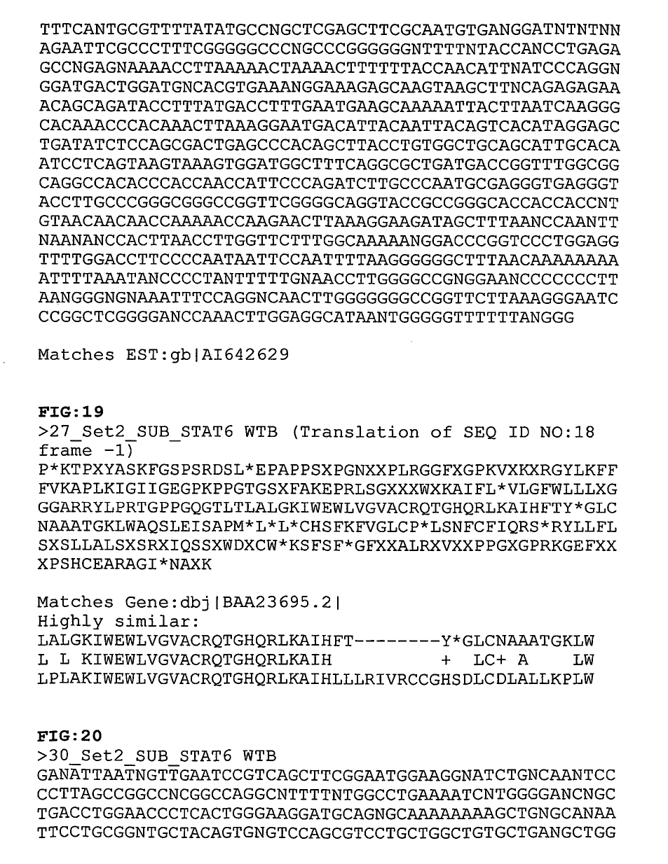

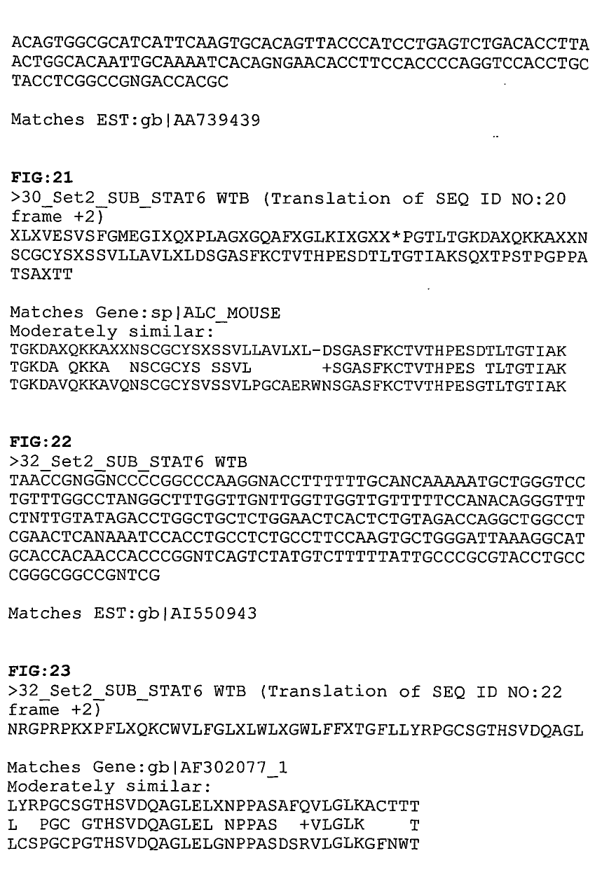

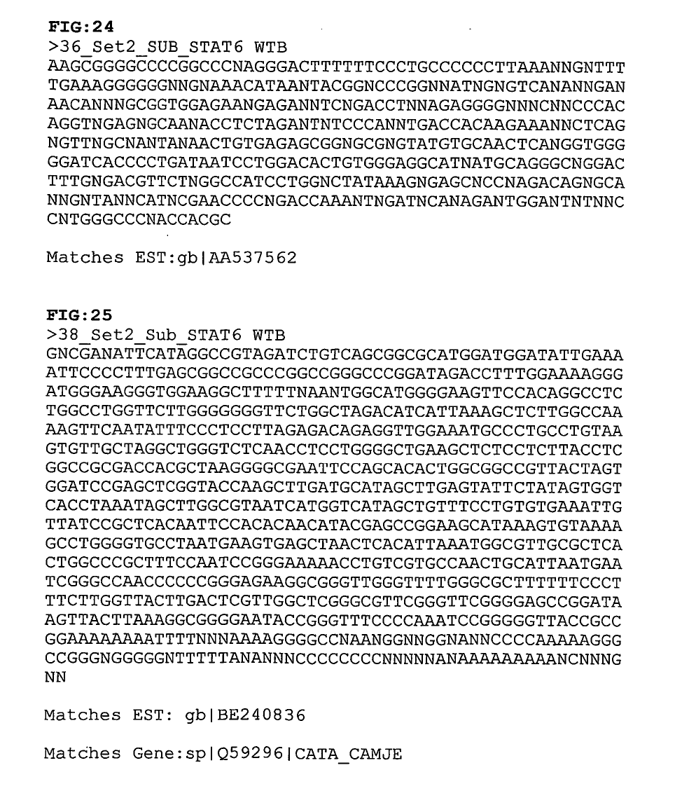

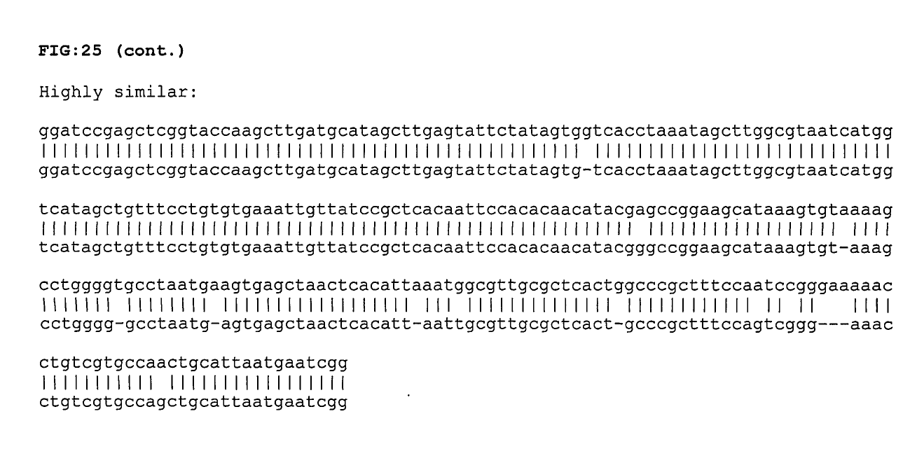

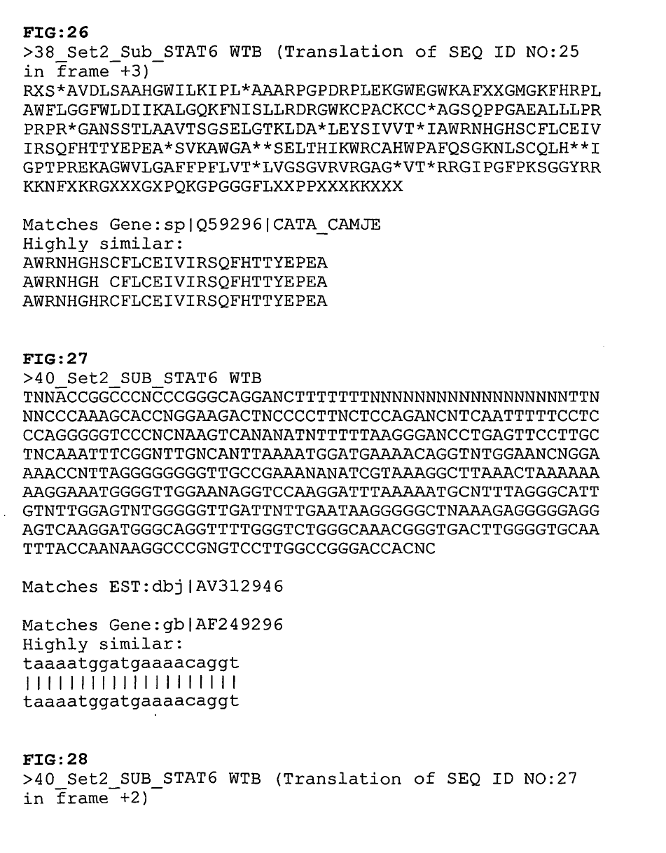

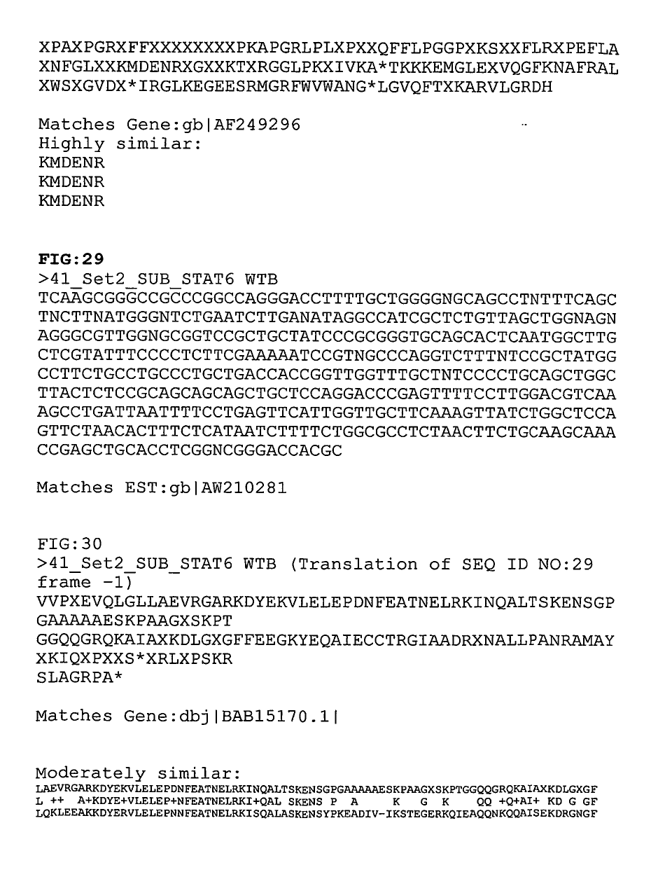

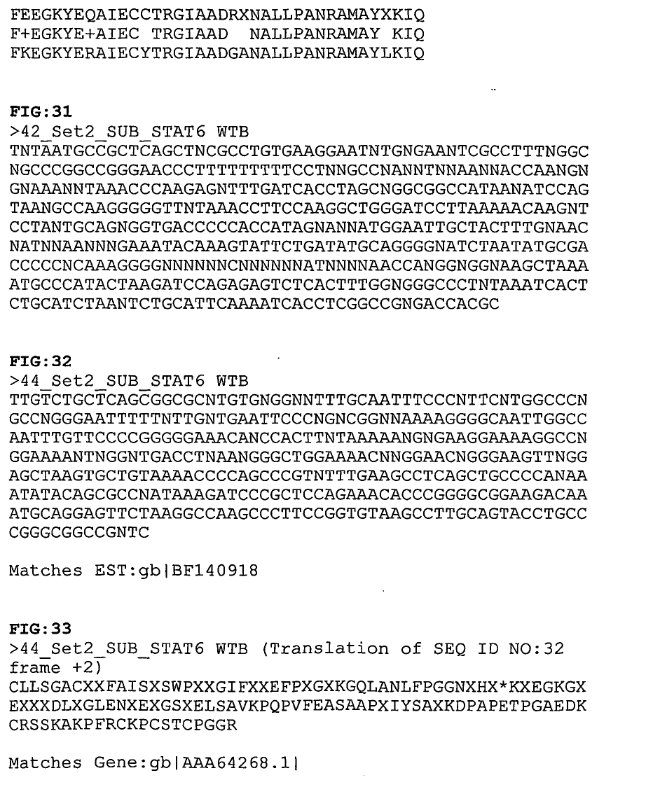

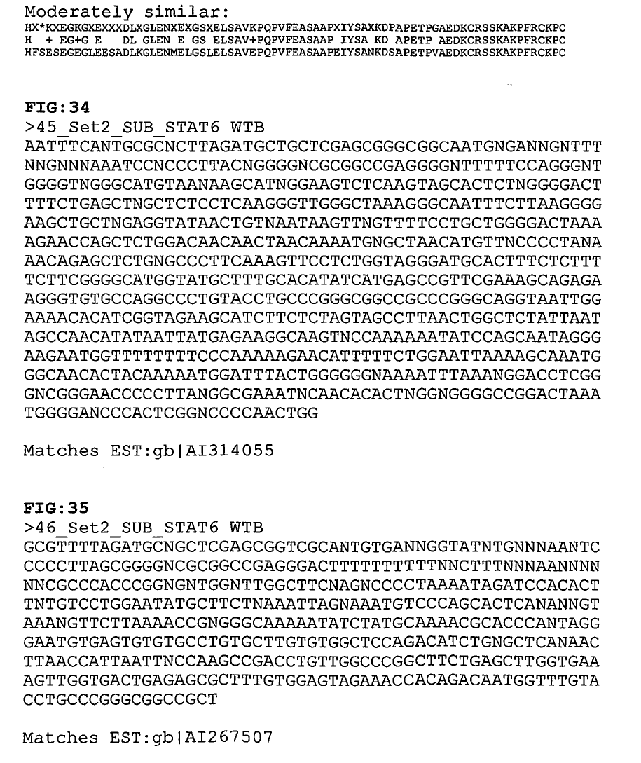

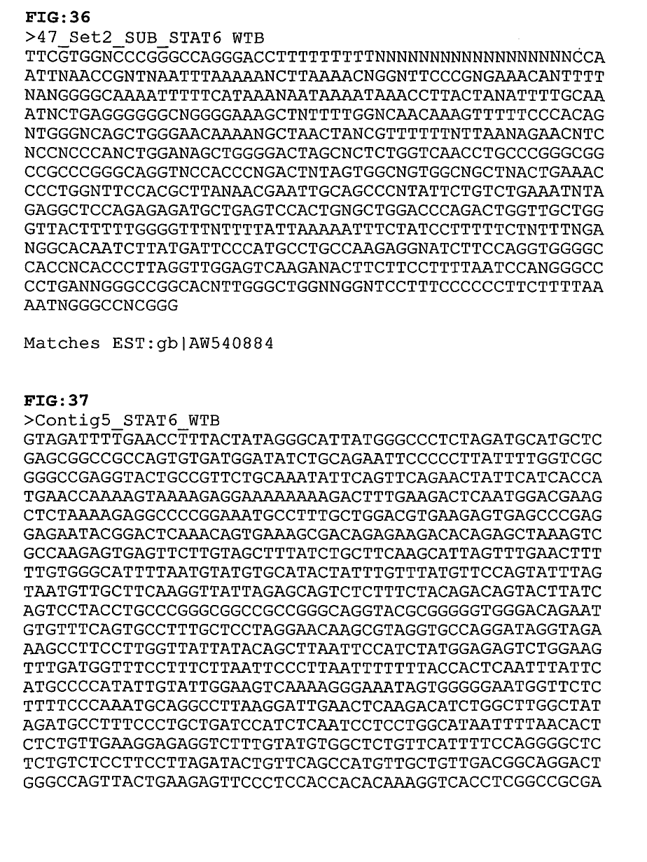

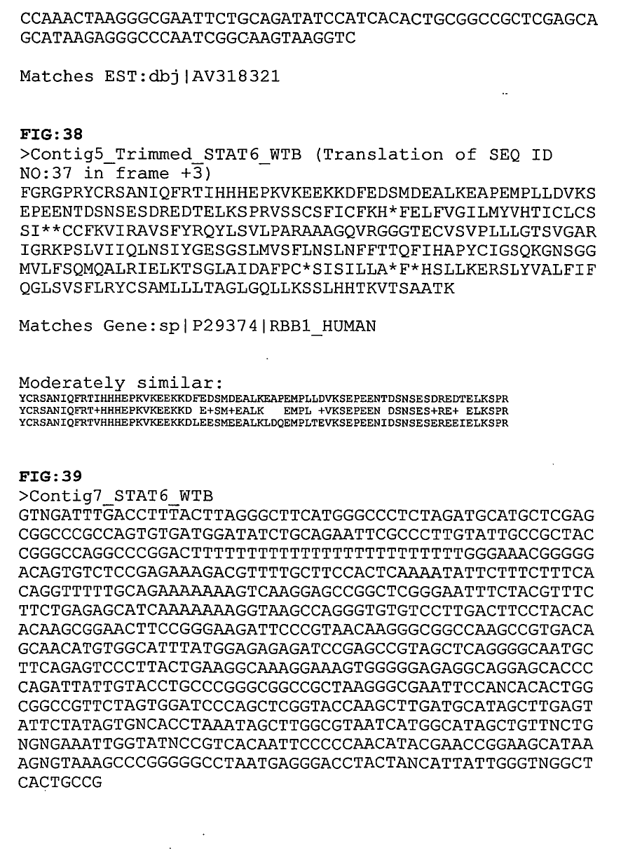

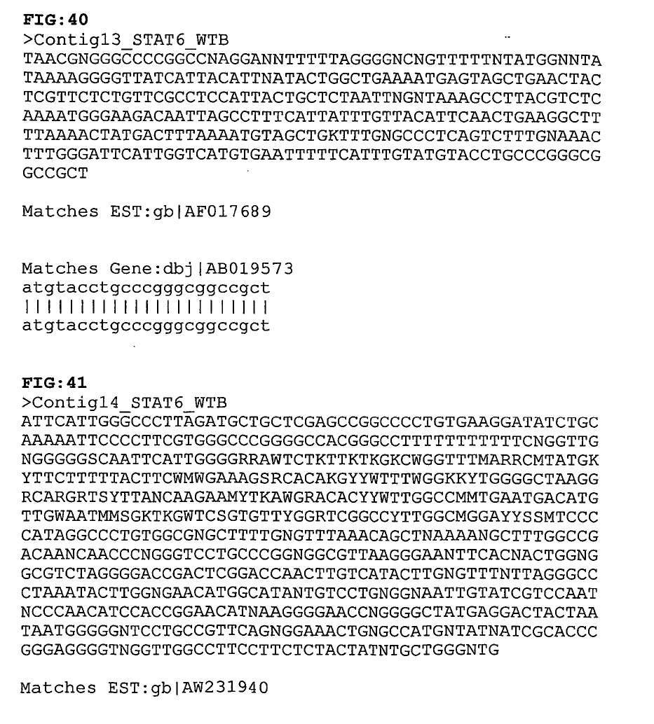

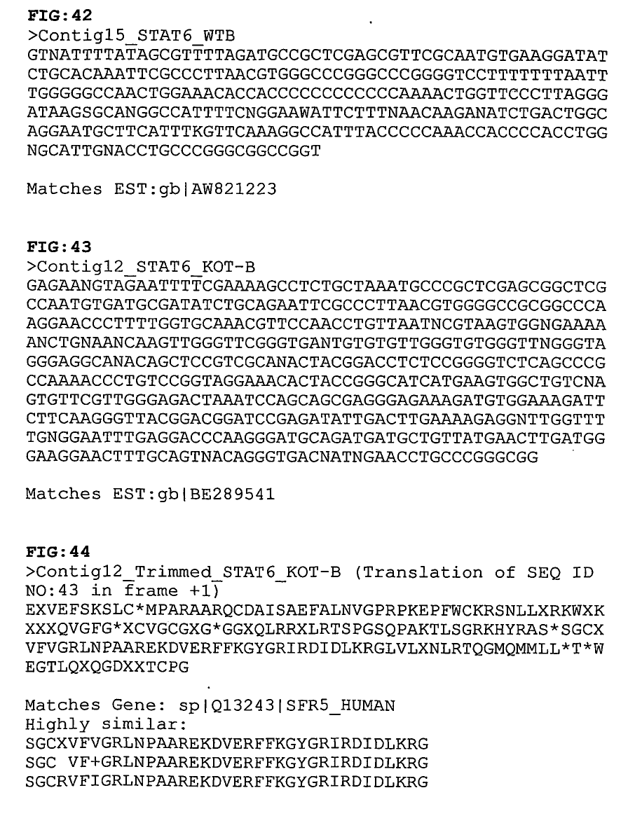

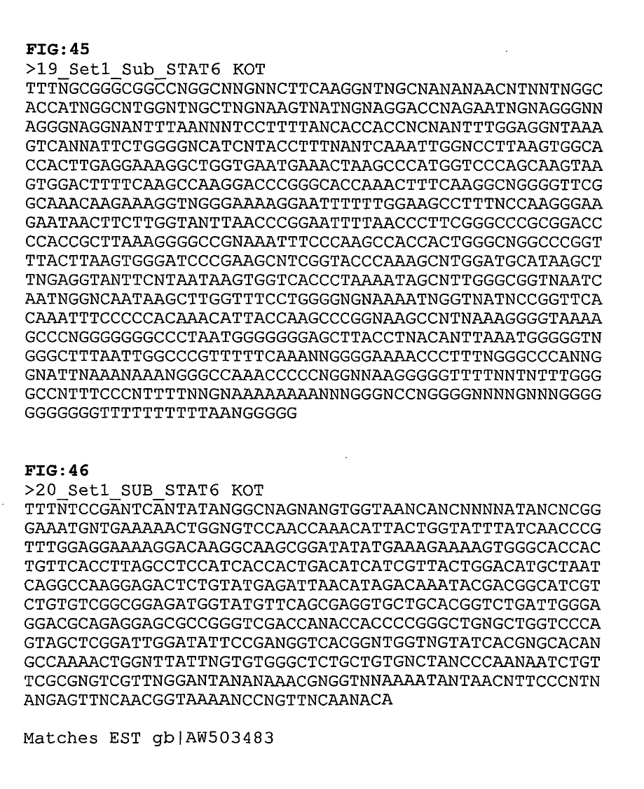

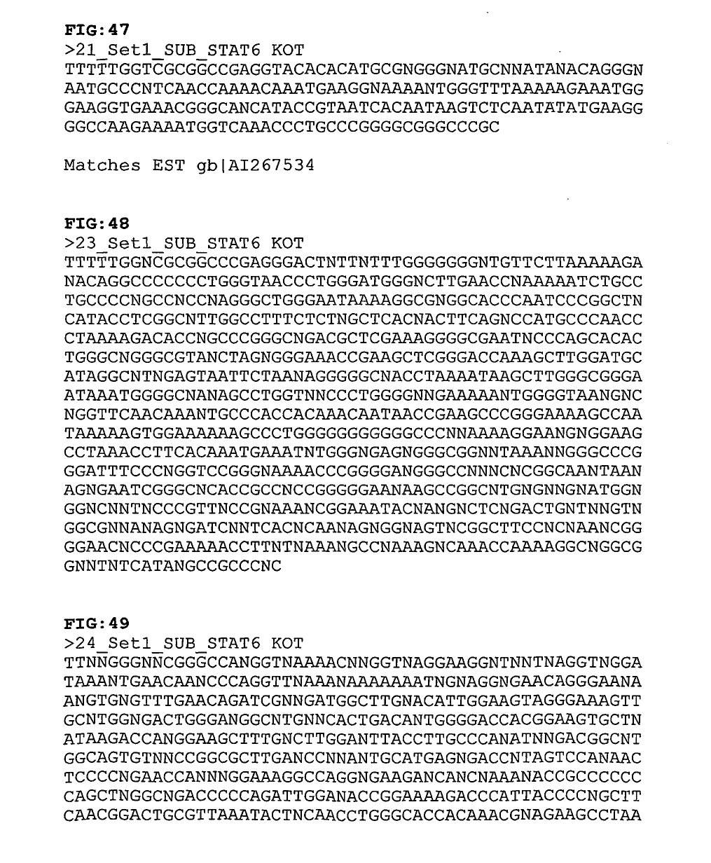

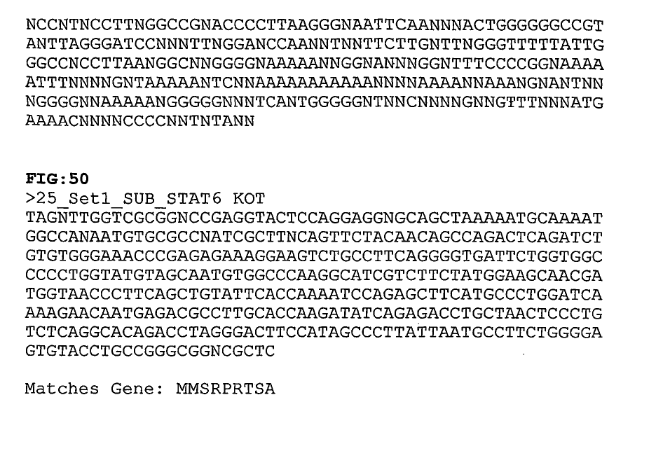

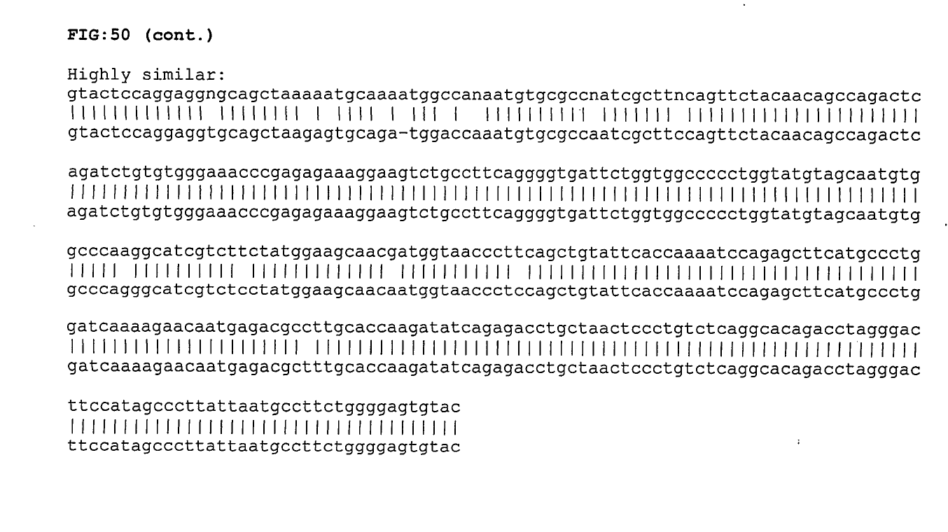

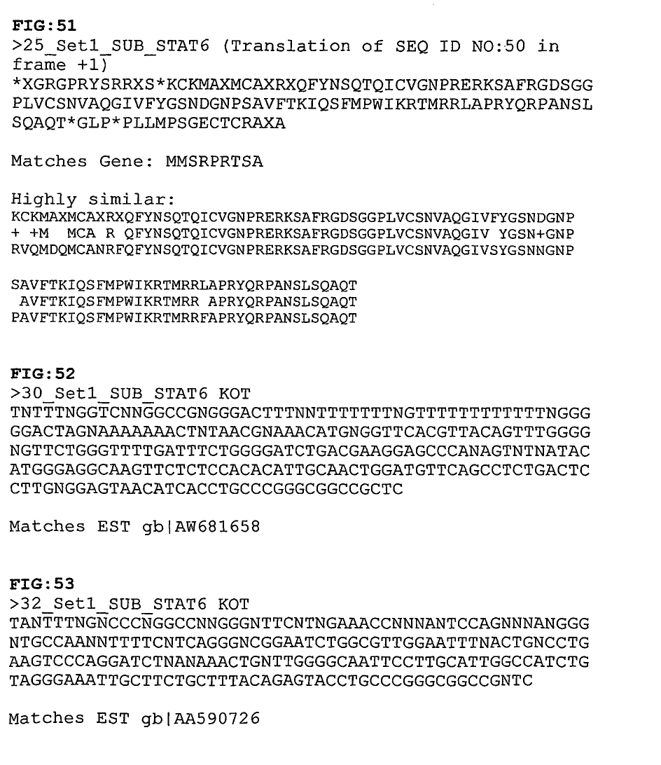

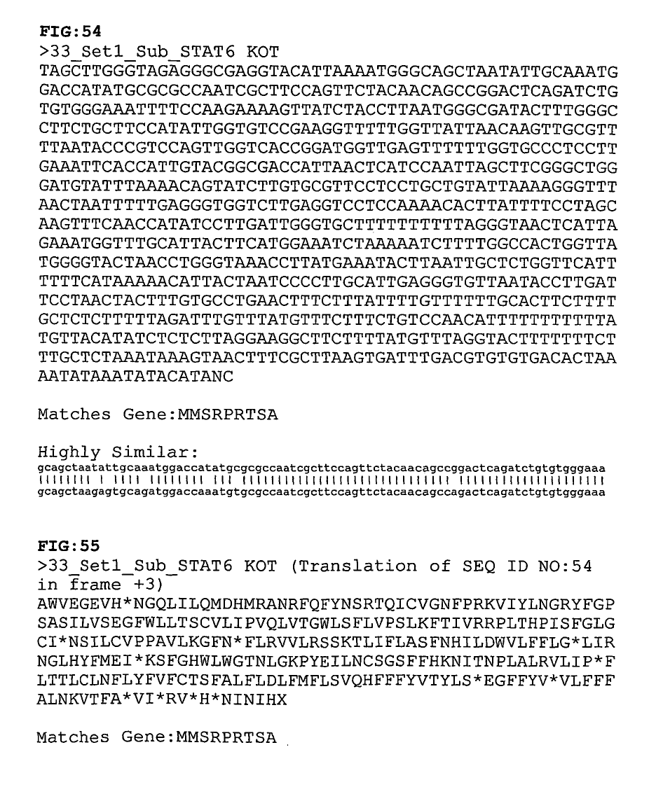

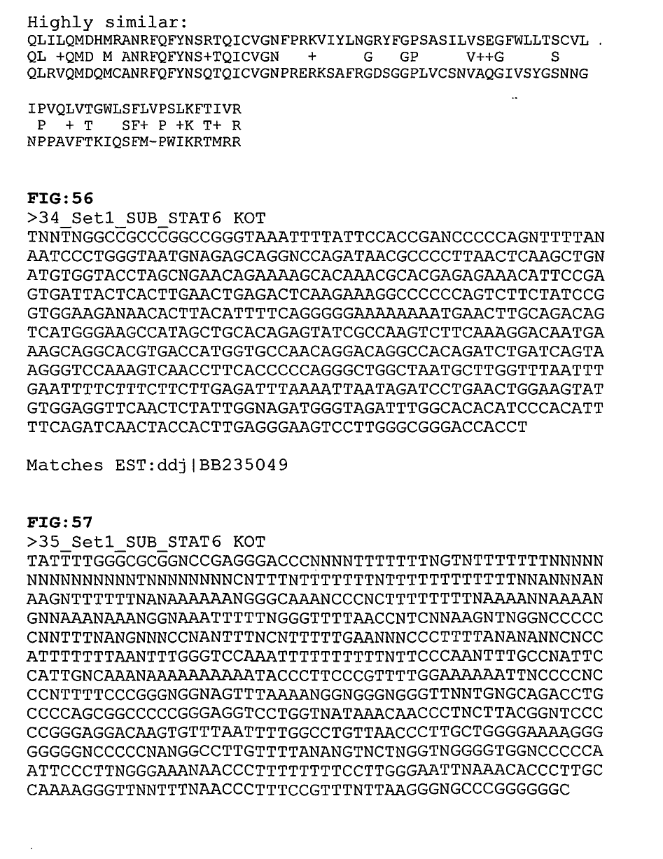

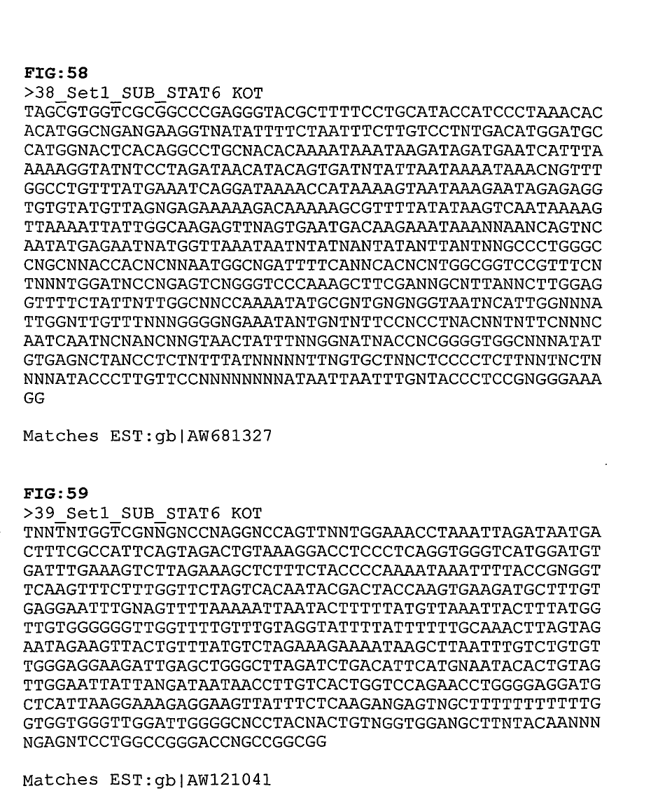

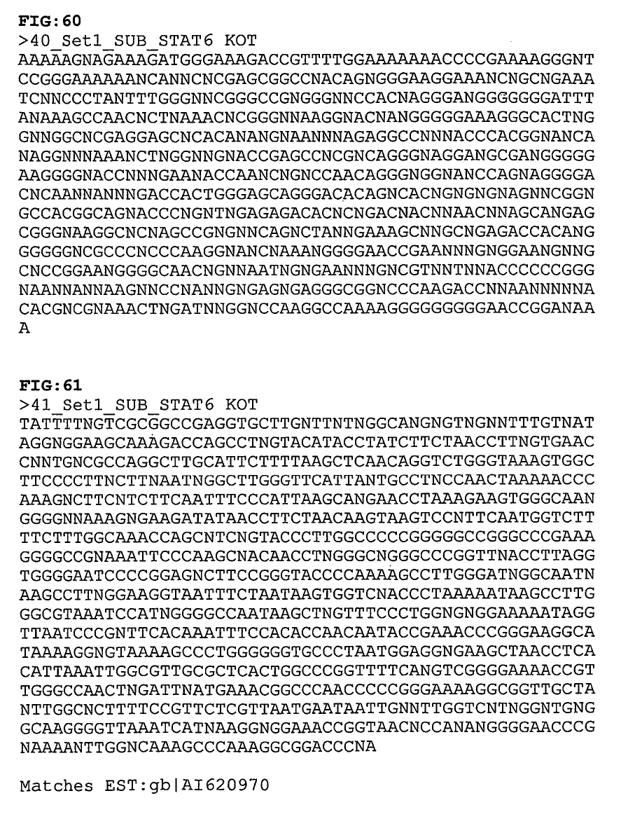

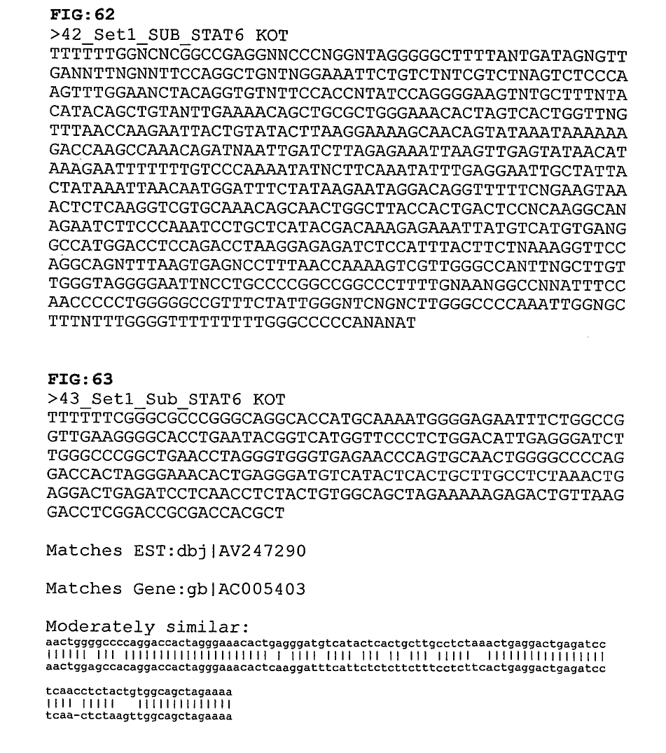

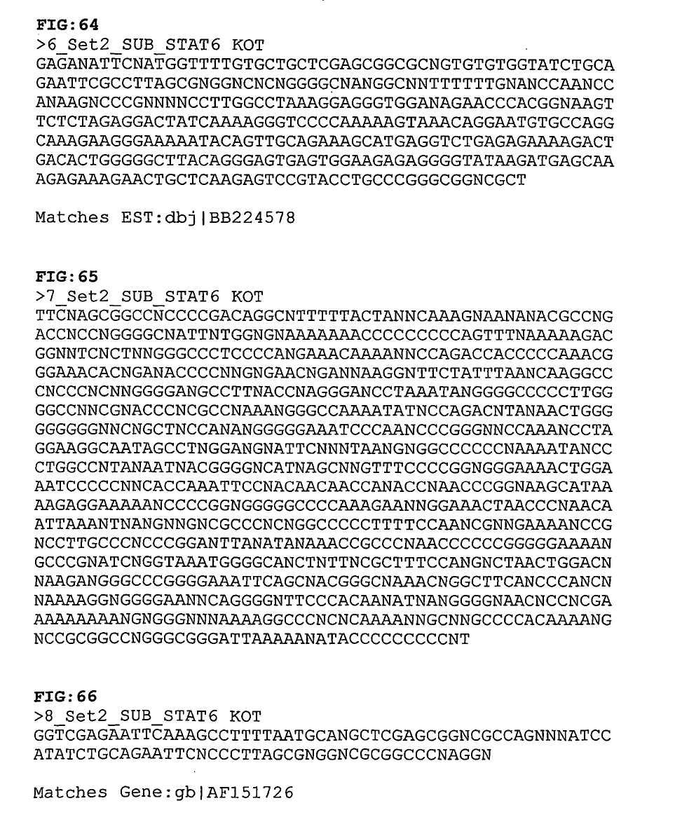

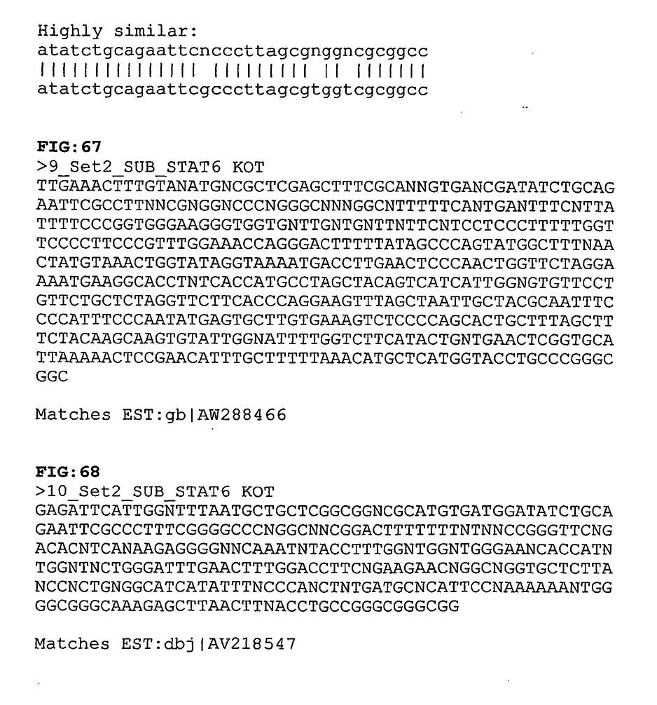

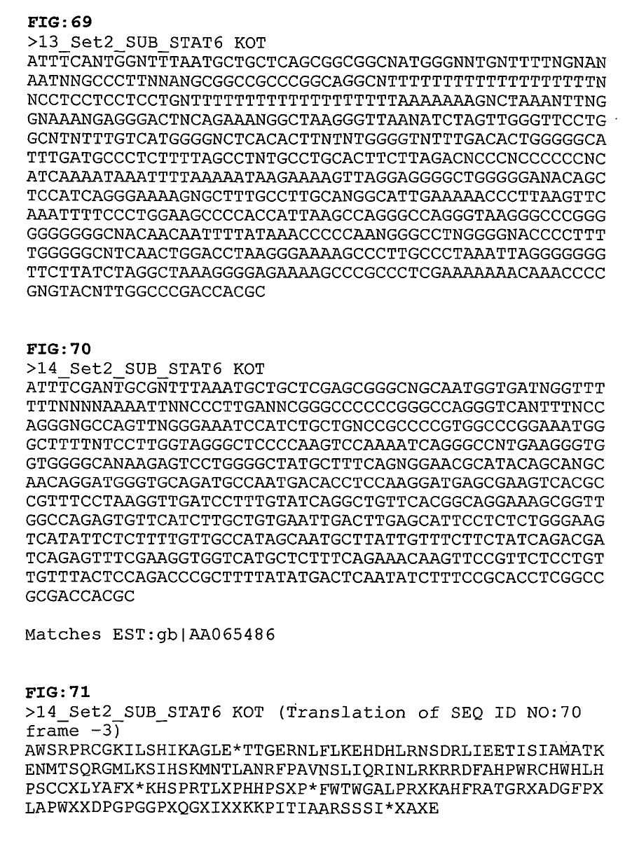

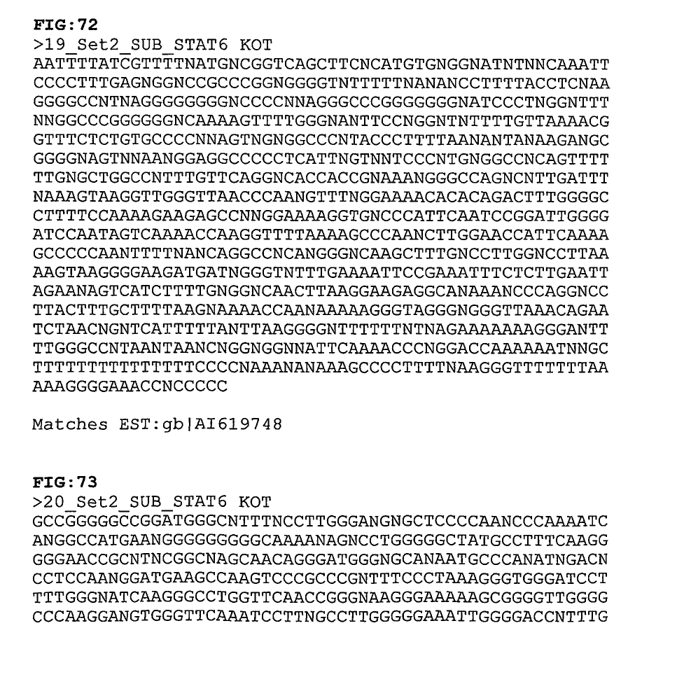

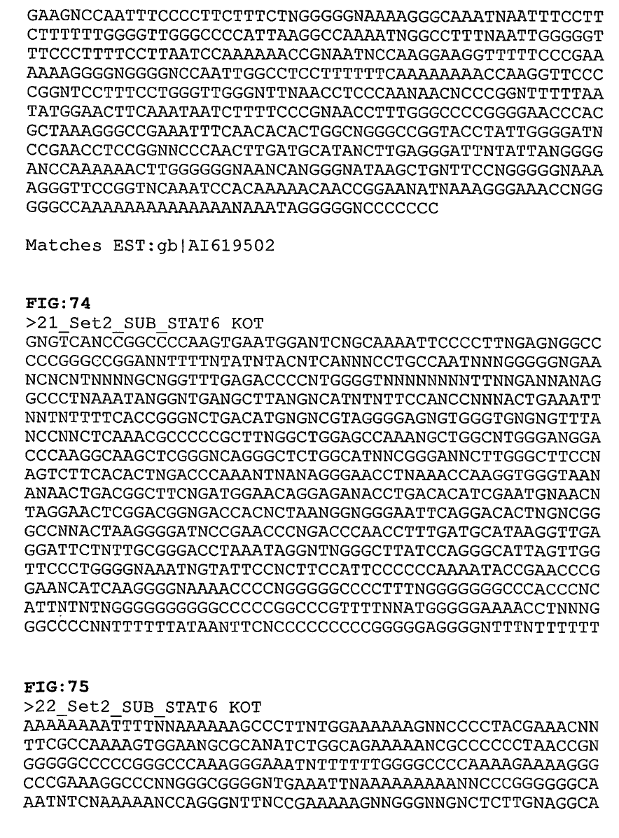

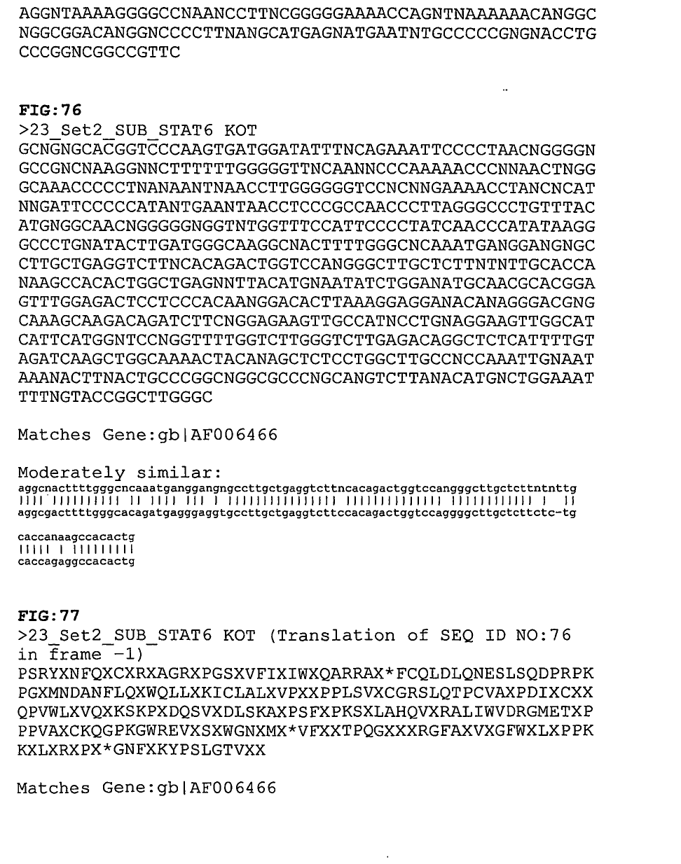

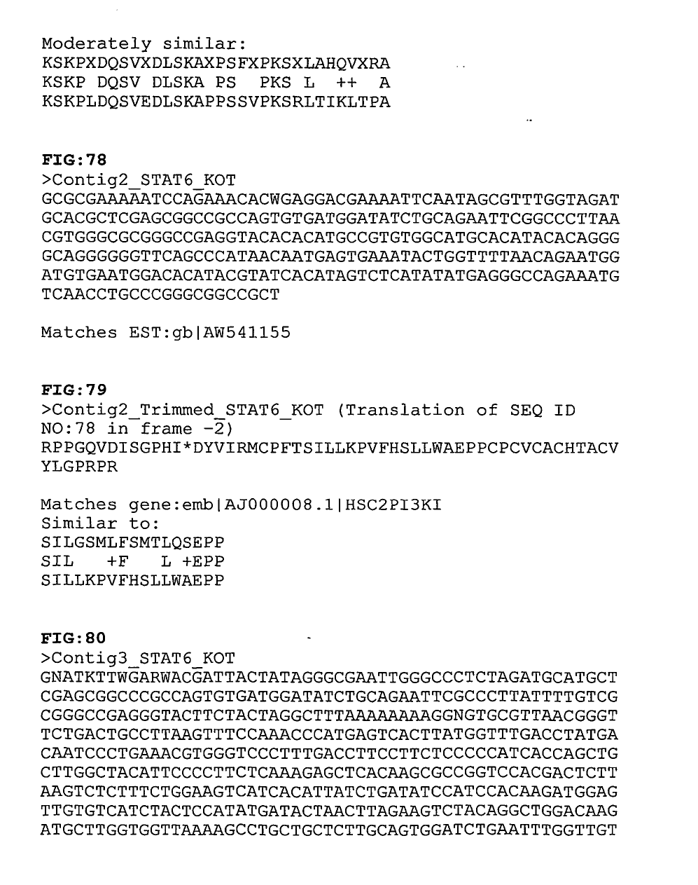

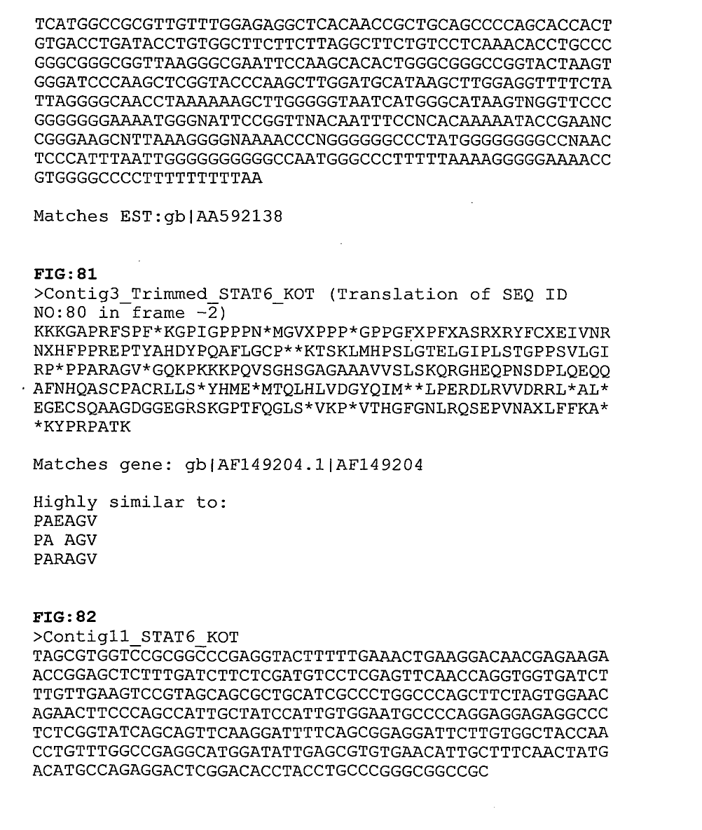

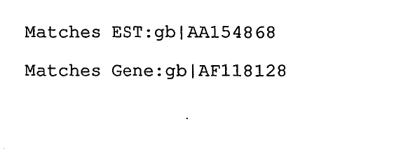

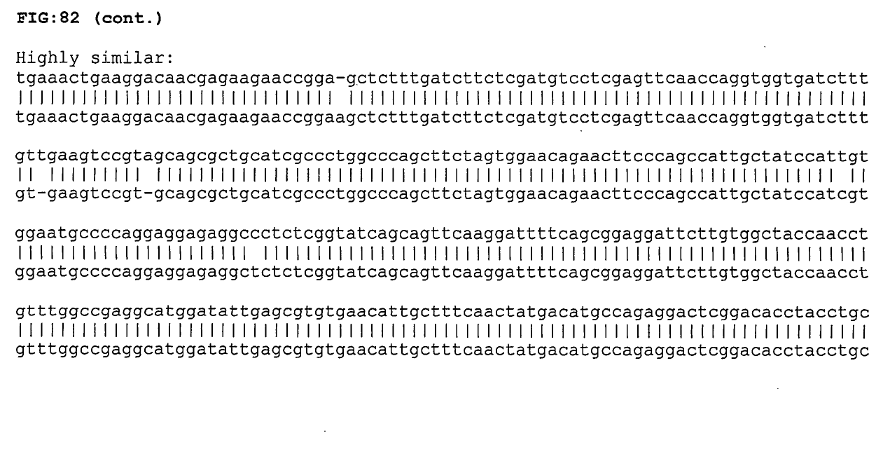

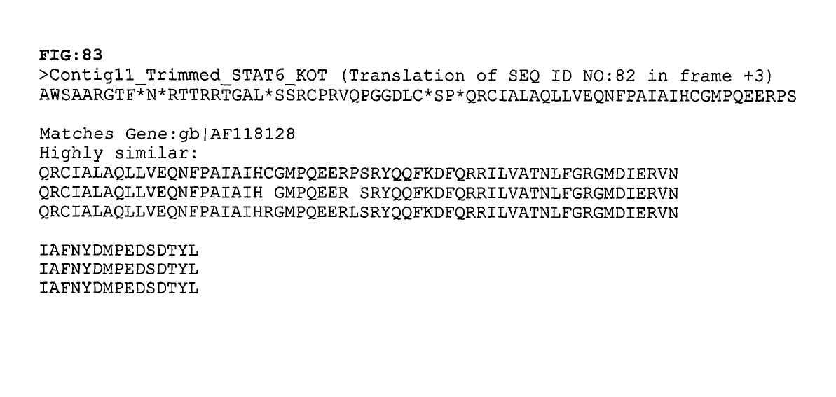

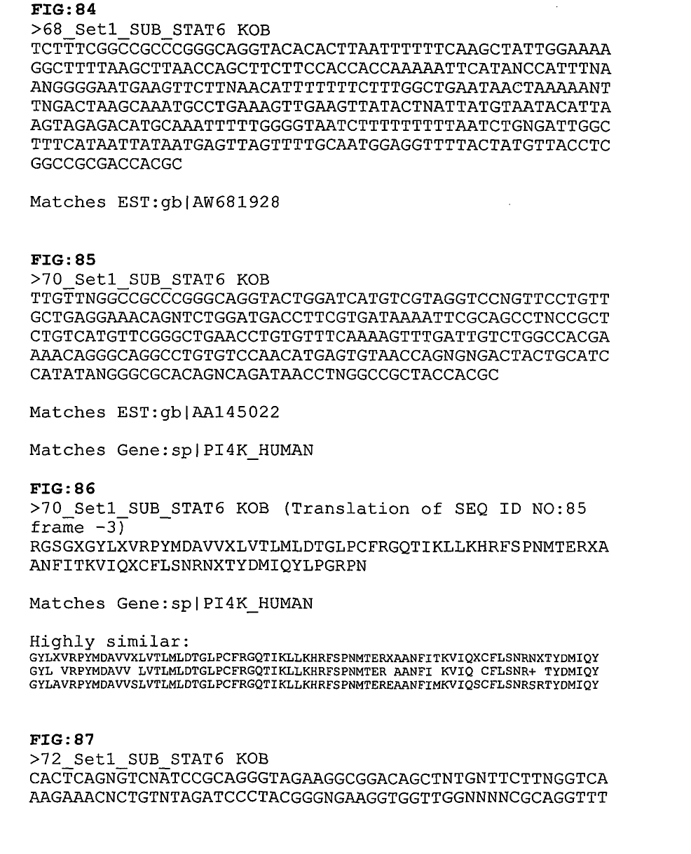

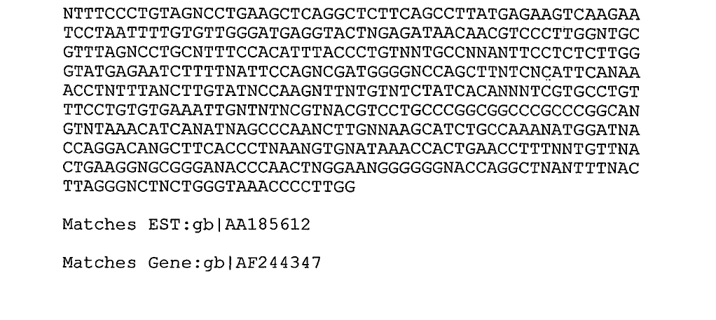

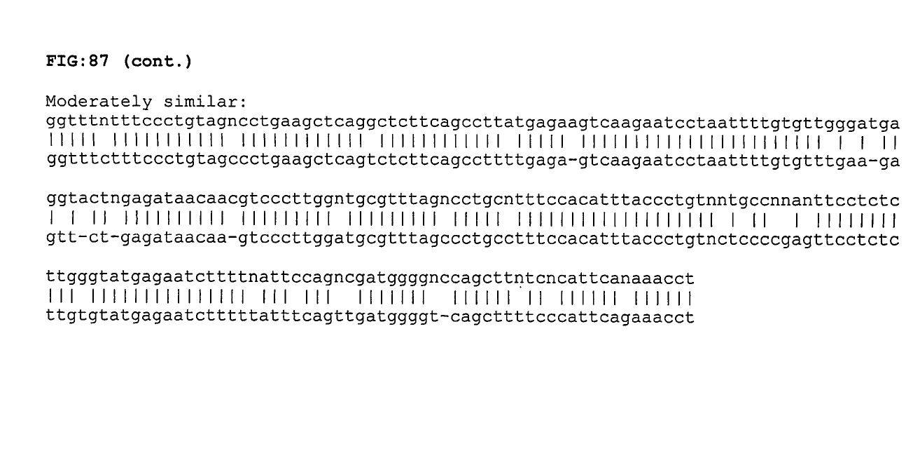

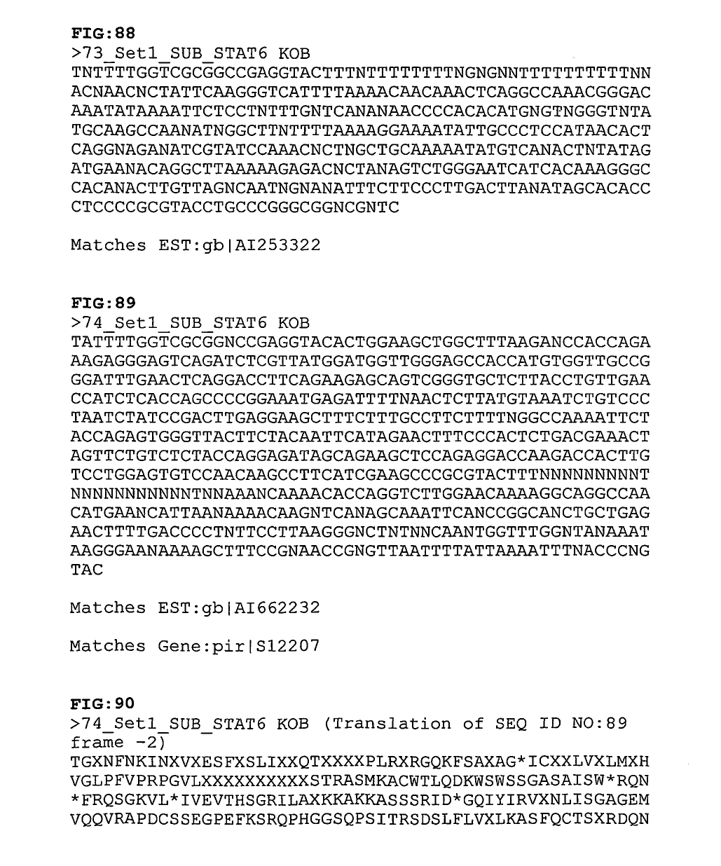

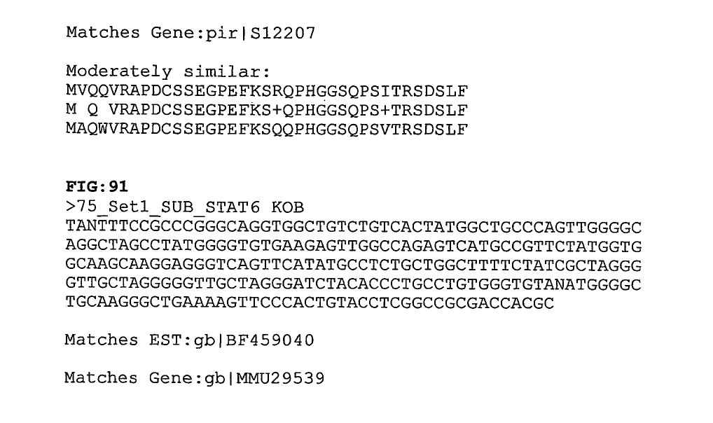

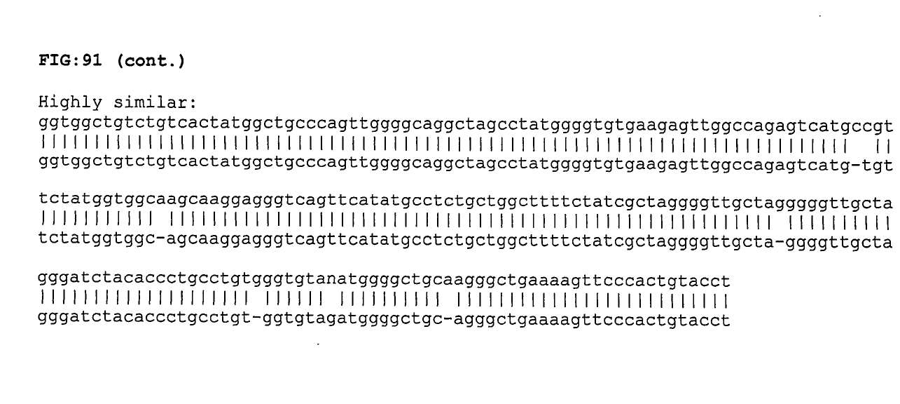

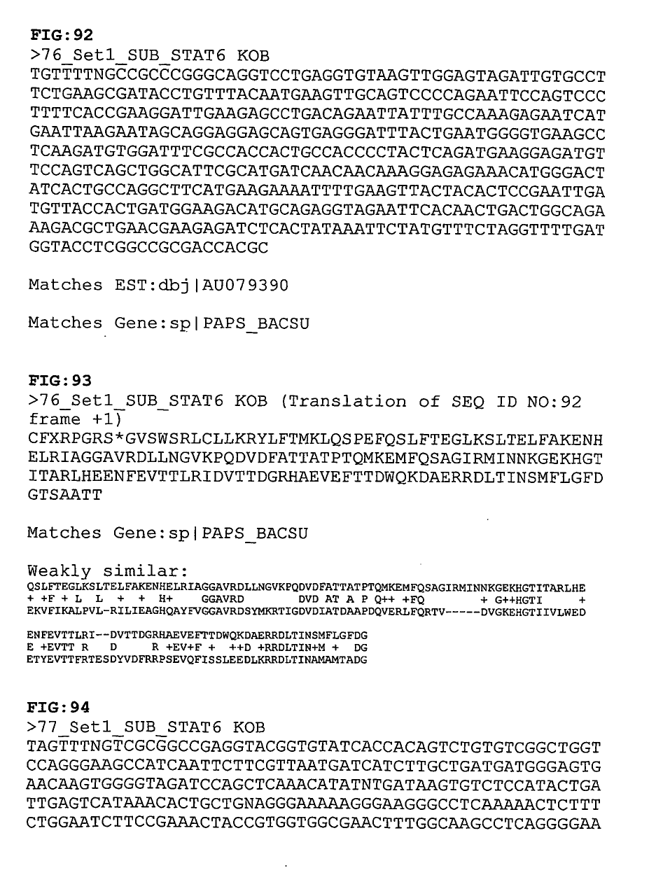

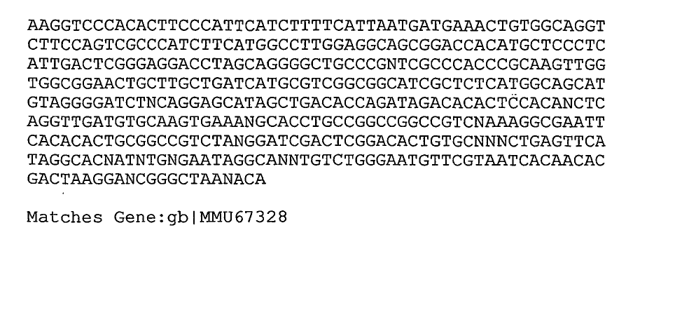

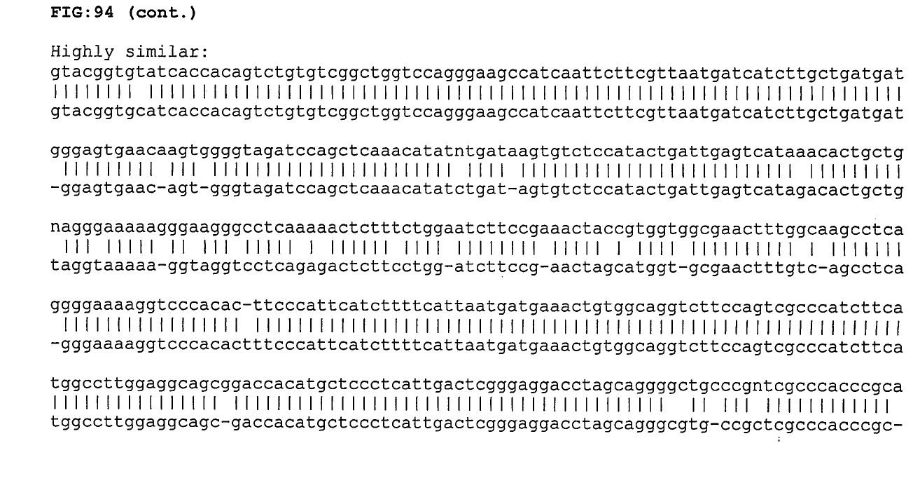

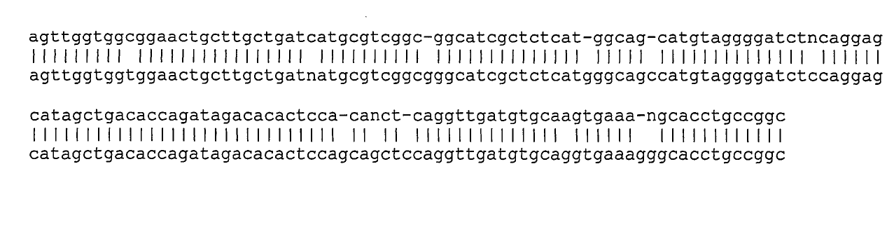

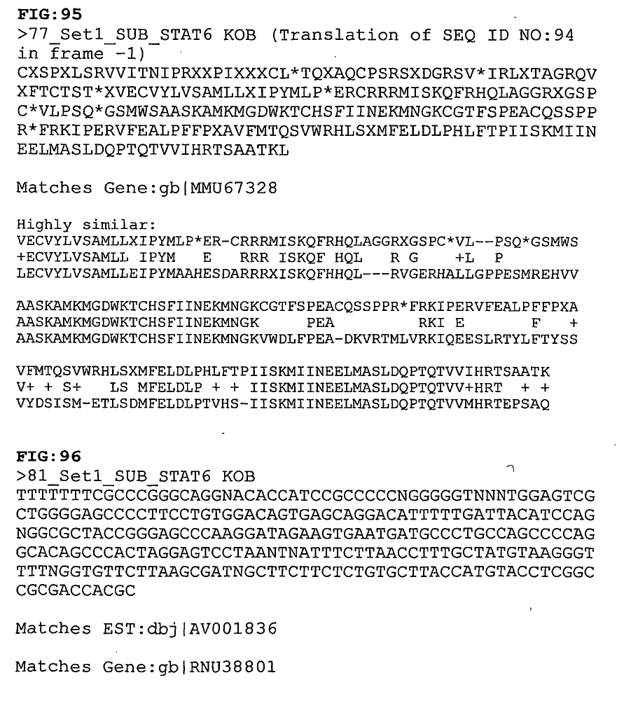

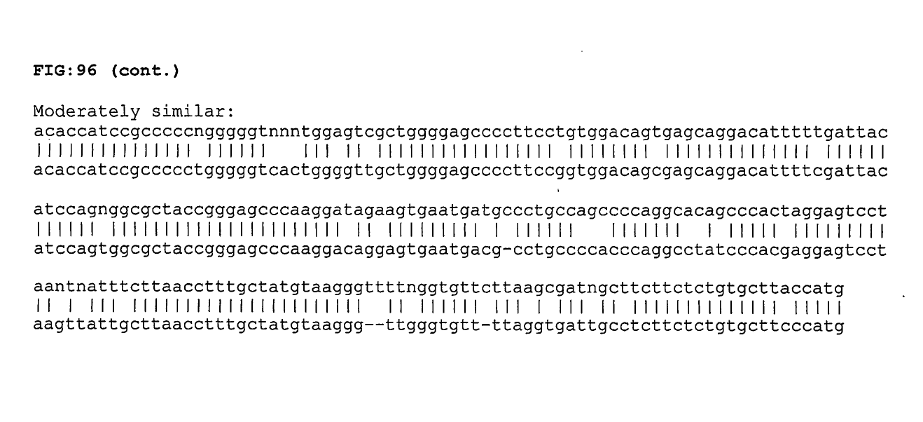

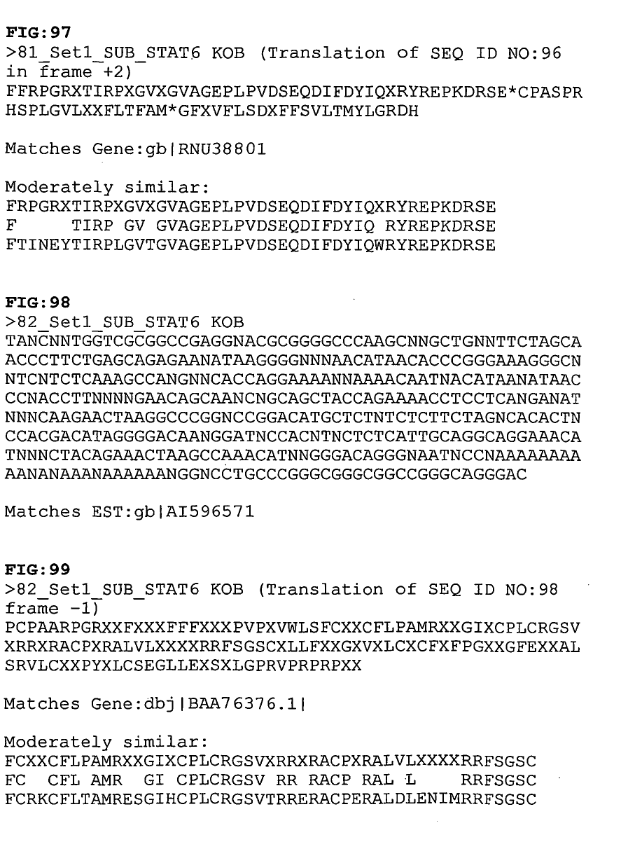

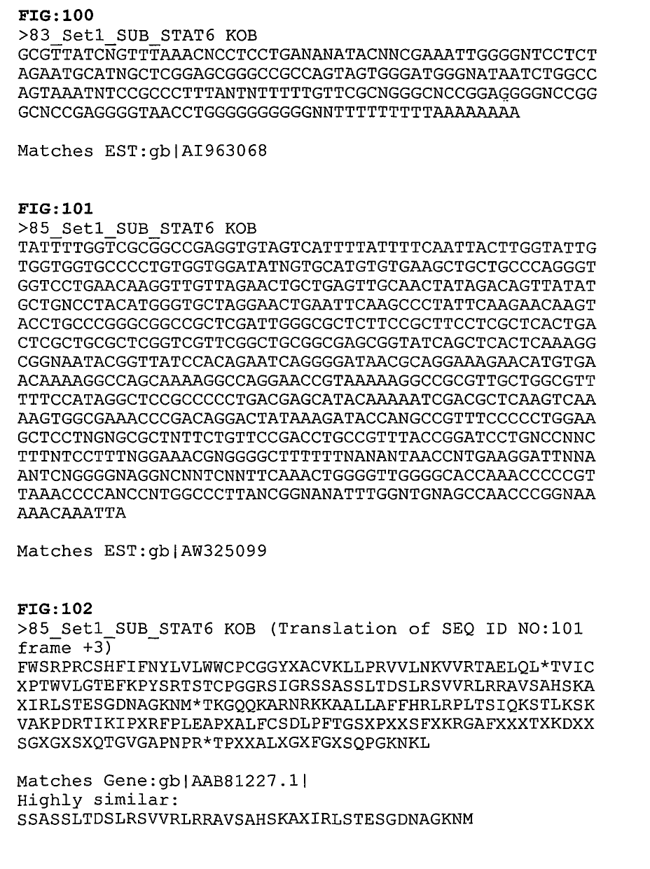

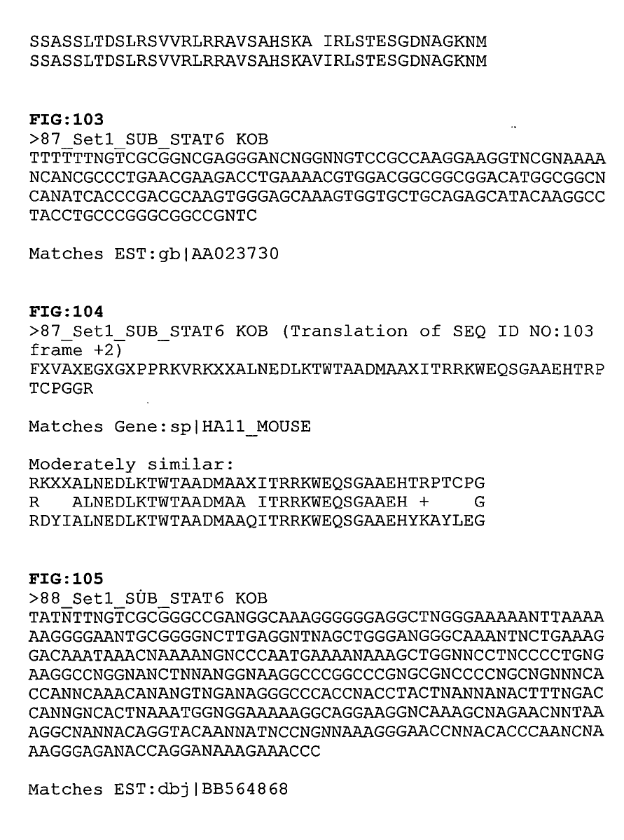

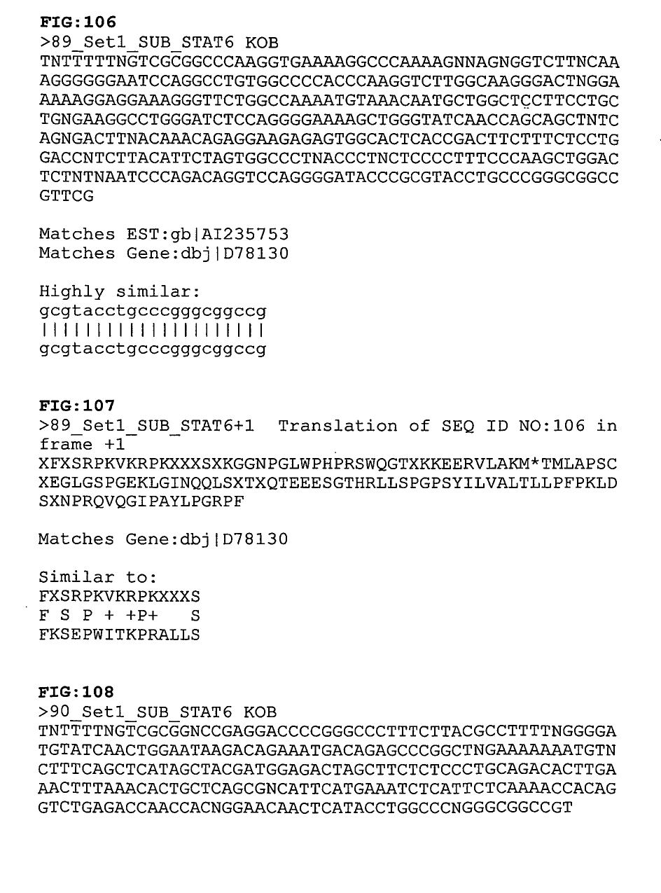

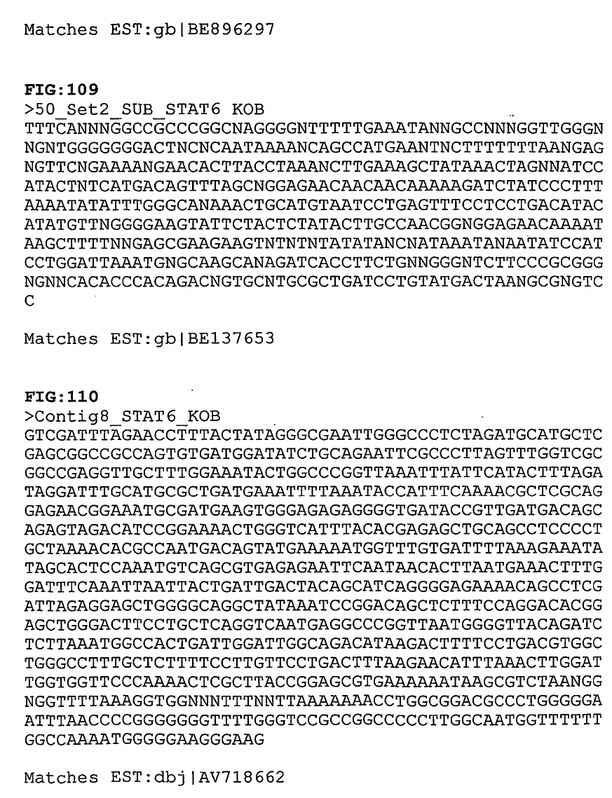

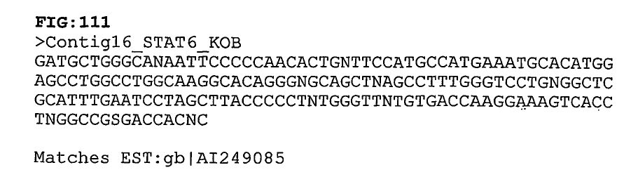

### DETAILED DESCRIPTION OF THE INVENTION

The present invention provides for purified partial immune-related protein cDNAs which were specifically expressed in Stat6^{-/-} T cells, Stat6^{-/-} B cells, wildtype T cells, or wildtype B cells. Stat6^{-/-} knockout mouse was used to identify genes whose transcription can be activated by IL-4 signaling because IL-4 plays a major role in many immune disorders, such as allergy, atopy, and asthma.

Four subtractions can be carried out to remove mutually expressed transcripts and enrich for those transcripts expressed uniquely in each of the four cell populations. In the two subtractions using wildtype mRNA as the tester and Stat6−/− mRNA as the driver, it is expected that the majority of enriched transcripts will be from genes activated by stimulation of the wildtype T and B cells by IL-4. These genes will therefore be important targets for regulation in the treatment of IL-4 mediated disorders. On the other hand, in the two subtractions using Stat6−/− mRNA as the tester and wildtype mRNA as the driver, it is expected that the majority of enriched transcripts will be from genes either normally activated in Stat6−/− cells as a compensatory mechanism for the lack of Stat6, or from normally active genes that are turned off in wildtype T and B cells upon stimulation by IL-4. These genes may therefore be important targets for enhancement in the treatment of IL-4 mediated disorders.

The immune-related polypeptides of the present invention can be used as targets to develop selective inhibitors or activators directed against each of the polypeptide to regulate immune-related disorders.

Polypeptides

Immune-related polypeptides according to the present invention comprise the amino acid sequence shown in any of SEQ ID NO:5, 19, 21, 23, 26, 28, 30, 33, 38, 44, 51, 55, 71, 77, 79, 81, 83, 86, 90, 93, 95, 97, 99, 102, 104, and 107, a portion of that sequence, or a biologically active variant of that amino acid sequence.

Biologically Active Variants

Preferably, naturally or non-naturally occurring variants for immune-related polypeptides of the present invention have amino acid sequences which are at least about 50, preferably about 75, 90, 96, or 98% identical to the amino acid sequence shown in any of SEQ ID NO:5, 19, 21, 23, 26, 28, 30, 33, 38, 44, 51, 55, 71, 77, 79, 81, 83, 86, 90, 93, 95, 97, 99, 102, 104, and 107. Alternatively, naturally or non-naturally occurring variants for immune-related polypeptides of the present invention have amino acid sequences which are at least about 50, preferably about 75, 90, 96, or 98% identical to the amino acid sequence encoded by any of SEQ ID NOs:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111. Percent identity between a putative immune-related variant and an amino acid sequence of SEQ ID NO: 5, 19, 21, 23, 26, 28, 30, 33, 38, 44, 51, 55, 71, 77, 79, 81, 83, 86, 90, 93, 95, 97, 99, 102, 104, and 107 or amino acid sequence encoded by any of SEQ ID NO: 1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 is determined by conventional methods. See, for example, Altschul et al., Bull. Math. Bio. 48:603 (1986), and Henikoff & Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff & Henikoff, 1992.

Those skilled in the art appreciate that there are many established algorithms available to align two amino acid sequences. The “FASTA” similarity search algorithm of Pearson & Lipman is a suitable protein alignment method for examining the level of identity shared by an amino acid sequence disclosed herein and the amino acid sequence of a putative variant. The FASTA algorithm is described by Pearson & Lipman, Proc. Nat'l Acad. Sci. USA 85:2444(1988), and by Pearson, Meth. Enzymol. 183:63 (1990). Briefly, FASTA first characterizes sequence similarity by identifying regions shared by the query sequence (e.g., SEQ ID NO: 2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman & Wunsch, J. Mol. Biol.48:444 (1970); Sellers, SIAM J. Appl. Math.26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of an immune-related polypeptide can be found using computer programs well known in the art, such as DNASTAR software. Whether an amino acid change results in a biologically active Immune-related variant polypeptide can readily be determined by assaying for immune-related polypeptide activity, as described, for example, in the specific examples, below.

Fusion Proteins

Fusion proteins can comprise at least 5, 6, 8, 10, 25, or 50 or more contiguous amino acids of an amino acid sequence shown in any of SEQ ID NOs: 5, 19, 21, 23, 26, 28, 30, 33, 38, 44, 51, 55, 71, 77, 79, 81, 83, 86, 90, 93, 95, 97, 99, 102, 104, and 107; or amino acid sequence encoded by any of SEQ ID NOs:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111. Fusion proteins are useful for generating antibodies against immune-related polypeptide amino acid sequences and for use in various assay systems. For example, fusion proteins can be used to identify proteins which interact with portions of an immune-related polypeptide. Protein affinity chromatography or library-based assays for protein-protein interactions, such as the yeast two-hybrid or phage display systems, can be used for this purpose. Such methods are well known in the art and also can be used as drug screens.

An immune-related polypeptide fusion protein comprises two polypeptide segments fused together by means of a peptide bond. The first polypeptide segment comprises at least 5, 6, 8, 10, 25, or 50 or more contiguous amino acid sequence shown in any of SEQ ID NO:5, 19, 21, 23, 26, 28, 30, 33, 38, 44, 51, 55, 71, 77, 79, 81, 83, 86, 90, 93, 95, 97, 99, 102, 104, and 107 or amino acid sequence encoded by any of SEQ ID NO:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 of a biologically active variant, such as those described above. The first polypeptide segment also can comprise full-length Immune-related polypeptide.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the immune-related polypeptide-encoding sequence and the heterologous protein sequence, so that the immune-related polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the group consisting of SEQ ID NOs:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of the immune-related polypeptide can be obtained using polynucleotides of immune-related gene (described below) to make suitable probes or primers for screening cDNA expression libraries from other species, such as mice, monkeys, or yeast, identifying cDNAs which encode homologs of the immune-related polypeptide, and expressing the cDNAs as is known in the art.

Immune-related Polynucleotides

The polynucleotides of the present invention can be single- or double-stranded and comprise a coding sequence or the complement of a coding sequence for an immune-related polypeptide. The coding sequence for human immune-related polypeptide is shown in SEQ ID NO:1-4, 6-18, 20, 22, 24, 25,27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111.

Degenerate nucleotide sequences encoding human immune-related polypeptides, as well as homologous nucleotide sequences which are at least about 50, preferably about 75, 90, 96, or 98% identical to the nucleotide sequence shown in SEQ ID NOs: 1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 also are immune-related polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of immune-related polynucleotides which encode biologically active immune-related polypeptides also are immune-related polynucleotides.

Identification of Variants and Homologs of Immune-related Polynucleotides

Variants and homologs of the immune-related polynucleotides described above also are immune-related polynucleotides. Typically, homologous immune-related poly-nucleotide sequences can be identified by hybridization of candidate polynucleotides to known immune-related polynucleotides under stringent conditions, as is known in the art. For example, using the following wash conditions-2×SSC (0.3 M NaCl, 0.03 M sodium citrate, pH 7.0), 0.1 % SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the immune-related polynucleotides disclosed herein also can be identified by making suitable probes or primers and screening cDNA expression libraries from other species, such as mice, monkeys, or yeast. Human variants of immune-related polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human immune-related polynucleotides or immune-related polynucleotides of other species can therefore be identified by hybridizing a putative homologous immune-related polynucleotide with a polynucleotide having a nucleotide sequence of any of SEQ ID NO:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94; 96, 98, 100, 101, 103, 105, 106, 108, 109, 110, and 111 or the complement thereof to form a test hybrid. The melting temperature of the test hybrid is compared with the melting temperature of a hybrid comprising trans-formylase polynucleotides having perfectly complementary nucleotide sequences, and the number or percent of basepair mismatches within the test hybrid is calculated.

Nucleotide sequences which hybridize to the polynucleotides of the present invention or their complements following stringent hybridization and/or wash conditions also are immune-related polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between an immune-related polynucleotide having a nucleotide sequence shown in any of SEQ ID NOs:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. USA 48, 1390 (1962):
Tₘ=81.5° C.−16.6(log₁₀[Na⁺])+0.41(%G+C)−0.63(% formamide)−600/l),
where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Immune-related Polynucleotides

A naturally occurring immune-related polynucleotides can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated immune-related polynucleotides. For example, restriction enzymes and probes can be used to isolate polynucleotide fragments which comprise immune-related nucleotide sequences. Isolated polynucleotides are in preparations which are free or at least 70, 80, or 90% free of other molecules.

Immune-related cDNA molecules can be made with standard molecular biology techniques, using immune-related mRNA as a template. Immune-related cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes immune-related polynucleotides. The degeneracy of the genetic code allows alternate nucleotide sequences to be synthesized which will encode an immune-related polypeptide having, for example, an amino acid sequence shown in any of SEQ ID NOs: 5, 19, 21, 23, 26, 28, 30, 33, 38, 44, 51, 55, 71, 77, 79, 81, 83, 86, 90, 93, 95, 97, 99, 102, 104, and 107 or a biologically active variant thereof.

Extending Immune-related Polynucleotides

Various PCR-based methods can be used to extend the nucleic acid sequences encoding the disclosed portions of human immune-related polypeptide to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72 ° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Immune-related Polypeptides

Immune-related polypeptides can be obtained, for example, by purification from human cells, by expression of immune-related polynucleotides, or by direct chemical synthesis.

Protein Purification

Immune-related polypeptides can be purified from any human cell that expresses the protein, including host cells that have been transfected with immune-related polynucleotides. Thymus, spleen, lymph node, and other immune-related tissues are particularly useful sources of the polypeptides of the present invention. A purified immune-related polypeptide is separated from other compounds which normally associate with the immune-related polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

A preparation of purified immune-related polypeptides is at least 80% pure; preferably, the preparations are 90%, 95%, or 99% pure. Purity of the preparations can be assessed by any means known in the art, such as SDS-polyacrylamide gel electrophoresis.

Expression of Immune-related Polynucleotides

To express an immune-related polypeptide of the present invention, an immune-related polynucleotide can be inserted into an expression vector which contains the necessary elements for the transcription and translation of the inserted coding sequence. Methods which are well known to those skilled in the art can be used to construct expression vectors containing sequences encoding immune-related polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding an immune-related polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding an immune-related polypeptide, vectors based on SV40 or EBV can be used with an appropriate selectable marker.

Bacterial and Yeast Expression Systems

In bacterial systems, a number of expression vectors can be selected depending upon the use intended for the immune-related polypeptide. For example, when a large quantity of an immune-related polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the immune-related polypeptide can be ligated into the vector in frame with sequences for the amino-terminal Met and the subsequent 7 residues of β-galactosidase so that a hybrid protein is produced. pIN vectors (Van Heeke & Schuster, J Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding immune-related polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196, 1992).

An insect system also can be used to express an immune-related polypeptide. For example, in one such system Autographa californica nuclear polyhedrosis virus (AcNPV) is used as a vector to express foreign genes in Spodoptera frugiperda cells or in Trichoplusia larvae. Sequences encoding immune-related polypeptides of the present invention can be cloned into a non-essential region of the virus, such as the polyhedrin gene, and placed under control of the polyhedrin promoter. Successful insertion of immune-related polypeptides will render the polyhedrin gene inactive and produce recombinant virus lacking coat protein. The recombinant viruses can then be used to infect S. frugiperda cells or Trichoplusia larvae in which immune-related polypeptides can be expressed (Engelhard et al., Proc. Nat. Acad Sci. 91, 3224-3227, 1994).

Mammalian Expression Systems

A number of viral-based expression systems can be used to express immune-related polypeptides in mammalian host cells. For example, if an adenovirus is used as an expression vector, sequences encoding immune-related polypeptides can be ligated into an adenovirus transcription/translation complex comprising the late promoter and tripartite leader sequence. Insertion in a non-essential E1 or E3 region of the viral genome can be used to obtain a viable virus which is capable of expressing an immune-related polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding immune-related polypeptides. Such signals include the ATG initiation codon and adjacent sequences. In cases where sequences encoding an immune-related polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed immune-related polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, B-lymphoma cells and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express immune-related polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced immune-related sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk³¹ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression of Immune-related Polypeptides

Although the presence of marker gene expression suggests that the immune-related polynucleotide is also present, its presence and expression may need to be confirmed. For example, if a sequence encoding an immune-related polypeptide is inserted within a marker gene sequence, transformed cells containing sequences which encode an immune-related polypeptide can be identified by the absence of marker gene function. Alternatively, a marker gene can be placed in tandem with a sequence encoding an immune-related polypeptide under the control of a single promoter. Expression of the marker gene in response to induction or selection usually indicates expression of the immune-related polynucleotide.

Alternatively, host cells which contain an immune-related polynucleotide and which express an immune-related polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding an immune-related polypeptide can be detected by DNA-DNA or DNA-RNA hybridization or amplification using probes or fragments or fragments of polynucleotides encoding an immune-related polypeptide. Nucleic acid amplification-based assays involve the use of oligonucleotides selected from sequences encoding an immune-related polypeptide to detect transformants which contain an immune-related polynucleotide.

A variety of protocols for detecting and measuring the expression of an immune-related polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on an immune-related polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding immune-related polypeptides include oligolabeling, nick translation, end-labeling, or PCR amplification using a labeled nucleotide. Alternatively, sequences encoding an immune-related polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Immune-related Polypeptides

Host cells transformed with nucleotide sequences encoding an immune-related polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode immune-related polypeptides can be designed to contain signal sequences which direct secretion of soluble immune-related polypeptides through a prokaryotic or eukaryotic cell membrane or which direct the membrane insertion of membrane-bound immune-related polypeptide.

As discussed above, other constructions can be used to join a sequence encoding an immune-related polypeptide to a nucleotide sequence encoding a polypeptide domain that will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the immune-related polypeptide also can be used to facilitate purification. One such expression vector provides for expression of a fusion protein containing an immune-related polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the immune-related polypeptide from the fusion protein. Vectors which contain fusion proteins are disclosed in Kroll et al., DNA Cell Biol. 12, 441-453, 1993.

Chemical Synthesis

Sequences encoding an immune-related polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, an immune-related polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of immune-related polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic immune-related polypeptide can be confirmed by amino acid analysis or sequencing (e.g., the Edman degradation procedure; see Creighton, supra). Additionally, any portion of the anion acid sequence of the immune-related polypeptide can be altered during direct synthesis and/or combined using chemical methods with sequences from other proteins to produce a variant polypeptide or a fusion protein.

Production of Altered Immune-related Polypeptides

As will be understood by those of skill in the art, it may be advantageous to produce immune-related polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter immune-related polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of an immune-related polypeptide. “Antibody” as used herein includes intact immunoglobulin molecules, as well as fragments thereof, such as Fab, F(ab′)₂, and Fv, which are capable of binding an epitope of an immune-related polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of an immune-related polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to an immune-related polypeptide provides a detection signal at least 5-, 10-, or 20-fold higher than a detection signal provided with other proteins when used in an inmmunochemical assay. Preferably, antibodies which specifically bind to immune-related polypeptides do not detect other proteins in immunochemical assays and can immunoprecipitate an immune-related polypeptide from solution.

Immune-related polypeptides can be used to immunize a mammal, such as a mouse, rat, rabbit, guinea pig, monkey, or human, to produce polyclonal antibodies. If desired, an immune-related polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g. aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to an immune-related polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to an immune-related polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to immune-related polypeptides. Antibodies with related specificity, but of distinct idiotypic composition, can be generated by chain shuffling from random combinatorial immunoglobin libraries (Burton, Proc. Natl. Acad. Sci. 88, 11120-23,1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to immune-related polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which an immune-related polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of immune-related gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of immune-related gene expression can be obtained by designing antisense oligonucleotides which will form duplexes to the control, 5′, or regulatory regions of the immune-related gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of an immune-related polynucleotide. Antisense oligonucleotides which comprise, for example, 2, 3, 4, or 5 or more stretches of contiguous nucleotides which are precisely complementary to an immune-related polynucleotide, each separated by a stretch of contiguous nucleotides which are not complementary to adjacent immune-related nucleotides, can provide sufficient targeting specificity for immune-related mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular immune-related polynucleotide sequence.

Antisense oligonucleotides can be modified without affecting their ability to hybridize to an immune-related polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of an immune-related polynucleotide can be used to generate ribozymes which will specifically bind to mRNA transcribed from the immune-related polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within an immune-related RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate immune-related RNA targets also can be evaluated by testing accessibility to hybridization with complementary oligonucleotides using ribonuclease protection assays. The nucleotide sequences shown in any of SEQ ID NO:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111 and their complements provide a source of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease immune-related expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Screening Methods

The invention provides assays for screening test compounds which bind to or modulate the activity of an immune-related polypeptide or an immune-related polynucleotide. A test compound preferably binds to an immune-related polypeptide or polynucleotide. More preferably, a test compound decreases or increases the effect of IL-4 as mediated via human immune-related gene or polypeptide by at least about 10, preferably about 50, more preferably about 75, 90, or 100% relative to the absence of the test compound.

Test Compounds

Test compounds can be pharmacological agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead, one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. USA 90, 6909, 1993; Erb et al. Proc. Natl. Acad. Sci. USA 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed Engl. 33, 2061; Gallop et al., J. Med. Chem. 37, 1233, 1994). Libraries of compounds can be presented in solution (see, e.g., Houghten, Biotechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. USA 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to immune-related polypeptides or polynucleotides or to immune-related protein's activity or immune-related gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. USA 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies the active site of the immune-related polypeptide, thereby making the active site inaccessible or accessible to substrate such that normal biological activity is prevented. Examples of such small molecules include, but are not limited to, small peptides or peptide-like molecules.

In binding assays, either the test compound or the immune-related polypeptide can comprise a detectable label, such as a fluorescent, radioisotopic, chemiluminescent, or enzymatic label, such as horseradish peroxidase, alkaline phosphatase, or luciferase. Detection of a test compound which is bound to the immune-related polypeptide can then be accomplished, for example, by direct counting of radioemmission, by scintillation counting, or by determining conversion of an appropriate substrate to a detectable product.

Alternatively, binding of a test compound to an immune-related polypeptide can be determined without labeling either of the interactants. For example, a microphysiometer can be used to detect binding of a test compound with an immune-related polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and an immune-related polypeptide (McConnell et al., Science 257, 1906-1912, 1992).

Determining the ability of a test compound to bind to an immune-related polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an immune-related polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., Biotechniques 14, 920-924, 1993; Iwabuchi et al., Oncogene 8, 1693-1696, 1993; and Brent W094/10300), to identify other proteins which bind to or interact with the immune-related polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding an immune-related polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the immune-related polypeptide.

It may be desirable to immobilize either the immune-related polypeptide (or polynucleotide) or the test compound to facilitate separation of bound from unbound forms of one or both of the interactants, as well as to accommodate automation of the assay. Thus, either the immune-related polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the immune-related polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to an immune-related polypeptide (or polynucleotide) can be accomplished in any vessel suitable for containing the reactants. Examples of such vessels include microtiter plates, test tubes, and microcentrifuge tubes.

In one embodiment, the immune-related polypeptide is a fusion protein comprising a domain that allows the immune-related polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed immune-related polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either an immune-related polypeptide (or polynucleotide) or a test compound can be immobilized utilizing conjugation of biotin and streptavidin. Biotinylated immune-related polypeptides (or polynucleotides) or test compounds can be prepared from biotin-NHS (N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to an immune-related polypeptide, polynucleotide, or a test compound, but which do not interfere with a desired binding site, such as the active site of the immune-related polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the immune-related polypeptide or test compound, enzyme-linked assays which rely on detecting an activity of the immune-related polypeptide, and SDS gel electrophoresis under non-reducing conditions.

Screening for test compounds which bind to an immune-related polypeptide or polynucleotide also can be carried out in an intact cell. Any cell which comprises an immune-related polypeptide or polynucleotide can be used in a cell-based assay system. An immune-related polynucleotide can be naturally occurring in the cell or can be introduced using techniques such as those described above. Binding of the test compound to an immune-related polypeptide or polynucleotide is determined as described above.

Functional Assays

Test compounds can be tested for the ability to increase or decrease a biological effect of an immune-related polypeptide. Such biological effects can be determined using the functional assays described in the specific examples, below. Functional assays can be carried out after contacting either a purified immune-related polypeptide, a cell membrane preparation, or an intact cell with a test compound. A test compound which decreases a functional activity of an immune-related by at least about 10, preferably about 50, more preferably about 75, 90, or 100% is identified as a potential agent for decreasing immune-related activity. A test compound which increases immune-related activity by at least about 10, preferably about 50, more preferably about 75, 90, or 100% is identified as a potential agent for increasing immune-related activity.

One such screening procedure involves the use of B-lymphoma cells which are transfected to express an immune-related polypeptide. For example, such an assay may be employed for screening for a compound which inhibits activation of the polypeptide by exposing the transfected B-lymphoma cells which comprise the polypeptide with both endogenously interacting proteins or substrates to a test compound to be screened. Inhibition of the activity of the polypeptide indicates that a test compound is a potential antagonist for the polypeptide, i.e., inhibits the function of the protein. The screen may be employed for identifying a test compound which activates the protein by exposing such cells to compounds to be screened and determining whether each test compound activates the protein.

Other screening techniques include the use of cells which express a human immune-related polypeptide (for example, transfected T cells) in a system which measures amounts of secreted proteins generated by polypeptide activation. For example, test compounds may be added to cells that express a human immune-related polypeptide and the expression of a reporter gene with specific promoter sequences can be measured to determine whether the test compound activates or inhibits the protein.

Details of functional assays, such as those described above, are provided in the specific examples below.

Gene Expression

In another embodiment, test compounds which increase or decrease immune-related gene expression are identified. An immune-related polynucleotide is contacted with a test compound, and the expression of an RNA or polypeptide product of the immune-related polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of immune-related mRNA or polypeptide expression in the cells can be determined by methods well known in the art for detecting mRNA or polypeptide. Either qualitative or quantitative methods can be used. The presence of polypeptide products of an immune-related polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into an immune-related polypeptide.

Such screening can be carried out either in a cell-free assay system or in an intact cell. Any cell which expresses an immune-related polynucleotide can be used in a cell-based assay system. The immune-related polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, an immune-related polypeptide, immune-related polynucleotide, antibodies which specifically bind to an immune-related polypeptide, or mimetics, enhancers and inhibitors, or inhibitors of an immune-related polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Immune-related polypeptide of the present invention is responsible for many biological functions, including many pathologies. Accordingly, it is desirable to find compounds and drugs which stimulate Immune-related polypeptide on the one hand and which can inhibit the function of an immune-related polypeptide on the other hand. Compounds which can modulate the function or expression of immune-related polypeptide are useful in treating various allergic diseases, autoimmune diseases, inflammatory diseases, and infectious diseases including asthma, allergic rhinitis, atopic dermatitis, hives, conjunctivitis, vernal catarrh, chronic arthrorheumatism, systemic lupus erythematosus, myasthenia gravis, psoriasis, diabrotic colitis, systemic inflammatory response syndrome (SIRS), lymphofollicular thymitis, sepsis, polymyositis, dermatomyositis, polyaritis nodoa, mixed connective tissue disease (MCTD), Sjoegren's syndrome, gout, and the like.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or an immune-related polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects immune-related polypeptide activity can be administered to a human cell, either in vitro or in vivo, to reduce immune-related activity. The reagent preferably binds to an expression product of a human Immune-related polypeptide gene. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a tumor cell, such as a tumor cell ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using protein-mediated targeted delivery. Protein-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 54246 (1994); Zenke et al., Proc. Natl. Acad. Sci. USA 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases immune-related activity relative to the immune-related activity that occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of an immune-related gene or the activity of an immune-related polypeptide by at least about 10, preferably about 50, more preferably about 75, 90, or 100% relative to the absence of the reagent. The effectiveness of the mechanism chosen to decrease the level of expression of an immune-related gene or the activity of an immune-related polypeptide can be assessed using methods well known in the art, such as hybridization of nucleotide probes to immune-related-specific mRNA, quantitative RT-PCR, immunologic detection of an immune-related polypeptide, or measurement of immune-related activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Immune-related polypeptides also can be used in diagnostic assays for detecting diseases and abnormalities or susceptibility to diseases and abnormalities related to the presence of mutations in the nucleic acid sequences which encode a immune-related polypeptide. Such diseases, by way of example, are related to various allergic diseases, autoimmune diseases, inflammatory deseases, and infectious deseases including asthma, allergic rhinitis, atopic dermatitis, hives, conjunctivitis, vernal catarrh, chronic arthrorheumatism, systemic lupus erythematosus, myasthenia gravis, psoriasis, diabrotic colitis, systemic inflammatory response syndrome (SIRS), llymphofollicular thymitis, sepsis, polymyositis, dermatomyositis, polyaritis nodoa, mixed connective tissue disease (MCTD), Sjoegren's syndrome, gout, and the like.

Differences can be determined between the cDNA or genomic sequence encoding a immune-related polypeptide in individuals afflicted with a disease and in normal individuals. If a mutation is observed in some or all of the afflicted individuals but not in normal individuals, then the mutation is likely to be the causative agent of the disease.

Sequence differences between a reference gene and the direct DNA sequencing method can reveal a gene having mutations. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high-resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a Immune-related polypeptide also can be detected in various tissues. Assays used to detect levels of the protein polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Since IL-4 plays a major role in many immune disorders, such as allergy, atopy, and asthma, the Stat6^{-/-} knockout mouse was used to identify genes whose transcription can be activated by IL-4 signaling. To do so, a subtractive hybridization procedure was used to isolate gene transcripts that were expressed differently in IL-4-stimulated T cells and B cells from wildtype mice, whose IL-4 signaling pathway is intact, compared to those from Stat6^{-/-} mice, who lack the Stat6 component of the pathway.

Subtractive Hybridization Study of T and B Cells from Spleens of Stat6^{-/-} Knockout Mice and C57BL/6 Wildtype Mice.

Mice

Stat6^{-/-} mice (C57BL/6 background), 8 weeks old, female, obtained from Professor Shizuo Akira, Osaka University

C57BL/6 wildtype mice, 7.5 weeks old, female, C57BL/6NCrj from Charles River Japan

Tissues

Spleens removed from 4 untreated Stat6^{-/-} and 4 untreated wildtype mice after cervical dislocation. Spleens stored in DMEM-5 until lymphocyte preparation.

Leukocyte Preparation

Spleens tissue was manually disrupted and cells recovered by filtering through a nylon screen and pelleting at 1000 rpm for 10 min. Red blood cells were lysed by resuspending the cells in ACK lysis buffer and incubating for 5 min at room temperature, after which the remaining white blood cells were washed twice with DMEM-5 and counted.

B Cell Isolation

B cells were enriched using the Cellect-Plus mouse B cell kit (Cytovax Biotechnologies, Inc., Edmonton, Alberta, Canada) which removes T cells and macrophages by negative selection. After enrichment, 3.4×10⁷ B cells from Stat6^{-/-} mice and 3.0×10⁷ B cells from wildtype mice were recovered. 3.0×10⁷ B cells from each sample were then incubated at 37° C. for 72 hours in 10 ml DMEM-5 containing 125 ng/ml recombinant mouse interleukin-4 (R&D Systems, Minneapolis, Minn., USA).

T Cell Isolation

T cells were enriched using the Cellect-Plus mouse T cell kit (Cytovax Biotechnologies, Inc., Edmonton, Alberta, Canada) which removes goat-anti-mouse IgG (H+L)-reactive cells. After enrichment, 1.0×10⁸ T cells from Stat6^{-/-} mice and 1.0×10⁸ T cells from wildtype mice were recovered. 1.0×10⁸ T cells from each sample were then incubated at 37 ° C. for 72 hours in 10 ml DMEM-5 containing 125 ng/ml recombinant mouse interleukin-4 (R&D Systems, Minneapolis, Minn., USA) and 2 ng/ml phorbol 12-myristate 13-acetate (Sigma, St. Louis, Mo., USA).

Poly-A mRNA Isolation from Cultured Lymphocytes

T and B cells were collected from culture by gentle pipetting to minimize recovery of any residual adherant cells remaining after the enrichment procedures. Cell yields were as follows: Stat6^{-/-} T cells, 0.5 ×10⁷ cells; Stat6^{-/-} B cells, 0.6 ×10⁷ cells; wildtype T cells, 1.2 ×10⁷ cells; wildtype B cells, 0.7 ×10⁷ cells. Poly-A mRNA was then isolated using the Poly(A) Pure mRNA Isolation Kit (Ambion, Inc., Austin, Tex., USA).

Subtractive Hybridization

Subtractive hybridization using the PCR-Select cDNA Subtraction Kit (Clontech Laboratories, Inc., Palo Alto, Calif., USA) was carried out with pairs of the isolated mRNA populations to remove those gene transcripts expressed in both mRNA populations and thereby enrich for transcripts expressed in one population but not the other. Briefly, cDNA was synthesized from each of the four isolated mRNA populations and divided into “tester” and “driver” aliquots for each population. The cDNAs were then digested with the restriction enzyme RsaI and adaptor molecules were ligated onto the tester aliquots. Tester cDNAs from one mRNA population were then mixed with an excess of driver cDNAs from another population, denatured, and allowed to hybridize. Since cDNA species in the tester population hybridizing with counterparts in the driver population will produce a hybrid with an adaptor on only the tester-derived strand, while those having no driver counterparts will rehybridize with their own tester complements to produce a double-stranded cDNA with adaptors on both strands, a polymerase chain reaction (PCR) could then be performed after filling in the ends and using primers specific for the adaptor sequence which would preferentially amplify those species with adaptors at both ends. This reaction exponentially amplify tester-tester cDNA hybrids and only linearly amplify the tester strand of tester-driver hybrids. After thus enriching for tester-specific species, PCR products were cloned into the pCRII-TOPO cloning vector and sequenced on an ABI Prism 377 DNA sequencer (Applied Biosystems, Foster City, Calif., USA).

Below is a list of the pairs of mRNA populations used in subtractive hybridization experiments:

| Subtraction | Tester mRNA | Driver mRNA |
|---|---|---|
| 1. WTT | wildtype T cells | Stat6^{-/-}T cells |
| 2. WTB | wildtype B cells | Stat6^{-/-}B cells |
| 3. KOT | Stat6^{-/-}T cells | wildtype T cells |
| 4. KOB | Stat6^{-/-}B cells | wildtype B cells |

Analysis of PCR Products

Sequences of PCR products obtained from the individual subtractive hybridization experiments were compared against sequences in the Genbank sequence databases to identify matches with previously annotated sequences or or with unannotated ESTs. Sequences matching against non-coding RNAs, such as ribosomal RNA, were excluded from further analysis.

Sequences found are listed in the table below.

| | Sub- | | | | | | |
|---|---|---|---|---|---|---|---|
| | trac- | Length | Nucleic Acid | Amino Acid | | Gene/Protein | |
| Seq. | tion | (bp) | Seq ID | Seq ID | Matching EST | Match | Annotation |
| 1 | WTT | 1037 | SEQ ID NO: 1 | | | | Novel |
| 2 | WTT | 603 | SEQ ID NO: 2 | | gb\|BE631434 | | Novel |
| 3 | WTB | 243 | SEQ ID NO: 3 | | gb\|AI253400 | | Novel |
| 4 | WTB | 764 | SEQ ID NO: 4 | SEQ ID NO: 5 | gb\|BF161955 | gb\|AF230381_1 | Moderately |
| | | | | | | | similar to p36 |
| | | | | | | | TRAP/SMCC/ |
| | | | | | | | PC2 subunit |
| | | | | | | | [Homo sapiens] |
| 5 | WTB | 228 | SEQ ED NO: 6 | | gb\|BE654711 | | Novel |
| 6 | WTB | 218 | SEQ BD NO: 7 | | dbj\|BB516964 | | Novel |
| 7 | WTB | 230 | SEQ ID NO: 8 | | dbj\|AV052387 | | Novel |
| 8 | WTB | 157 | SEQ ID NO: 9 | | gb\|AA933382 | emb\|HSA012375 | Highly similar |
| | | | | | | | to Homo |
| | | | | | | | sapiens mRNA |
| | | | | | | | for SUI1 |
| | | | | | | | protein |
| | | | | | | | translation |
| | | | | | | | initiation factor |
| 9 | WTB | 374 | SEQ ID NO: 10 | | gb\|AW018385 | | Novel |
| 10 | WTB | 318 | SEQ ID NO: 11 | | gb\|AI686597 | | Novel |
| 11 | WTB | 211 | SEQ ID NO: 12 | | gb\|AI448735 | | Novel |
| 12 | WTB | 710 | SEQ ID NO: 13 | | gb\|AI068585 | | Novel |
| 13 | WTB | 282 | SEQ ID NO: 14 | | gb\|AW682245 | | Novel |
| 14 | WTB | 232 | SEQ ID NO: 15 | | gb\|BE226426 | | Novel |
| 15 | WTB | 654 | SEQ ID NO: 16 | | | | Novel |
| 16 | WTB | 466 | SEQ ID NO: 17 | | | | Novel |
| 17 | WTB | 844 | SEQ ID NO: 18 | SEQ ID NO: 19 | gb\|AI642629 | dbj\|BAA23695.2\| | Highly similar |
| | | | | | | | to KIAA0399 |
| | | | | | | | protein [Homo |
| | | | | | | | sapiens] |
| 18 | WTB | 338 | SEQ ID NO: 20 | SEQ ID NO: 21 | gb\|AA739439 | sp\|ALC_MOUSE | Moderately |
| | | | | | | | similar to IG |
| | | | | | | | ALPHA |
| | | | | | | | CHAIN C |
| | | | | | | | REGION [Mus |
| | | | | | | | musculus] |
| 19 | WTB | 279 | SEQ ID NO: 22 | SEQ ID NO: 23 | gb\|AI550943 | gb\|AF302077_1 | Moderately |
| | | | | | | | similar to |
| | | | | | | | neprilysin-like |
| | | | | | | | peptidase |
| | | | | | | | gamma [Mus |
| | | | | | | | musculus] |
| 20 | WTB | 441 | SEQ ID NO: 24 | | gb\|AA537562 | | Novel |
| 21 | WTB | 290 | SEQ ID NO: 25 | SEQ ID NO: 26 | gb\|BE240836 | sp\|Q59296\| | Highly similar |
| | | | | | | CATA_CAMJE | to Catalase |
| | | | | | | | [Campylobacter |
| | | | | | | | jejuni] |
| 22 | WTB | 462 | SEQ ID NO: 27 | SEQ ID NO: 28 | dbj\|AV312946 | gb\|AF249296 | Highly similar |
| | | | | | | | to Mus |
| | | | | | | | musculus |
| | | | | | | | dihydropyrimi- |
| | | | | | | | dinase mRNA, |
| | | | | | | | complete cds |
| 23 | WTB | 452 | SEQ ID NO: 29 | SEQ ID NO: 30 | gb\|AW210281 | dbj\|BAB15170.1\| | Moderately |
| | | | | | | | similar to |
| | | | | | | | unnamed |
| | | | | | | | protein product |
| | | | | | | | [Homo sapiens] |
| 24 | WTB | 469 | SEQ ID NO: 31 | | | | Novel |
| 25 | WTB | 384 | SEQ ID NO: 32 | SEQ ID NO: 33 | gb\|BF140918 | gb\|AAA64268.1\| | Moderately |
| | | | | | | | similar to |
| | | | | | | | neural- |
| | | | | | | | restrictive |
| | | | | | | | silencer factor |
| | | | | | | | [Mus |
| | | | | | | | musculus] |
| 26 | WTB | 769 | SEQ ID NO: 34 | | gb\|AI314055 | | Novel |
| 27 | WTB | 442 | SEQ ID NO: 35 | | gb\|AI267507 | | Novel |
| 28 | WTB | 703 | SEQ ID NO: 36 | | gb\|AW540884 | | Novel |
| 29 | WTB | 1091 | SEQ ID NO: 37 | SEQ ID NO: 38 | dbj\|AV318321 | sp\|P29374\| | Moderately |
| | | | | | | RBB1_HUMAN | similar to |
| | | | | | | | RETINOBLAS- |
| | | | | | | | TOMA |
| | | | | | | | BINDING |
| | | | | | | | PROTEIN 1 |
| | | | | | | | (RBBP-1) |
| | | | | | | | [Homo sapiens] |
| 30 | WTB | 750 | SEQ ID NO: 39 | | gb\|BF321136 | | Novel |
| 31 | WTB | 324 | SEQ ID NO: 40 | | gb\|AF017689 | dbj\|AB019573 | Highly similar |
| | | | | | | | to Homo |
| | | | | | | | sapiens mRNA |
| | | | | | | | expressed only |
| | | | | | | | in placental |
| | | | | | | | villi, clone |
| | | | | | | | SMAP31 |
| 32 | WTB | 680 | SEQ ID NO: 41 | | gb\|AW231940 | | Novel |
| 33 | WTB | 292 | SEQ ID NO: 42 | | gb\|AW821223 | | Novel |
| 34 | KOT- | 524 | SEQ ID NO: 43 | SEQ ID NO: 44 | gb\|BE289541 | sp\|Q13243\|SFR5_{—} | Highly similar |
| | B | | | | | HUMAN | to SPLICING |
| | | | | | | | FACTOR, |
| | | | | | | | ARGININE/SER- |
| | | | | | | | INE-RICH 5 |
| | | | | | | | (PRE-MRNA |
| | | | | | | | SPLICING |
| | | | | | | | FACTOR |
| | | | | | | | SRP40) |
| | | | | | | | (DELAYED- |
| | | | | | | | EARLY |
| | | | | | | | PROTEIN |
| | | | | | | | HRS) [H |
| | | | | | | | sapiens] |
| 35 | KOT | 926 | SEQ ID NO: 45 | | | | Novel |
| 36 | KOT | 564 | SEQ ID NO: 46 | | gb\|AW503483 | | Novel |
| 37 | KOT | 199 | SEQ ID NO: 47 | | gb\|AI267534 | | Novel |
| 38 | KOT | 868 | SEQ ID NO: 48 | | | | Novel |
| 39 | KOT | 763 | SEQ ID NO: 49 | | | | Novel |
| 40 | KOT | 394 | SEQ ID NO: 50 | SEQ ID NO: 51 | | emb\|X70057.1\| | Highly similar |
| | | | | | | MMSRPRTSA | to M. musculus |
| | | | | | | | serine |
| | | | | | | | proteinase gene |
| 41 | KOT | 248 | SEQ ID NO: 52 | | gb\|AW681658 | | Novel |
| 42 | KOT | 207 | SEQ ID NO: 53 | | gb\|AA590726 | | Novel |
| 43 | KOT | 113 | SEQ ID NO: 54 | SEQ ID NO: 55 | | emb\|X70057.1\| | Highly similar |
| | | | | | | MMSRPRTSA | to M. musculus |
| | | | | | | | serine |
| | | | | | | | proteinase gene |
| 44 | KOT | 576 | SEQ ID NO: 56 | | dbj\|BB235049 | | Novel |
| 45 | KOT | 686 | SEQ ID NO: 57 | | dbj\|AV165785 | | Novel |
| 46 | KOT | 797 | SEQ ID NO: 58 | | gb\|AW681327 | | Novel |
| 47 | KOT | 611 | SEQ ID NO: 59 | | gb\|AW121041 | | Novel |
| 48 | KOT | 743 | SEQ ID NO: 60 | | | | Novel |
| 49 | KOT | 934 | SEQ ID NO: 61 | | gb\|AI620970 | | Novel |
| 50 | KOT | 776 | SEQ ID NO: 62 | | | | Novel |
| 51 | KOT | 286 | SEQ ID NO: 63 | | dbj\|AV247290 | gb\|AC005403 | Moderately |
| | | | | | | | similar to Mus |
| | | | | | | | musculus clone |
| | | | | | | | UWGC: ma53a0 |
| | | | | | | | 68 from 14D1- |
| | | | | | | | D2 (T-Cell |
| | | | | | | | Receptor Alpha |
| | | | | | | | Locus) |
| 52 | KOT | 363 | SEQ ID NO: 64 | | dbj\|BB224578 | | Novel |
| 53 | KOT | 941 | SEQ ID NO: 65 | | | | Novel |
| 54 | KOT | 95 | SEQ ID NO: 66 | | | gb\|AF151726 | Highly similar |
| | | | | | | | to Dianthus |
| | | | | | | | caryophyllus |
| | | | | | | | putative MtN3- |
| | | | | | | | like protein |
| | | | | | | | mRNA, |
| | | | | | | | complete cds |
| 55 | KOT | 533 | SEQ ID NO: 67 | | gb\|AW288466 | | Novel |
| 56 | KOT | 304 | SEQ ID NO: 68 | | dbj\|AV218547 | | Novel |
| 57 | KOT | 658 | SEQ ID NO: 69 | | | | Novel |
| 58 | KOT | 593 | SEQ ID NO: 70 | SEQ ID NO: 71 | gb\|AA065486 | sp\|GS28_CRIGR | Moderately |
| | | | | | | | similar to 28 |
| | | | | | | | KDA GOLGI |
| | | | | | | | SNARE |
| | | | | | | | PROTEIN |
| | | | | | | | (GOLGI SNAP |
| | | | | | | | RECEPTOR |
| | | | | | | | COMPLEX |
| | | | | | | | MEMBER 1) |
| | | | | | | | (28 KDA CIS- |
| | | | | | | | GOLGI |
| | | | | | | | SNARE P28) |
| | | | | | | | (GOS-28) |
| | | | | | | | [Cricetulus |
| | | | | | | | griseus] |
| 59 | KOT | 919 | SEQ ID NO: 72 | | gb\|AI619748 | | Novel |
| 60 | KOT | 886 | SEQ ID NO: 73 | | gb\|AI619502 | | Novel |
| 61 | KOT | 848 | SEQ ID NO: 74 | | | | Novel |
| 62 | KOT | 386 | SEQ ID NO: 75 | | | | Novel |
| 63 | KOT | 707 | SEQ ID NO: 76 | SEQ ID NO: 77 | | gb\|AF006466 | Moderately |
| | | | | | | | similar to Mus |
| | | | | | | | musculus |
| | | | | | | | lymphocyte |
| | | | | | | | specific formin |
| | | | | | | | related protein |
| | | | | | | | (Fr1) mRNA, |
| | | | | | | | complete cds |
| 64 | KOT | 287 | SEQ ID NO: 78 | SEQ ID NO: 79 | gb\|AW541155 | emb\|AJ000008.1\| | Highly similar |
| | | | | | | HSC2PI3KI | to Homo |
| | | | | | | | sapiens mRNA |
| | | | | | | | for C2 domain |
| | | | | | | | containing PI3- |
| | | | | | | | kinase |
| 65 | KOT | 922 | SEQ ID NO: 80 | SEQ ID NO: 81 | gb\|AA592138 | gb\|AF149204.1\| | Highly similar |
| | | | | | | AF149204 | to Mus |
| | | | | | | | musculus |
| | | | | | | | Su(var)3-9 |
| | | | | | | | homolog |
| | | | | | | | Suv39h2 |
| | | | | | | | (Suv39h2) gene |
| 66 | KOT | 360 | SEQ ID NO: 82 | SEQ ID NO: 83 | gb\|AA154868 | gb\|AF118128 | Highly similar |
| | | | | | | | to Mus |
| | | | | | | | musculus |
| | | | | | | | nuclear RNA |
| | | | | | | | helicase Bat1 |
| | | | | | | | mRNA, |
| | | | | | | | complete cds |
| 67 | KOB | 332 | SEQ ID NO: 84 | | gb\|AW681928 | | Novel |
| 68 | KOB | 255 | SEQ ID NO: 85 | SEQ ID NO: 86 | gb\|AA145022 | sp\|PI4K_HUMAN | Highly similar |
| | | | | | | | to |
| | | | | | | | PHOSPHATID- |
| | | | | | | | YLINOSITOL |
| | | | | | | | 4-KINASE |
| | | | | | | | ALPHA (PI4- |
| | | | | | | | KINASE) |
| | | | | | | | (PTDINS-4- |
| | | | | | | | KINASE) |
| | | | | | | | (PI4K-ALPHA) |
| | | | | | | | [Homo |
| | | | | | | | Sapiens] |
| 69 | KOB | 610 | SEQ ID NO: 87 | | gb\|AA185612 | gb\|AF244347 | Moderately |
| | | | | | | | similar to Mus |
| | | | | | | | musculus ADP- |
| | | | | | | | ribosylarginine |
| | | | | | | | hydrolase |
| | | | | | | | mRNA, |
| | | | | | | | complete cds |
| 70 | KOB | 401 | SEQ ID NO: 88 | | gb\|AI253322 | | Novel |
| 71 | KOB | 639 | SEQ ID NO: 89 | SEQ ID NO: 90 | gb\|AI662232 | pir\|S12207 | Moderately |
| | | | | | | | similar to |
| | | | | | | | hypothetical |
| | | | | | | | protein (B2 |
| | | | | | | | element) - |
| | | | | | | | mouse |
| 72 | KOB | 257 | SEQ ID NO: 91 | | gb\|BF459040 | gb\|MMU29539 | Highly similar |
| | | | | | | | to Mus |
| | | | | | | | musculus |
| | | | | | | | retinoic acid- |
| | | | | | | | inducible E3 |
| | | | | | | | protein mRNA, |
| | | | | | | | complete cds |
| 73 | KOB | 499 | SEQ ID NO: 92 | SEQ ID NO: 93 | dbj\|AU079390 | sp\|PAPS_BAXSU | Weakly similar |
| | | | | | | | to POLY(A) |
| | | | | | | | POLYMERASE |
| | | | | | | | (PAP) |
| 74 | KOB | 711 | SEQ ID NO: 94 | SEQ ID NO: 95 | | gb\|MMU67328 | Highly similar |
| | | | | | | | to Mus |
| | | | | | | | musculus NIPI- |
| | | | | | | | like protein |
| | | | | | | | (NIPIL(A3)) |
| | | | | | | | mRNA, |
| | | | | | | | complete cds |
| 75 | KOB | 276 | SEQ ID NO: 96 | SEQ ID NO: 97 | dbj\|AV001836 | gb\|RNU38801 | Moderately |
| | | | | | | | similar to |
| | | | | | | | Rattus |
| | | | | | | | norvegicus high |
| | | | | | | | molecular |
| | | | | | | | weight DNA |
| | | | | | | | polymerase beta |
| | | | | | | | (mpolb) |
| | | | | | | | mRNA, |
| | | | | | | | complete cds |
| 76 | KOB | 419 | SEQ ID NO: 98 | SEQ ID NO: 99 | gb\|AI596571 | dbj\|BAA76376.1\| | Moderately |
| | | | | | | | similar to Trif |
| | | | | | | | [Mus |
| | | | | | | | musculus] |
| 77 | KOB | 205 | SEQ ID | | gb\|AI963068 | | Novel |
| | | | NO: 100 | | | | |
| 78 | KOB | 752 | SEQ ID | SEQ ID | gb\|AW325099 | gb\|AAB81227.1\| | Highly similar |
| | | | NO: 101 | NO: 102 | | | to alpha- |
| | | | | | | | hemolysin |
| | | | | | | | [Aeromonas |
| | | | | | | | hydrophila] |
| 79 | KOB | 180 | SEQ ID | SEQ ID | gb\|AA023730 | sp\|HA11_MOUSE | Moderately |
| | | | NO: 103 | NO: 104 | | | similar to H-2 |
| | | | | | | | CLASS I |
| | | | | | | | HISTOCOMPAT- |
| | | | | | | | IBILITY |
| | | | | | | | ANTIGEN, D- |
| | | | | | | | B ALPHA |
| | | | | | | | CHAIN |
| | | | | | | | PRECURSOR |
| | | | | | | | (H-2D(B)) |
| | | | | | | | [Mus |
| | | | | | | | Musculus] |
| 80 | KOB | 398 | SEQ ID | | dbj\|BB564868 | | Novel |
| | | | NO: 105 | | | | |
| 81 | KOB | 376 | SEQ ID | SEQ ID | gb\|AI235753 | dbj\|D78130 | Highly similar |
| | | | NO: 106 | NO: 107 | | | to Homo |
| | | | | | | | sapiens mRNA |
| | | | | | | | for squalene |
| | | | | | | | epoxidase |
| 82 | KOB | 261 | SEQ ID | | gb\|BE896297 | | Novel |
| | | | NO: 108 | | | | |
| 83 | KOB | 478 | SEQ ED | | gb\|BE137653 | | Novel |
| | | | NO: 109 | | | | |
| 84 | KOB | 871 | SEQ ID | | dbj\|AV718662 | | Novel |
| | | | NO: 110 | | | | |
| 85 | KOB | 175 | SEQ ID | | gb\|AI249085 | | Novel |
| | | | NO: 111 | | | | |

In the two subtractions using wildtype mRNA as the tester and Stat6^{-/-} mRNA as the driver, it is expected that the majority of enriched transcripts will be from genes activated by stimulation of the wildtype T and B cells by IL-4. These genes will therefore be important targets for regulation in the treatment of IL-4 mediated disorders. On the other hand, in the two subtractions using Stat6^{-/-} mRNA as the tester and wildtype mRNA as the driver, it is expected that the majority of enriched transcripts will be from genes either normally activated in Stat6^{-/-} cells as a compensatory mechanism for the lack of Stat6, or from normally active genes that are turned off in wildtype T and B cells upon stimulation by IL-4. These genes may therefore be important targets for enhancement in the treatment of IL-4 mediated disorders.

### EXAMPLE 2

Preparation of Full Length cDNA Clones

The immune-related polypeptide sequences presented in the Example 1 can be used to design oligonucleotide primers for the extension of the cDNAs to fall length. A pair of primers consists of a primer synthesized to initiate extension in the antisense direction and a primer synthesized to extend sequence in the sense direction. The primers allow the sequence to be extended outward generating amplicons containing new nucleotide sequence for the immune-related gene. The primers are annealed to the target sequence at temperatures about 68 to 72°C. The spleen cDNA library are used as a template. Preferably, cDNA prepared from IL-4-stimulated T cells and B cells from wildtype mice, whose IL-4 signaling pathway is intact, and IL-4 stimulated T cells and B cells from Stat6^{-/-} mice are used as a template.

### EXAMPLE 3

Tissue Expression of Immune-related Polypeptide mRNA

Quantitative reverse transcription-polymerase chain reaction (RT-PCR) analysis of RNA from different human tissues is performed to investigate the tissue expression of immune-related polypeptide mRNA. 100 .mu.g of total RNA from various tissues (Human Total RNA Panel I-V, Clontech Laboratories, Palo Alto, Calif., USA) is used as a template to synthsize first-strand cDNA using the SUPERSCRIPT™ First-Strand Syntheswas System for RT-PCR (Life Technologies, Rockville , Md., USA). 10 ng of the first-strand cDNA is then used as template in a polymerase chain reaction to test for the presence of the immune-related polypeptide mRNA transcript. The polymerase chain reaction is performed in a LightCycler (Roche Molecular Biochemicals, Indianapolis, Ind., USA), in the presence of the DNA-binding fluorescent dye SYBR Green I which binds to the minor groove of the DNA double helix, produced only when double-stranded DNA is successfully synthesized in the reaction, and upon binding, emits light that can be quantitatively measured by the LightCycler machine. The polymerase chain reaction is carried out using oligonucleotide primers designed to span the junction of spliced exons flanking deleted regions and measurements of the intensity of emitted light are taken following each cycle of the reaction when the reaction reach a temperature of 86 degrees C. Intensities of emitted light are converted into copy numbers of the gene transcript per nanogram of template cDNA by comparison with simultaneously reacted standards of known concentration.

To correct for differences in mRNA transcription levels per cell in the various tissue types, a normalization procedure is performed using calculated expression levels in the various tissues of five different housekeeping genes: glyceraldehyde-3-phosphatase (G3PHD), hypoxanthine guanine phophoribosyl transferase (HPRT), beta-actin, porphobilinogen deaminase (PBGD), and beta-2-microglobulin. Except for the use of a slightly different set of housekeeping genes, the normalization procedures is essentially the same as that described in the RNA Master Blot User Manual, Apendix C (Clontech Laboratories, Palo Alto, Calif., USA).

### EXAMPLE 4

Functional Characterization

The function of each of the immune-related polypeptides is assessed by their ability of specifically interacting with appropriate proteins. Each of the immune-related cDNA inserts obtained in Example 1 is subcloned into a mammalian expression vector which fuses the coding region to an epitope tag from a influenza hemagglutinin (HA) peptide, vector pCEP4-HA (Herrscher, R. F. et al. (1995) Genes Dev. 9:3067-3082), to create the expression vector.

The vector is then transfected into appropriate cells. The cells are cultured and the cultured cells are recovered to extract the immune-related polypeptides. The polypeptides are then interacted with appropriate proteins.

### EXAMPLE 5

Functional Activity of Immune-related Polypeptides in Response to the IL-4 Stimulation

To test for a functional activity of immune-related polypeptides in response to the IL-4 stimulation, each of the immune-related polypeptides is expressed at high levels in cells expressing low levels of endogenous immune-related polypeptides, and each of the cells is stimulated by IL-4. The activity of each of the immune-related polypeptides is measured.

### EXAMPLE 6

Identification of a Test Compound which Binds to each of Immune-related Polypeptides

Each of the purified immune-related polypeptide comprising a glutathione-S-transferase protein and absorbed onto glutathione-derivatized wells of 96-well microtiter plates are contacted with test compounds from a small molecule library at pH 7.0 in a physiological buffer solution. Immune-related polypeptides comprise any of the amino acid sequence shown in SEQ ID NO:5, 19, 21, 23, 26, 28, 30, 33, 38, 44, 51, 55, 71, 77, 79, 81, 83, 86, 90, 93, 95, 97, 99, 102, 104, and 107. The test compounds comprise a fluorescent tag. The samples are incubated for 5 minutes to one hour. Control samples are incubated in the absence of a test compound.

The buffer solution containing the test compounds is washed from the wells. Binding of a test compound to an immune-related polypeptide is detected by fluorescence measurements of the contents of the wells. A test compound which increases the fluorescence in a well by at least 15% relative to fluorescence of a well in which a test compound is not incubated is identified as a compound which binds to an immune-related polypeptide.

### EXAMPLE 7

Identification of a Test Compound which Modulates (Increases or Decreases) Immune-related Gene Expression

A test compound is administered to a culture of human lymph node cells and incubated at 37° C. for 10 to 45 minutes. A culture of the same type of cells incubated for the same time without the test compound provides a negative control.

RNA is isolated from the two cultures as described in Chirgwin et al., Biochem. 18, 5294-99, 1979). Northern blots are prepared using 20 to 30 μg total RNA and hybridized with a ³²P-labeled immune-related-specific probe at 65 ° C. in Express-hyb (CLONTECH). The probe comprises at least 11 contiguous nucleotides selected from the complement of SEQ ID NOs: 1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, and 111. A test compound which modulates the immune-related-specific signal relative to the signal obtained in the absence of the test compound is identified as an modulator of immune-related gene expression.

### EXAMPLE 8

Screening for a compound which modulates the interaction between Immune-related protein and NF-AT can be done with the use of yeast two-hybrid system (s).

### EXAMPLE 9

Treatment of immunologically related diseases by modulating the function of a human Immune-related.

A polynucleotide which expresses a human Immune-related protein or a compound, which modulate the function of Immune-related protein is administered to a patient, The severity of the patient's inflammation is lessened.

Equivalents

Those skilled in the art will recognize, or be able to ascertain using no more than routine experimentation, many equivalents to the specific embodiments of the invention described herein. Such equivalents are intended to be encompassed by the following claims.

References

1. Takeda K, Tanaka T, Shi W, Matsumoto M, Minami M, Kashiwamura S, Nakanishi K, Yoshida N, Kishimoto T, Akira S. Essential role of Stat6 in IL-4 signalling. Nature. 1996 Apr. 18;380(6575):627-30.
2. Shimoda K, van Deursen J, Sangster M Y, Sarawar S R, Carson R T, Tripp R A, Chu C, Quelle F W, Nosaka T, Vignali D A, Doherty P C, Grosveld G, Paul W E, Ihle J N. Lack of IL-4-induced Th2 response and IgE class switching in mice with disrupted Stat6 gene. Nature. 1996 Apr. 18;380(6575):630-3.
3. Takeda K, Kamanaka M, Tanaka T, Kishimoto T, Akira S. Impaired IL-13-mediated functions of macrophages in Stat6-deficient mice. J Immunol. 1996 Oct. 15;157(8):3220-2.
4. Takeda K, Kishimoto T. Akira S. Stat6: its role in interleukin 4-mediated biological functions. J Mol Med 1997May; 75(5):317-26.

1. An isolated polynucleotide selected from the group consisting of
a) a polynucleotide encoding an Immune-related protein or a protein exhibiting biological properties of a human Immune-related protein and comprising the amino acid sequence of SEQ ID NOS: 5, 19, 21, 23, 26, 28, 30, 33, 38, 44, 51, 55, 71, 77, 79, 81, 83, 86, 90, 93, 95, 97, 99, 102, 104, or 107;
b) a polynucleotide comprising the sequence of SEQ ID NOS:1-4, 6-18, 20, 22, 24, 25, 27, 29, 31, 32, 34, 35, 36, 37, 39-43, 45-50, 52, 53, 54, 56-70, 72-76, 78, 80, 82, 84, 85, 87, 88, 89, 91, 92, 94, 96, 98, 100, 101, 103, 105, 106, 108, 109, 110, or 111;
c) a polynucleotide which hybridizes under stringent conditions to a polynucleotide specified in (a) and (b) and encodes an Immune-related protein or a protein exhibiting biological properties of a human Immune-related protein;
d) a polynucleotide the nucleic acid sequence of which deviates from the nucleic acid sequences specified in (a) to (c) due to the degeneration of the genetic code and encodes an Immune-related protein or a protein exhibiting biological properties of a human Immune-related protein; and
e) a polynucleotide, which represents a fragment, derivative or allelic variation of a nucleic acid sequence specified in (a) to (d) and encodes an Immune-related protein or a protein exhibiting biological properties of a human Immune-related protein.

2. An expression vector containing any polynucleotide sequence of claim 1.

3. A host cell containing the expression vector of claim 2.

4. A substantially purified protein exhibiting biological properties of human Immune-related protein, which is encoded by a polynucleotide of claim 1.

5. A method for producing an isolated protein exhibiting biological properties of human Immune-related protein, the method comprising the steps of:
a) culturing the host cell of claim 4 under conditions suitable for the expression of the polypeptide; and
b) recovering the polypeptide from the host cell culture.

6. A method for the detection of polynucleotides encoding a Immune-related protein or a protein exhibiting biological properties of a human Immune-related protein in a biological sample comprising the steps of:
a) hybridizing any polynucleotide of claim 1 to nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a protein of claim 4 comprising the steps of:
a) contacting a biological sample with a reagent which specifically interacts with the polynucleotide of claim 1 or the protein of claim 4 and
b) detecting the interaction.

9. A diagnostic kit for conducting the method of one of the claims 6, 7 or 8.

10. A method of screening for agents which regulate the activity of human Immune-related protein, comprising the steps of:
a) contacting a test compound with a polypeptide encoded by any of the polynucleotides of claim 1;
b) detecting binding of the test compound to the polypeptide, wherein a test compound which binds to the polypeptide is identified as a potential therapeutic agent for regulating the activity of human Immune-related protein.

11. A method of screening for agents which regulate the activity of human Immune-related protein, comprising the steps of:
a) contacting a test compound with a polypeptide encoded by any of the polynucleotides of claim 1; and
b) detecting a Immune-related protein activity of the polypeptide, wherein a test compound which increases the Immune-related protein activity is identified as a potential therapeutic agent for increasing the activity of the human Immune-related protein, and wherein a test compound which decreases the Immune-related protein activity of the polypeptide is identified as a potential therapeutic agent for decreasing the activity of the human Immune-related protein.

12. A method of screening for agents which regulate the activity of human Immune-related protein, comprising the steps of:
a) contacting a test compound with any polynucleotide of claim 1 and
b) detecting binding of the test compound to any polynucleotide of claim 1, wherein a test compound which binds to the polynucleotide is identified as a potential therapeutic agent for regulating the activity of human Immune-related protein.

13. A method of modulating the activity of human Immune-related protein, comprising the step of:
contacting a cell with a reagent which specifically binds to any polynucleotide of claim 1 or a protein of claim 4, whereby the activity of human Immune-related protein is reduced.

14. A purified reagent that modulates the activity of a human Immune-related protein polypeptide or polynucleotide, wherein said reagent is identified by the method of any of the claims 10, 11 or 12.

15. A pharmaceutical composition, comprising:
a reagent which modulates the activity of a human Immune-related protein polypeptide or polynucleotide, wherein said reagent is identified by the method of claim 10, 11 or 12; and
a pharmaceutically acceptable carrier.

16. A pharmaceutical composition, comprising:
an expression vector of claim 3, and
a pharmaceutically acceptable carrier.

17. Use of the expression vector of claim 2, or the reagent of claim 14 in the preparation of medicament for modulating the activity of immune-related proteins in a disease.

18. Use of claim 16, wherein the disease an allergic disease, an autoimmune disease, an inflammatory disease, or an infectious disease.
